SEQUENCE LISTING (75 sequences)

SEQ ID NO: 1 - DNA, 22 bp, Artificial Sequence
  Synthetic Construct
tagtgaccagctataatcagag

SEQ ID NO: 2 - DNA, 20 bp, Artificial Sequence
  Synthetic Construct
acgccaaggtctgaaggtcc

SEQ ID NO: 3 - DNA, 19 bp, Artificial Sequence
  Synthetic Construct
ccctgctggccctgctctt

SEQ ID NO: 4 - DNA, 20 bp, Artificial Sequence
  Synthetic Construct
aggtctgaaggtcacctgct

SEQ ID NO: 5 - DNA, 20 bp, Artificial Sequence
  Synthetic Construct
cagaggagaaccccagatca

SEQ ID NO: 6 - DNA, 20 bp, Artificial Sequence
  Synthetic Construct
tcatgacgtttggcaatgtt

SEQ ID NO: 7 - DNA, 20 bp, Artificial Sequence
  Synthetic Construct
gaggcaaggaagatgctgtc

SEQ ID NO: 8 - DNA, 20 bp, Artificial Sequence
  Synthetic Construct
agttcttgcagccagctttg

SEQ ID NO: 9 - DNA, 20 bp, Artificial Sequence
  Synthetic Construct
ggcccaacactcactagctc

SEQ ID NO: 10 - DNA, 20 bp, Artificial Sequence
  Synthetic Construct
ccaggaagtccacctgtgtt

SEQ ID NO: 11 - DNA, 20 bp, Artificial Sequence
  Synthetic Construct
gaagccaccagctaaactgc

SEQ ID NO: 12 - DNA, 20 bp, Artificial Sequence
  Synthetic Construct
cggatgtgagttcttgctca

SEQ ID NO: 13 - DNA, 20 bp, Artificial Sequence
  Synthetic Construct
gcaagatcctgagagccaac

SEQ ID NO: 14 - DNA, 20 bp, Artificial Sequence
  Synthetic Construct
ttgttgtcggatcttgtcca

SEQ ID NO: 15 - DNA, 20 bp, Artificial Sequence
  Synthetic Construct
tcagagacggtgcagaaatg

SEQ ID NO: 16 - DNA, 20 bp, Artificial Sequence
  Synthetic Construct
caaggcggagaaagaaagtg

SEQ ID NO: 17 - DNA, 20 bp, Artificial Sequence
  Synthetic Construct
cattgttgcaccttgtcacc

SEQ ID NO: 18 - DNA, 20 bp, Artificial Sequence
  Synthetic Construct
ttctgctgctttccctcatt

SEQ ID NO: 19 - DNA, 20 bp, Artificial Sequence
  Synthetic Construct
ggaaccatcctggctaacaa

SEQ ID NO: 20 - DNA, 20 bp, Artificial Sequence
  Synthetic Construct
ctcagttggaggcaatgaca

SEQ ID NO: 21 - DNA, 18 bp, Artificial Sequence
  Synthetic Construct
gctacggcacagtgcttg

SEQ ID NO: 22 - DNA, 21 bp, Artificial Sequence
  Synthetic Construct
caggattgcagacagatagtc

SEQ ID NO: 23 - DNA, 20 bp, Artificial Sequence
  Synthetic Construct
cctgtgaactctggtatcag

SEQ ID NO: 24 - DNA, 20 bp, Artificial Sequence
  Synthetic Construct
gctcacaccaaagagtcaac

SEQ ID NO: 25 - DNA, 22 bp, Artificial Sequence
  Synthetic Construct
ggaaaggagctgattgctgtcc

SEQ ID NO: 26 - DNA, 22 bp, Artificial Sequence
  Synthetic Construct
ctttgacaaggctggagaccag

SEQ ID NO: 27 - DNA, 20 bp, Artificial Sequence
  Synthetic Construct
ttaaatgccttggccatctc

SEQ ID NO: 28 - DNA, 20 bp, Artificial Sequence
  Synthetic Construct
ccacggagttctctgctttc

SEQ ID NO: 29 - DNA, 20 bp, Artificial Sequence
  Synthetic Construct
ttggctgaaagggaaagaga

SEQ ID NO: 30 - DNA, 20 bp, Artificial Sequence
  Synthetic Construct
gcttcatgtgctctggttga

SEQ ID NO: 31 - DNA, 20 bp, Artificial Sequence
  Synthetic Construct
ctgtgacggctatgcttcaa

SEQ ID NO: 32 - DNA, 20 bp, Artificial Sequence
  Synthetic Construct
agctgcagatgtcccagagt

SEQ ID NO: 33 - DNA, 20 bp, Artificial Sequence
  Synthetic Construct
gaggaggaagaggaggctgt

SEQ ID NO: 34 - DNA, 20 bp, Artificial Sequence
  Synthetic Construct
tgtcctcccattctctggac

SEQ ID NO: 35 - DNA, 21 bp, Artificial Sequence
  Synthetic Construct
cggtgggacttgtgctgctgg

SEQ ID NO: 36 - DNA, 20 bp, Artificial Sequence
  Synthetic Construct
cgcaatgtactggaagcaga

SEQ ID NO: 37 - DNA, 20 bp, Artificial Sequence
  Synthetic Construct
gcctgtgtatgcaaccattg

SEQ ID NO: 38 - DNA, 20 bp, Artificial Sequence
  Synthetic Construct
catttgtggggtgtggagtc

SEQ ID NO: 39 - DNA, 21 bp, Artificial Sequence
  Synthetic Construct
ggctcctagtgacctgcacca

SEQ ID NO: 40 - DNA, 22 bp, Artificial Sequence
  Synthetic Construct
ccacagccacactgcgcttgcg

SEQ ID NO: 41 - DNA, 21 bp, Artificial Sequence
  Synthetic Construct
tggtcactggggacaagggaa

SEQ ID NO: 42 - DNA, 21 bp, Artificial Sequence
  Synthetic Construct
gcaacaacagcaatagagaac

SEQ ID NO: 43 - DNA, 20 bp, Artificial Sequence
  Synthetic Construct
cacgctaccctacgaaaagc

SEQ ID NO: 44 - DNA, 20 bp, Artificial Sequence
  Synthetic Construct
cctctggggtccacacttta

SEQ ID NO: 45 - DNA, 20 bp, Artificial Sequence
  Synthetic Construct
aaatggcgcaggcaagagaa

SEQ ID NO: 46 - DNA, 20 bp, Artificial Sequence
  Synthetic Construct
atgaggaggaagccacagga

SEQ ID NO: 47 - DNA, 25 bp, Artificial Sequence
  Synthetic Construct
gcttcatccgagtcttctccgttag

SEQ ID NO: 48 - DNA, 22 bp, Artificial Sequence
  Synthetic Construct
ccatctttgcttgggaaatccg

SEQ ID NO: 49 - DNA, 22 bp, Artificial Sequence
  Synthetic Construct
cttggccaagaactacatctgg

SEQ ID NO: 50 - DNA, 21 bp, Artificial Sequence
  Synthetic Construct
ggagtagggatgcaccgggaa

SEQ ID NO: 51 - DNA, 22 bp, Artificial Sequence
  Synthetic Construct
agatatgggagacatgggcgat

SEQ ID NO: 52 - DNA, 21 bp, Artificial Sequence
  Synthetic Construct
acacagcggaaacactcgatg

SEQ ID NO: 53 - DNA, 20 bp, Artificial Sequence
  Synthetic Construct
aaccgtgccacgcgctcaaa

SEQ ID NO: 54 - DNA, 21 bp, Artificial Sequence
  Synthetic Construct
agggcctaaggcctccagtct

SEQ ID NO: 55 - DNA, 20 bp, Artificial Sequence
  Synthetic Construct
atcatcactctgcccaccat

SEQ ID NO: 56 - DNA, 20 bp, Artificial Sequence
  Synthetic Construct
agtcggatgacctcctcctt

SEQ ID NO: 57 - DNA, 20 bp, Artificial Sequence
  Synthetic Construct
ccaccccagtttacaagctc

SEQ ID NO: 58 - DNA, 20 bp, Artificial Sequence
  Synthetic Construct
tgtaggcagtacgggtcctc

SEQ ID NO: 59 - DNA, 24 bp, Artificial Sequence
  Synthetic Construct
ctgcgcatagcggaccacagcttc

SEQ ID NO: 60 - DNA, 26 bp, Artificial Sequence
  Synthetic Construct
cttcacaagaagtctgagaacaccag

SEQ ID NO: 61 - DNA, 20 bp, Artificial Sequence
  Synthetic Construct
aaaaggaaaggcggtcaagt

SEQ ID NO: 62 - DNA, 20 bp, Artificial Sequence
  Synthetic Construct
ctgctcacaggaagtgtcca

SEQ ID NO: 63 - DNA, 20 bp, Artificial Sequence
  Synthetic Construct
atgaagatcctgaccgagcg

SEQ ID NO: 64 - DNA, 20 bp, Artificial Sequence
  Synthetic Construct
tacttgcgctcaggaggagc

SEQ ID NO: 65 - DNA, 32 bp, Artificial Sequence
  Synthetic Construct
gactgctctagacaaccgtgtaaatgccactg

SEQ ID NO: 66 - DNA, 32 bp, Artificial Sequence
  Synthetic Construct
gactgcaagctttgagcatccacctctgtgtt

SEQ ID NO: 67 - DNA, 32 bp, Artificial Sequence
  Synthetic Construct
gacattcccgggacactggagaagggggttct

SEQ ID NO: 68 - DNA, 50 bp, Artificial Sequence
  Synthetic Construct
gactgtctcgaggccggcgcggccgcccatgggcttgctgatggtctctg

SEQ ID NO: 69 - DNA, 20 bp, Artificial Sequence
  Synthetic Construct
tgaccactgtgcttctgagg

SEQ ID NO: 70 - DNA, 20 bp, Artificial Sequence
  Synthetic Construct
ggggaatgatgtggaaaatg

SEQ ID NO: 71 - DNA, 20 bp, Artificial Sequence
  Synthetic Construct
aggtgcttctcgatctgcat

SEQ ID NO: 72 - DNA, 32 bp, Artificial Sequence
  Synthetic Construct
gactgccccgggcaaccgtgtaaatgccactg

SEQ ID NO: 73 - DNA, 46 bp, Artificial Sequence
  Synthetic Construct
gactgccccgggtcagctgcggccgcctgctgccatgactacctga

SEQ ID NO: 74 - DNA, 20 bp, Artificial Sequence
  Synthetic Construct
cagccaccattacaatgcac

SEQ ID NO: 75 - DNA, 20 bp, Artificial Sequence
  Synthetic Construct
tcaggtagtcatggcagcag

(19) United States
(12) Patent Application Publication
(10) Pub. No.: US 2009/0280096 A1
(43) Pub. Date: Nov. 12, 2009

(54) PANCREATIC ENDOCRINE PROGENITOR CELLS DERIVED FROM PLURIPOTENT STEM CELLS

(76) Inventors: Atsushi KUBO, Osaka (JP); Kristina Bonham, South San Francisco, CA (US); Robert Stull, Alameda, CA (US); H. Ralph Snodgrass, San Mateo, CA (US)

Correspondence Address: MORRISON & FOERSTER LLP, 755 PAGE MILL RD, PALO ALTO, CA, 94304-1018, US

(21) Appl. No.: 12/464,005

(22) Filed: May 11, 2009

Related U.S. Application Data
(60) Provisional application No. 61/061,070, filed on Jun. 12, 2008.
(60) Provisional application No. 61/052,155, filed on May 9, 2008.

(51) Int. Cl. A61K 35/39; C12N 5/08; C12N 15/87; C12Q 1/02; A61P 3/10

(52) U.S. Cl. 424/93.7; 435/366; 435/455; 435/377; 435/29

(57) ABSTRACT

The invention provides pluripotent cells modified to overexpress Pdx1 and Ngn3. Pluripotent cells include embryonic stem cells and induced pluripotent stem cells. Methods of producing pancreatic endocrine progenitor cells from ES cells or from iPS cells by forced expression of Pdx1 and Ngn3 are provided. Pancreatic endocrine progenitor cells are useful for drug discovery and cell replacement therapy.

### CROSS-REFERENCE TO RELATED APPLICATIONS

This application claims benefit of U.S. Provisional Patent Application Ser. No. 61/052,155 filed May 9, 2008 and U.S. Provisional Patent Application Ser. No. 61/061,070 filed Jun. 12, 2008, each application is hereby incorporated by reference in their entirety.

### FIELD OF THE INVENTION

The field of this invention relates generally to pancreatic endocrine precursor cells derived from pluripotent stem cells including embryonic stem cells and induced pluripotent stem cells.

### BACKGROUND OF THE INVENTION

Directed differentiation of embryonic stem cells to therapeutically important cell types is a major focus of stem cell research. These differentiated cells have multiple applications, from translational medicine to modeling tissues in vitro. One important aspect of tissue modeling is the ability to use those tissues in lieu of animal models and/or transformed cells that may not have normal biological responses. This is particularly important in drug screening, where specific effects and potential byproducts and toxicities must be determined for thousands of compounds making direct in vivo screening intractable. Since these compounds will eventually be used in humans, an innovative and clinically predictive screening assay that takes advantage of human embryonic stem cell differentiation will be a significant improvement over current pharmaceutical methods (Klimanskaya, I et al 2008 Nat. Rev. Drug Dicover. 7:131-142).

The differentiation of embryonic stem cells to pancreatic endocrine progenitor cells is of particular interest in the development of therapies for the treatment of endocrine disorders such as diabetes. Pancreatic endocrine progenitor cells can be used in screening protocols in the development of drugs to induce the generation of insulin secreting cells. In other cases, pancreatic endocrine progenitor cells can be used in the development of cell therapies in the treatment of diabetes. Islet transplantation is under investigation for the treatment of type 1 diabetes patients and therapeutic progress towards insulin independence has been demonstrated (Shapiro, A. M. et al., 2000 N Engl J. Med. 343(4):230-238; Shapiro, A. M. et al. 2006 N Engl J. Med. 355(13):1318-1330). This approach, however, is limited by the shortage of transplantable islets. Alternative sources for β-cells are under investigation and include pancreatic duct cells and progenitors (Bonner-Weir, 2000 #4; (Seaberg, R. M. et al. 2004 Nat. Biotechnol. 22(9):1115-1124; Gershengorn, M. C. et al. 2004 Science 306:2261-2264). In this regard, embryonic stem (ES) cells are potentially useful to generate insulin producing cells because they are a renewable source of cells that retain the potential to differentiate into endoderm-derived tissues, such as pancreas (Smith, 2001; Keller, G. M. 1995 Curr Opin Cell Biol. 1995 7(6):862-869; Wells, 1999). Several groups have reported that definitive endoderm can be induced by activin A in mouse and human ES cells (Kubo, A. et al. 2004 Development 131:1651-1662; Tada, S. et al. 2005 Development 132(19):4363-4374; D'Amour, K. A. et al. 2005 Nat Biotechnol 23(12):1534-1541), US Patent Applications 2006/0003446 and 2006/0276420.

Another source of cells that are potentially useful to generate insulin producing cells is induced Pluripotent Stem (iPS) cells. Here, differentiated cells are reprogrammed to a pluripotent state. iPS cells are believed to have many aspects of natural pluripotent stem cells, such as embryonic stem cells, including the expression of certain stem cell genes and proteins, chromatin methylation patterns, doubling time, embryoid body formation, teratoma formation, viable chimera formation, and potency and differentiability. An example of differentiation of iPS cells into insulin-secreting islet-like cells is provided by Tateishi, K. et al. (2008) J. Biol. Chem.

In the embryo, the pancreas is derived from the epithelium in the foregut endoderm and forms dorsal and ventral buds at approximately embryonic day 9 (Habener, J. F. et al. 2005 Endocrinology 146(3):1025-1034; Murtaugh, L C and Melton, D A, 2003 Annu Rev Cell Dev Biol. 19:71-89). Sequential activation of transcriptional factors plays a critical role during pancreas and β-cell development (FIG. 1). Pdx1/Ipf1 is expressed in the embryonic duodenum which gives rise to the dorsal and ventral pancreas (Ohlsson, H. et al. 1993 EMBO J. 12(11):4251-4259; Leonard, J. et al. 1993 Mol. Endocrinol. 7(10):1275-1283; Miller, C. P. et al. 1994 EMBO J. 13(5):1145-1156). Pdx1 mutant mice show pancreatic agenesis after bud formation (Jonsson, J. et al. 1994 Nature 371(6498):606-609) and ectopic expression of Pdx1 induced cell budding from the gut epithelium (Grapin-Botton, A. et al., 2001 Genes Dev. 15(4):444-454). After pancreatic bud formation, Neurogenin3 (Ngn3) plays a critical role for pancreatic endocrine precursors. Mice lacking Ngn3 show defects in four pancreatic endocrine cells, producing insulin (Ins), glucagon (Gcg), somatostatin (Sst) and pancreatic polypeptide (Ppy) (Gradwohl, G. et al., 2000 Proc Natl Acad Sci USA. 97(4):1607-1611). Lineage tracking study using a Cre-ER loxP system has shown that Ngn3 positive cells give rise to these four pancreatic endocrine cells (Gu, G. et al. 2002 Development 129(10):2447-2457). Using targeted disruption of genes in mice, it has been shown that additional transcriptional factors such as Pax4 (Sosa-Pineda, B. et al., 1997 Nature 1997 386(6623):399-402), NeuroD (Naya, F. J. et al., 1997 Genes Dev. 11(18):2323-2334), Nkx×2.2 (Sussel, L. et al., 1998 Development 125(12):2213-2221), and Nkx×6.1 (Sander, M. et al. 2000 Development 127(24):5533-5540) are critical for specification from pancreatic endocrine progenitors to insulin producing cells (β-cells). These results demonstrate that critical factors must be expressed at each stage for the specification through gut endoderm, pancreatic bud, pancreatic endocrine progenitor and β-cell formations.

We have previously established a protocol for the development of definitive endoderm during mouse ES cell differentiation (Kubo, A. et al. 2004 Development 131:1651-1662; Gouon-Evans, V. et al. 2006 Nat. Biotechnol. 24(11):1402-1411). D'Amour et al. have reported that pancreatic hormone-expressing endocrine cells could be differentiated from human ES cell-derived endoderm induced by activin (D'Amour, K. A. et al. 2005 Nat Biotechnol 23(12):1534-1541; D'Amour, K. A. et al. 2006 Nat Biotechnol 24(11):1392-1401). These studies focused on elucidating soluble factors that participate in pancreas development during human ES cell differentiation and showed that the process mimics embryonic pancreas development from gut endoderm.

Other methods to produce islet cells from embryonic stem cells have been described; for example, U.S. Pat. Nos. 7,033,831 and 7,326,572; WO 2007/149182 and Jiang J et al. (2007) Stem Cells 25:1940-1953.

### BRIEF SUMMARY OF THE INVENTION

The invention provides pluripotent stem cells that are modified to overexpress Pdx1 and Ngn3. In some aspects of the invention, the pluripotent stem cells are embryonic stem (ES) cells. In some aspects of the invention, the pluripotent stem cells are induced Pluripotent Stem (iPS) cells. In some aspects of the invention, expression of Pdx1 and Ngn3 are under the control of one or more inducible promoters. In some aspects of the invention, overexpression of Pdx1 and Ngn3 is simultaneous and in some aspects of the invention overexpression of Pdx1 and Ngn3 is sequential. In some aspects of the invention, expression of Pdx1 and Ngn3 is under the control of the same inducible promoter. In some aspects, genes encoding Pdx1 and Ngn3 are linked by an internal ribosome entry site (IRES). In some aspects of the invention, expression of Pdx1 and Ngn3 are under the control of a tetracycline (tet) inducible promoter.

The invention also provides ES or iPS cells that are modified to overexpress Pdx1 and Ngn3 and further comprise a reporter molecule. In some aspects of the invention, the reporter molecule is operably linked to a promoter expressed in pancreatic endocrine progenitor cells or derivatives thereof but not expressed in primitive endoderm. In some aspects, expression of Pdx1 and Ngn3 are under the control of one or more inducible promoters. In some aspects, the reporter molecule is β-lactamase (BLA) and the gene encoding BLA is operably linked to a promoter expressed in pancreatic endocrine progenitor cells or derivatives thereof but not expressed in primitive endoderm. In some aspects, the bla gene is operably linked to an insulin promoter. In some aspects, the insulin promoter is the insulin 1 promoter.

The invention provides ES cells or iPS cells that are modified to overexpress Pdx1, Ngn3 and MafA. In some aspects of the invention, expression of Pdx1, Ngn3 and MafA are under the control of one or more inducible promoters. In some aspects of the invention, overexpression of Pdx1, Ngn3 and MafA is simultaneous and in some aspects of the invention overexpression of Pdx1, Ngn3 and MafA is sequential. In some aspects of the invention, expression of Pdx1 and Ngn3 are simultaneous followed by induction of expression of MafA. In some aspects of the invention, expression of Pdx1 and Ngn3 is under the control of the same inducible promoter and expression of MafA is under the control of a different promoter. In some aspects, genes encoding Pdx1 and Ngn3 are linked by an IRES. In some aspects, of the invention, expression of Pdx1 and Ngn3 are under the control of a tetracycline inducible promoter. In some aspects of the invention, ES or iPS cells modified to overexpress Pdx1, Ngn3 and MafA, further comprise a reporter molecule. In some aspects of the invention, the reporter molecule is operably linked to a promoter expressed in pancreatic endocrine progenitor cells, primitive beta-islet cells or derivatives thereof but not expressed in primitive endoderm.

The invention also provides methods of producing pluripotent stem cells to overexpress Pdx1 and Ngn3 by introducing one or more nucleic acids encoding Pdx1 and Ngn3 into the pluripotent stem cells. In some embodiments, the pluripotent stem cells are ES cells. In some embodiments, the pluripotent stem cells are iPS cells. In some aspects, genes encoding Pdx1 and said Ngn3 are operably linked to one or more inducible promoters. In some aspects, the invention provides methods of producing embryonic stem cells or iPS cells to overexpress Pdx1 and Ngn3 and to comprise a reporter molecule by introducing one or more nucleic acids encoding Pdx1, Ngn3 and the reporter molecule into the ES or iPS cells. In some aspects, the reporter molecule is operably linked to a promoter expressed in pancreatic endocrine progenitor cells or derivatives thereof but not expressed in primitive endoderm.

In some aspects, the invention provides methods of producing embryonic stem cells to overexpress Pdx1 and Ngn3 by introducing one or more nucleic acids encoding Pdx1 and Ngn3 into the ES cells and allowing the nucleic acids to integrate in the ES genome. In some aspects, genes encoding Pdx1 and Ngn3 are operably linked to one or more inducible promoters. In some aspects, the invention provides methods of producing embryonic stem cells to overexpress Pdx1 and Ngn3 and to comprise a reporter molecule by introducing one or more nucleic acids encoding Pdx1, Ngn3 and the reporter molecule or nucleic acid encoding the reporter molecule into the ES cells and allowing the nucleic acids to integrate into the ES genome. In some aspects, the reporter molecule is operably linked to a promoter expressed in pancreatic endocrine progenitor cells or derivatives thereof but not expressed in primitive endoderm. In some aspects, the Pdx1 and Ngn3 genes integrate into the HPRT locus or the ROSA26 locus. In some aspects, the reporter molecule or the gene encoding the reporter molecule integrates into the insulin locus.

In some aspects, the invention provides methods of producing iPS cells to overexpress Pdx1 and Ngn3 by introducing one or more nucleic acids encoding Pdx1 and Ngn3 into the iPS cells and allowing the nucleic acids to integrate in the iPS genome. In some aspects, genes encoding Pdx1 and Ngn3 are operably linked to one or more inducible promoters. In some aspects, the invention provides methods of producing iPS cells to overexpress Pdx1 and Ngn3 and to comprise a reporter molecule by introducing one or more nucleic acids encoding Pdx1, Ngn3 and the reporter molecule or nucleic acid encoding the reporter molecule into the iPS cells and allowing the nucleic acids to integrate into the iPS genome. In some aspects, the reporter molecule is operably linked to a promoter expressed in pancreatic endocrine progenitor cells or derivatives thereof but not expressed in primitive endoderm. In some aspects, the Pdx1 and Ngn3 genes integrate into the HPRT locus or the ROSA26 locus. In some aspects, the reporter molecule or the gene encoding the reporter molecule integrates into the insulin locus.

The invention provides methods of producing pluripotent stem cells to overexpress Pdx1, Ngn3 and MafA, by introducing one or more nucleic acids encoding Pdx1, Ngn3 and MafA into the cells. In some embodiments, the pluripotent stem cells are ES cells. In some embodiments, the pluripotent stem cells are iPS cells. The nucleic acids may be introduced at the same time or separately. In some aspects, the one or more nucleic acids encoding Pdx1, Ngn3 and MafA are operably linked to one or more inducible promoters. In some aspects, genes encoding Pdx1 and Ngn3 are operably linked to one inducible promoter. In some cases, genes encoding Pdx1 and Ngn3 are linked by an IRES. In some aspects, the invention provides methods of producing embryonic stem cells to overexpress Pdx1, Ngn3 and MafA and further comprise a reporter molecule. In some aspects, the invention provides methods of producing ES cells or iPS cells to overexpress Pdx1, Ngn3 and MafA and further comprise a reporter molecule. The reporter molecule may be introduced into the ES cells or iPS cells before, at the same time, or after introduction of the one or more nucleic acids encoding Pdx1, Ngn3 and MafA. In some aspects, the reporter molecule is operably linked to a promoter expressed in pancreatic endocrine progenitor cells or derivatives thereof but not expressed in primitive endoderm.

The invention provides methods of producing an embryonic stem cell to overexpress Pdx1, Ngn3 and MafA, by introducing one or more nucleic acids encoding Pdx1, Ngn3 and MafA into the cells and allowing the nucleic acids to integrate in the ES genome. In some aspects, the one or more nucleic acids encoding Pdx1, Ngn3 and MafA are operably linked to one or more inducible promoters. In some aspects, genes encoding Pdx1 and Ngn3 are operably linked to one inducible promoter. In some cases, genes encoding Pdx1 and Ngn3 are linked by an IRES. In some aspects, the invention provides methods of producing embryonic stem cells to overexpress Pdx1, Ngn3 and MafA and further comprise a reporter molecule. The reporter molecule may be introduced into the ES cells and allowed to integrate in the ES genome before, at the same time, or after introduction of the one or more nucleic acids encoding Pdx1, Ngn3 and MafA. In some aspects, the reporter molecule is operably linked to a promoter expressed in pancreatic endocrine progenitor cells or derivatives thereof but not expressed in primitive endoderm. In some aspects, the Pdx1, Ngn3 and MafA genes integrate into the HPRT locus or the ROSA26 locus. In some aspects, the reporter molecule or the gene encoding the reporter molecule integrates into the insulin locus.

The invention provides methods of producing an iPS cell to overexpress Pdx1, Ngn3 and MafA, by introducing one or more nucleic acids encoding Pdx1, Ngn3 and MafA into the cells and allowing the nucleic acids to integrate in the iPS genome. In some aspects, the one or more nucleic acids encoding Pdx1, Ngn3 and MafA are operably linked to one or more inducible promoters. In some aspects, genes encoding Pdx1 and Ngn3 are operably linked to one inducible promoter. In some cases, genes encoding Pdx1 and Ngn3 are linked by an IRES. In some aspects, the invention provides methods of producing iPS cells to overexpress Pdx1, Ngn3 and MafA and further comprise a reporter molecule. The reporter molecule may be introduced into the iPS cells and allowed to integrate in the iPS genome before, at the same time, or after introduction of the one or more nucleic acids encoding Pdx1, Ngn3 and MafA. In some aspects, the reporter molecule is operably linked to a promoter expressed in pancreatic endocrine progenitor cells or derivatives thereof but not expressed in primitive endoderm. In some aspects, the Pdx1, Ngn3 and MafA genes integrate into the HPRT locus or the ROSA26 locus. In some aspects, the reporter molecule or the gene encoding the reporter molecule integrates into the insulin locus.

The invention provides methods of producing pancreatic endocrine progenitor cells from pluripotent stem cells comprising the steps of (a) producing definitive endoderm cells from said pluripotent stem cells, (b) expressing Pdx1 and Ngn3 in said definitive endoderm cells, and (c) culturing the cells for sufficient time to identify pancreatic endocrine progenitor cells. In some embodiments, the pluripotent stem cells are embryonic stem cells. In some embodiments, the pluripotent stem cells are iPS cells. In some cases, the pancreatic endocrine progenitor cells are identified by expression of insulin; for example, by identification of insulin mRNA in cells overexpressing Pdx1 and Ngn3. In some embodiments, the method includes an additional step of culturing the pancreatic endocrine progenitor cells in a monolayer.

In some aspects, the invention provides methods of producing pancreatic endocrine progenitor cells from pluripotent stem cells comprising the steps of (a) producing definitive endoderm cells from pluripotent stem cells, (b) initiating expression of Pdx1 in the definitive endoderm cells, (c) analyzing the Pdx1-expressing cells for the expression of insulin mRNA, (d) initiating expression of Ngn3 in the Pdx1-expressing cells, and (e) culturing the said Pdx1/Ngn3-expressing cells for sufficient time to identify pancreatic endocrine progenitor cells. In some embodiments, the pluripotent stem cells are embryonic stem cells. In some embodiments, the pluripotent stem cells are iPS cells. In some cases, the pancreatic endocrine progenitor cells are identified by expression of insulin. In some embodiments, the method includes an additional step of culturing the pancreatic endocrine progenitor cells in a monolayer.

The invention provides methods of producing primitive beta-islet cells from pluripotent stem cells comprising the steps of (a) producing definitive endoderm cells from the pluripotent stem cells, (b) expressing Pdx1 and Ngn3 in the definitive endoderm cells, (c) culturing the Pdx1/Ngn3-expressing cells for sufficient time to identify pancreatic endocrine progenitor cells by measuring expression of insulin, (d) expressing MafA in the pancreatic endocrine progenitor cells, and (e) culturing the cells for sufficient time to identify primitive beta-islet cells by measuring secretion of insulin. In some embodiments, the pluripotent stem cells are embryonic stem cells. In some embodiments, the pluripotent stem cells are iPS cells. In some embodiments, the expression of Pdx1 and Ngn3 is simultaneous. In some embodiments of the inventions, the expression of Pdx1 and Ngn3 is sequential. In some aspects of the invention, the expression of Pdx1, Ngn3 and MafA is simultaneous. In some embodiments, the method includes an additional step of culturing the pancreatic endocrine progenitor cells in a monolayer.

The invention provides methods of producing pancreatic endocrine progenitor cells from pluripotent stem cells. In some embodiments, the pluripotent stem cells are embryonic stem cells. In some embodiments, the pluripotent stem cells are iPS cells. In some aspects, embryonic bodies (EB) are prepared from the pluripotent stem cell modified to express Pdx1 and Ngn3 under the control of an inducible promoter. Cells are dissociated and incubated in the presence of activin A to induce endoderm on about day 2. Cells are dissociated and expression of Pdx1 and Ngn3 is induced starting around days 4-6. Cells are plated on low attachment plates starting about days 6-9, and then cultured for sufficient time to identify pancreatic endocrine progenitor cells. In some aspects, cells are differentiated as monolayer cultures. In some aspects, the pluripotent cells are allowed to differentiate without forming EBs in step (a). In some cases, the resultant pancreatic endocrine progenitor cells are cultured in a monolayer. In some aspects of the invention, a nucleic acid encoding a reporter molecule is introduced to the cells prior to identifying pancreatic endocrine progenitor cells. In some embodiments, a nucleic acid encoding a reporter molecule is introduced to the cells on about days 4 to 6. In some embodiments, a nucleic acid encoding a reporter molecule is introduced to the cells on about days 4 to 9. In some embodiments, a nucleic acid encoding a reporter molecule is introduced to the cells on about days 6 to 9. In some embodiments, a nucleic acid encoding a reporter molecule is introduced to the cells on about three days prior to identifying pancreatic endocrine progenitor cells. In some embodiments, a nucleic acid encoding a reporter molecule is introduced to the cells for a sufficient time to allow expression of the reporter molecule in the pancreatic endocrine progenitor cell to allow identification of pancreatic endocrine progenitor cells. In some aspects, the pluripotent cells, modified to overexpress Pdx1 and Ngn3 are also modified to express a reporter molecule. In some cases, the reporter molecule is operably linked to a promoter expressed in pancreatic endocrine progenitor cells or derivatives thereof but not expressed in primitive endoderm. Expression of the reporter molecule under the pancreatic endocrine-related promoter can assist in identifying pancreatic endocrine progenitor cells.

The invention provides methods to produce primitive beta-islet cells from pluripotent stem cells. Similar methods may be used to produce pancreatic endocrine progenitor cells from ES cells or iPS cells by differentiating the ES cells or iPS cells to definitive endoderm followed by overexpression of Pdx1 and Ngn3 as described above. Nucleic acid encoding MafA is introduced to the pancreatic endocrine progenitor cells on about days 4 to 6 of differentiation to further differentiate the cells toward a beta-islet cell fate. In some embodiments, primitive beta-islet cells are identified by expression and/or secretion of insulin.

The invention provides methods of producing primitive beta-islet cells from pluripotent stem cells comprising the steps of (a) preparing embryonic bodies (EB) from the pluripotent stem cell modified to overexpress Pdx1, Ngn3 and MafA under the control of inducible promoters, (b) dissociating the cells and incubating the cells in the presence of activin A on about day 2, (c) dissociating the cells and inducing expression of Pdx1 and Ngn3 starting about day 4-day 6, (d) inducing expression of MafA, (e) plating the cells on low attachment plates about day 6-day 9, and (f) culturing the cells for sufficient time to identify primitive beta-islet cells. In some aspects, the pluripotent cells are allowed to differentiate without forming EBs in step (a). In some aspects of the invention, the pluripotent stem cells further comprise a reporter molecule that is operably linked to a promoter expressed in pancreatic endocrine progenitor cells or derivatives thereof but not expressed in primitive endoderm. Expression of the reporter molecule under the pancreatic endocrine-related promoter can assist in identifying primitive beta-islet cells or derivatives thereof. In some embodiments, the pluripotent stem cells are embryonic stem cells. In some embodiments, the pluripotent stem cells are iPS cells.

In some aspects, pancreatic endocrine progenitor cells are derived from pluripotent stem cells by culturing a population of cells modified to overexpress Pdx1 and Ngn3 on about day −4. Cells are passaged on about day −2 and then EBs are induced on about day 0. Cells are dissociated and incubated in the presence of activin A on about day 2. Cells are dissociated and expression of Pdx1 and Ngn3 is induced starting about days 4-6. Cells are plated starting on about day 6-day 9 and culturing the cells for sufficient time to identify pancreatic endocrine progenitor cells. In some aspects of the invention, cells are maintained as a monolayer throughout the differentiation process. In some aspects, the resulting pancreatic endocrine progenitor cells are cultured as a monolayer. In some aspects, the pluripotent cells, modified to overexpress Pdx1 and Ngn3 are also modified to express a reporter molecule. In some cases, the reporter molecule is operably linked to a promoter expressed in pancreatic endocrine progenitor cells or derivatives thereof but not expressed in primitive endoderm. Expression of the reporter molecule under the pancreatic endocrine-related promoter can assist in identifying pancreatic endocrine progenitor cells. In some embodiments, the pluripotent stem cells are embryonic stem cells. In some embodiments, the pluripotent stem cells are iPS cells.

In some aspects of the invention, primitive beta-islet cells are produced from pancreatic progenitor cells produced by the method described above. Nucleic acid encoding MafA is introduced to the cells on about days 4 to 6 to further differentiate the cells toward a beta-islet cell fate. In some embodiments, primitive beta-islet cells are identified by expression and/or secretion of insulin. In some embodiments, the pluripotent stem cells are embryonic stem cells. In some embodiments, the pluripotent stem cells are iPS cells.

The invention provides methods of producing primitive beta-islet cells from embryonic stem cells comprising the steps of (a) culturing a population of cells modified to overexpress Pdx1, Ngn3 and MafA to initiate differentiation on about day −4, (b) passaging the cells on about day −2, (c) preparing EBs from pluripotent stem cells on about day 0, (d) dissociating the cells and incubating the cells in the presence of activin A on about day 2, (e) dissociating the cells and inducing expression of Pdx1, Ngn3 and MafA in the cells starting about day 4-day 6, (f) plating the cells on about day 6-day 9, (g) culturing the cells for sufficient time to identify pancreatic endocrine progenitor cells. In some aspects, the pluripotent cells are allowed to differentiate without forming EBs in step (a). In some aspects of the invention, the pluripotent stem cells further comprise a reporter molecule that is operably linked to a promoter expressed in pancreatic endocrine progenitor cells or derivatives thereof but not expressed in primitive endoderm. Expression of the reporter molecule under the pancreatic endocrine-related promoter can assist in identifying primitive beta-islet cells or derivatives thereof. In some embodiments, the pluripotent stem cells are embryonic stem cells. In some embodiments, the pluripotent stem cells are iPS cells.

Methods of screening a compound or agent for its ability to modulate pancreatic endocrine cell function are provided. In some aspects, the compound or agent is combined with an pancreatic endocrine progenitor cell or primitive beta-islet cell of the invention and any phenotypic or metabolic changes in the cell that result from being combined with the compound are determined and correlated with an ability of the compound to modulate secretion of insulin, glucagon, gherlin, or somatostatin or proliferation of insulin secreting cells. In some aspects, the compound or agent is combined with a pancreatic endocrine progenitor cell or primitive beta-islet cell of the invention and cultured for varying amounts of time. Phenotypic or metabolic changes in the cell that result from being combined with the compound or agent are correlated with the time of culturing the cells. In some aspects, the pancreatic endocrine progenitor cells produced from ES cells or iPS cells by overexpression of Pdx1 and Ngn3 are isolated prior to combination with the compound or agent. In some aspects, the primitive beta-islet cells produced from ES cells or iPS cells by overexpression of Pdx1, Ngn3 and MafA are isolated prior to combination with the compound or agent. In some aspects of invention, the pancreatic endocrine progenitor cells produced from ES cells or iPS cells by overexpression of Pdx1 and Ngn3 are also modified to express a reporter molecule that is operably linked to a promoter expressed in pancreatic endocrine progenitor cells or derivatives thereof but not expressed in primitive endoderm. In some aspects of invention, the primitive beta-islet cells produced from ES cells or iPS cells by overexpression of Pdx1, Ngn3 and MafA are also modified to express a reporter molecule that is operably linked to a promoter expressed in pancreatic endocrine progenitor cells or derivatives thereof but not expressed in primitive endoderm. The effects of the compound or agent are elucidated by determining changes in expression of the reporter molecule.

The invention also provides methods of pancreatic cell therapy. Pancreatic endocrine progenitor cells derived from ES cells or iPS cells by overexpression of Pdx1 and Ngn3, or derivatives of pancreatic endocrine progenitor cells of the invention, are administered to a subject in need of such treatment. Likewise, primitive beta-islet cells derived from ES cells or iPS cells by overexpression of Pdx1, Ngn3, and MafA or derivatives of primitive beta-islet cells of the invention, are administered to a subject in need of such treatment.

The invention provides methods of pancreatic cell therapy comprising administering to a subject in need of such treatment a composition comprising pancreatic endocrine progenitor cells produced by the methods of the invention. In some aspects, the invention provides methods of pancreatic cell therapy comprising administering to a subject in need of such treatment a composition comprising primitive beta-islet cells produced by the methods of the invention. In some embodiments the cells are derived from ES cells. In some embodiments, the cells are derived from iPS cells. In some embodiments, the pancreatic endocrine progenitor cells or primitive beta-islet cells are autologous to the subject. In some embodiments, the pancreatic endocrine progenitor cells or primitive beta-islet cells are allogeneic to the subject.

The invention provides compositions comprising pancreatic endocrine progenitor cells produced by the methods of the invention. The invention also provides compositions comprising primitive beta-islet cells produced by the methods of the invention.

The invention provides uses of pancreatic endocrine progenitor cells produced by the methods of the invention in the manufacture of a medicament for treatment of an individual in need of pancreatic cell therapy. In some embodiments, the invention provides uses of pancreatic endocrine progenitor cells produced by the methods of the invention in the manufacture of a medicament for the treatment of a condition associated with deficiency of a pancreatic endocrine hormone. In some embodiments, the deficiency in a pancreatic hormone is a deficiency in insulin, glucagon, somatostatin, gherlin and/or pancreatic polypeptide. In some embodiments, the condition is associated with a deficiency in insulin; for example Type I diabetes or Type II diabetes.

In some aspects, the invention provides uses of primitive beta-islet cells produced by the methods of the invention, or their derivatives, in the manufacture of a medicament for treatment of an individual in need of pancreatic cell therapy. In some embodiments, the invention provides uses of primitive beta-islet cells produced by the methods of the invention in the manufacture of a medicament for the treatment of a condition associated with deficiency of a pancreatic endocrine hormone. In some embodiments, the deficiency in a pancreatic hormone is a deficiency in insulin. In some embodiments, the condition is Type I diabetes or Type II diabetes.

### BRIEF DESCRIPTION OF THE DRAWINGS

FIG. 1 shows transcription factors related to pancreatic differentiation.

FIG. 2 shows expression constructs used to overexpress Pdx1 and/or Ngn3 in ES cells. R26 is the ROSA26 promoter. rtTA is the reverse tetracycline transactivator. pA refers to polyadenylation sequences. HPRT is the hypoxanthine-guanine phosphoribosyltransferase gene. TetO is the tetracycline operator. PGK is the phosphoglycerate kinase promoter. Neo is the gene conferring resistance to neomycin. IRES is an internal ribosome entry site.

FIG. 3 shows pancreatic differentiation induced by Pdx1 and Ngn3 in SP conditions. (A, B) Tet-pdx1 ES cells were cultured in SP conditions. Pdx1 expression was induced with (Dox+) or without (Dox−) doxycycline (Dox) at day 6, and cells were harvested at indicated time points. A. Gene expression was analyzed by RT-PCR. B. Ins1 mRNA levels were quantified by real time PCR and normalized to the 18S mRNA levels. Without Dox (Dox−), open squares; With Dox (Dox+), closed circles. (C, D, E) Embryoid bodies (EBs) were differentiated for 6 days in SP conditions, trypsinized and resuspended as single cell suspensions. A pIRES2-EGFP vector was electroporated into cells and cells were reaggregated for 3 days. C. At day 8, EGFP was evaluated by FACS. D. pIRES2-EGFP vectors, without insert (GFP), or with Pax4, Nkx×6.1 and Ngn3 were electroporated into day 6 EBs. At day 9, reaggregated EBs were harvested and gene expression was analyzed by RT-PCR. E. Ins1 mRNA levels at day 9 were quantified by a real time PCR and normalized to the 18S mRNA levels. (F, G) Tet-pdx1/ngn3 ES cells were cultured in SP conditions. Pdx1 and Ngn3 expression was induced with (Dox+) or without Dox (Dox−) at day 6 and cells were harvested at the indicated time points. F. Gene expression was analyzed by RT-PCR. G. Ins1 mRNA levels were quantified by a real time PCR and normalized to the 18S mRNA levels. Without Dox (Dox−), open squares; With Dox (Dox+), closed circles.

FIG. 4 shows pancreatic differentiation induced by Pdx1 and Ngn3 in SFD conditions. Tet-pdx1/ngn3 ES cells were cultured in SFD conditions. Pdx1 and Ngn3 expression was induced with (Dox+) or without (Dox−) Dox after day 4 and cells were harvested at the indicated time points. (A, B) Ins1 mRNA levels were quantified by a real time PCR and normalized to the 18S mRNA levels. A. Day 4 EBs were trypsinized and reaggregated with (closed circles) or without BMP4 (open squares) for days 4-6. EBs were harvested at days 6 and 9. B. At day 6, EBs were replated on gelatin coated dishes and floating EBs were transferred to low-cluster dishes at day 7. Attached monolayer EBs (open bars) and floating EBs (closed bars) were harvested at day 9. (C, D) Floating EBs were cultured in SFD conditions with (closed circles) or without (open squares) Dox. Ins1 (C) or Ins2 (D) mRNA levels were quantified by a real time PCR and normalized to the 18S mRNA levels.

FIG. 5 shows a time course of pancreas-related gene expression in SFD conditions. Tet-pdx1/ngn3 ES cells were cultured in SFD conditions. Pdx1 and Ngn3 expression was induced with (Dox+) or without (Dox−) Dox after day 4, and cells were harvested at the indicated time points. Expression of pancreas-related genes was analyzed by RT-PCR. (A) Secretory proteins and liver/intestine related-genes. (B) Insulin processing genes and glucose sensing genes. (C) Pancreas related-transcriptional factors.

FIG. 6 shows optimization and characterization of pancreatic EBs in SFD conditions. Tet-pdx1/ngn3 ES cells were cultured in SFD conditions. Pdx1 and Ngn3 expression was induced with (Dox+) or without (Dox−) Dox after day 4, and cells were harvested at the indicated time points. (A) CXCR4/c-kit^{-/-} or CXCR4/c-kit^{+/+} cells were sorted in day 4 EBs by using a FACS sorter. Sorted cells were reaggregated and replated at day 6 on gelatin coated plates. EBs were harvested at day 9. Ins1 mRNA levels were quantified by real time PCR and normalized to the 18S mRNA levels. (B) N2 media was added to or omitted from the SFD media for days 0-14. B27, with or without retinoic acid (RA), was combined in SFD for days 0-4 and for day 4-14 (also +/−N2). Ins1 mRNA levels were quantified by real time PCR and normalized to the 18S mRNA levels. (C) Tet-pdx1/ngn3 ES cells were cultured in SFD condition without N2 and RA for 18 days. Cytoplasmic insulin was stained and analyzed by FACS. (D) Floating EBs were cultured in SFD without N2 and RA for 18 days, with or without Dox. EBs were incubated in SFD without N2 and RA for 24 hours and supernatants were harvested. C-peptide, glucagon and somatostatin were measured by RIA or EIA. (E) Floating EBs were cultured in SFD without N2 and RA for 19 days and then were unstimulated or stimulated with KCl (3 or 30 mM), glucose (20 mM), tolbutaminde (100 μM), Forskolin (10 μM) or IBMX (0.5 mM) in HKRB buffer for 1 hour. Supernatants were harvested and C-peptide was measured by RIA.

FIG. 7 shows immunofluorescence analysis of pancreatic EBs induced by Pdx1 and Ngn3. Tet-pdx1/ngn3 ES cells were cultured in SFD without N2 and RA. At day 16, EBs were replated on glass bottom dishes coated with matrigel. Replated EBs were stained with antibodies for the indicated pancreatic endocrine cell markers. Insulin was visualized by Cy3-conjugated secondary antibody (red, right column in rows 2-5) and the indicated markers were stained by FITC-conjugated secondary antibody (green, middle column rows 1-3). Nuclei were stained with DAPI (blue). Middle panel of row 4 shows staining for insulin and DAPI and the right panel of row 4 shows double staining of insulin and Pdx1. The middle panel of row 5 shows double staining of Ngn3 and DAPI and the right column of row 5 shows double staining of insulin and Ngn3. Merge images between insulin and secondary antibody and including DAPI stain are shown in the left column. Magnification of right panel for C-peptide and insulin (row 1) was used 1000×. Magnification for the left panel was 400×.

FIG. 8 shows the Tet-pdx1/ngn3-MafA expression construct. R26 is the ROSA26 promoter. rtTA is the reverse tetracycline transactivator. pA refers to polyadenylation sequences. TetO is the tetracycline operator. PGK is the phosphoglycerate kinase promoter. Neo is the gene conferring resistance to neomycin. IRES is an internal ribosome entry site.

FIG. 9 shows results of microarray analysis of insulin expression following overexpression of Pdx1, Ngn3 and MafA.

FIG. 10 shows a map of plasmid pUB/Bsd+3′ Ins1. 3′ arm designates a 3′ portion of the Ins1 gene. BSD designates a gene conferring resistance to blastidicidin. pUBC is the UbC promoter. Ampicillin-r refers to a gene conferring resistance to ampicillin. pUC ori is the origin of replication from pUC.

FIG. 11 shows a map of plasmid pUB/Bsd+3′+5′ Ins1. 3′ arm designates a 3′ portion of the ins1 gene and 5′ arm designates a 5′ portion of the ins1 gene. BSD designates a gene conferring resistance to blastidicidin. pUBC is the UbC promoter. Ampicillin-r refers to a gene conferring resistance to ampicillin. pUC ori is the origin of replication from pUC.

FIG. 12 shows a map of plasmid Ins1-Bla. 3′ arm designates a 3′ portion of the ins1 gene and 5′ arm designates a 5′ portion of the ins1 gene. Bla designates the β-lactamase gene. BSD designates a gene conferring resistance to blastidicidin. pUBC is the UbC promoter. Ampicillin-r refers to a gene conferring resistance to ampicillin. pUC ori is the origin of replication from pUC.

FIG. 13 shows a map of plasmid Ins 1-Bla2b. 3′ arm designates a 3′ portion of the ins1 gene and 5′ arm designates a 5′ portion of the ins1 gene. Bla designates the β-lactamase gene. BSD designates a gene conferring resistance to blastidicidin. pUBC is the UbC promoter. Ampicillin-r refers to a gene conferring resistance to ampicillin. pUC ori is the origin of replication from pUC. DTA designates the diphtheria toxin A gene under the control of a PGK promoter with an intervening sequence (IVS) and polyadenylation signal (polyA).

FIG. 14 shows a map of plasmid Ins1-Bla3b. 3′ arm designates a 3′ portion of the ins1 gene and 5′ arm designates a 5′ portion of the ins1 gene. Bla designates the β-lactamase gene. BSD designates a gene conferring resistance to blastidicidin. pUBC is the UbC promoter. Ampicillin-r refers to a gene conferring resistance to ampicillin. pUC ori is the origin of replication from pUC. DTA designates the diphtheria toxin A gene under the control of a PGK promoter with a polyadenylation signal (polyA).

FIG. 15 shows the genomic characterization of 673P and 673PN cells.

FIG. 16 shows detection of the 5′ arm of the target plasmid in ES cells.

FIG. 17 shows detection of the 3′ arm of the target plasmid in ES cells.

FIG. 18 shows induction of Pdx1 and Ngn3 by Dox in 673P and 673PN cells.

FIG. 19 shows immunocytochemistry of Dox-induced 673PN cells.

FIG. 20 demonstrates the sensitivity of the BLA assay.

FIG. 21 shows transient expression of pIns1-BLA3b in βTC6 cells.

FIG. 22 shows expression of BLA in mES-derived pancreas-like cells.

FIG. 23 shows construction of an insulin reporter cell line. A. Insertion of a GFP gene under the control of a brachyury promoter into the ROSA26 locus. B. Insertion of a tetracycline-regulatable gene expression system into the ROSA26 locus. C. Insertion of Tet-pdx1-IRES-ngn3 and Ins1-Bla into the ROSA26 locus.

FIG. 24 demonstrates mIns1 promoter-driven expression of BLA in 673 cells by fluorescence microscopy (A) and by Quantitation with a microplate reader (B).

FIG. 25 shows that Ins1 and BLA are induced in 673PN cells in response to introduction of MafA. Error bars show the range of fold change corresponding to one standard deviation.

### DETAILED DESCRIPTION OF THE INVENTION

### I. Introduction

The present invention relates, in part, to the transcriptional regulations that are critical to induce β-cell differentiation from ES cell-derived endoderm. For example, the combination of Pdx1 and Ngn3 induces pancreatic endocrine genes as well as β-cell-related transcriptional factors such as Pax4, Pax6, Isl1 and Nkx×2.2. Other pancreas-related proteins such, as C-peptide and insulin, can be detected by immunohistochemistry in these cells. In addition, these cells process and secrete insulin and respond to various insulin secretagogues.

The present invention provides pancreatic endocrine progenitor cells and methods for producing pancreatic endocrine progenitor cells from embryonic stem cells or from induced Pluripotent Stem (iPS) cells. The endocrine progenitor cells are useful to identify agents that modulate pancreatic endocrine function, to identify agents that affect cell growth and differentiation, to identify genes involved in pancreatic tissue development and to generate differentiated cells and tissues for cell replacement therapies.

The invention is based, in part, on the discovery that overexpression of Pdx1 and Ngn3 can induce differentiation of embryonic stem cell derived endoderm to a pancreatic endocrine cell fate. Forced expression of Pdx1 results in upregulation of pancreas-related genes such as insulin 1 (ins1) and insulin 2 (ins2) at day 20 of differentiation. Forced expression of Pdx1 and Ngn3 dramatically increases ins1 mRNA and at an earlier time, day 9, compared to Pdx alone. Forced expression of additional genes may further differentiation toward specific pancreatic endocrine cells. For, example, forced expression of Pdx1, Ngn3 and MafA may further induce differentiation of endoderm to a β cell lineage. As with embryonic stem cell derived endoderm, Pdx1 and Ngn3 overexpression may induce differentiation of iPS cell derived endoderm to a pancreatic endocrine cell fate.

The present invention provides embryonic stem cells modified to overexpress Pdx1 and Ngn3. In some aspects, the invention provides iPS cells modified to overexpress Pdx1 and Ngn3. Expression of Pdx1 and Ngn3 may be simultaneous or expression of Pdx1 and Ngn3 may be sequential. In some aspects of the invention, Pdx1 and Ngn3 are under the control of one or more inducible promoters. The use of inducible promoters may facilitate the temporal expression of Pdx1 and Ngn3 in ES cells or iPS cells. For example, before differentiation into endoderm, it may be desired to minimize expression of Pdx1 and Ngn3. Inducible promoters generally exhibit low activity in the absence of inducer. Following differentiation of ES cells or iPS cells to endoderm, overexpression of Pdx1 and Ngn3 may be induced to direct differentiation of the endoderm to a pancreatic endocrine progenitor fate. Timing of induction of Pdx1 and Ngn3 can be used to optimize differentiation of endoderm to pancreatic endocrine progenitor cells.

In some aspects of the invention, Pdx1 may be under the control of one inducible promoter and Ngn3 may be under the control of a different inducible promoter. In this case, expression of Pdx1 and Ngn3 may be controlled temporally relative to one another by controlled induction of the different inducible promoters. In some aspects of the invention, Pdx1 and Ngn3 are under the control of the same inducible promoter. In this case, the pdx1 and ngn3 genes may be linked in an expression cassette. For example, the pdx1 and ngn3 genes can be linked in one expression cassette through the use of an Internal Ribosome Entry Site (IRES). In some aspects, the invention provides ES cells modified with a pdx1-IRES-ngn3 expression cassette operably linked to a tetracycline-inducible promoter. In some cases, a Tet-pdx1-IRES-ngn3 expression cassette is stably introduced into the ES cells. In some cases, a Tet-pdx1-IRES-ngn3 expression cassette is transiently introduced into ES cells.

The invention provides ES cells modified to express a reporter molecule used to monitor differentiation of ES cells to pancreatic endocrine progenitor cells. In some aspects, the invention provides iPS cells modified to express a reporter molecule used to monitor differentiation of iPS cells to pancreatic endocrine progenitor cells. The reporter molecule is operably linked to a promoter that is expressed in pancreatic endocrine progenitor cells or derivatives thereof but not expressed in primitive endoderm. In some aspects of the invention, the reporter molecule is β-lactamase (BLA). In some aspects of the invention, the promoter expressed in pancreatic endocrine progenitor cells or derivatives thereof but not expressed in primitive endocrine cells is the promoter controlling the expression of a pancreatic endocrine hormone. For example, the promoter may be, but is not limited to, an insulin 1 promoter, an insulin 2 promoter, a glucagon promoter, a somatostatin promoter, a pancreatic polypeptide promoter and a ghrelin/obestatin preprohormone promoter. In some aspects of the invention, ES cells are modified to express BLA under the control of the ins1 promoter. In some cases, an Ins1-BLA expression cassette is stably introduced into the ES cells. In some cases, an Ins1-BLA expression cassette is transiently introduced into ES cells.

The invention provides ES cells or iPS cells that are modified to overexpress Pdx1, Ngn3 and MafA. Expression of Pdx1, Ngn3 and MafA may be simultaneous or expression of Pdx1, Ngn3 and MafA may be sequential. In some aspects of the invention, Pdx1, Ngn3 and MafA are under the control of one or more inducible promoters. Timing of induction of Pdx1, Ngn3 and MafA can be used to optimize differentiation of endoderm to pancreatic endocrine progenitor cells and to primitive beta-islet cells. In some aspects of the invention, Pdx1, Ngn3 and MafA may be under the control of different inducible promoters. In this case, expression of Pdx1, Ngn3 and MafA may be controlled temporally relative to one another by controlled activation of the different inducible promoters. In some aspects of the invention, Pdx1 and Ngn3 are under the control of the same inducible promoter, as described above, and MafA is under the control of a different promoter. In some cases, expression of MafA is controlled by an inducible promoter. In some cases, MafA is controlled by a constitutive promoter. In some aspects, the invention provides ES cells or iPS cells modified to overexpress Pdx1, Ngn3 and MafA and modified to express a reporter molecule under the control of a promoter expressed in pancreatic endocrine progenitor cells or derivatives thereof but not expressed in primitive endoderm.

The invention provides methods to produce embryonic stem cells modified to overexpress Pdx1 and Ngn3. In some aspects of the invention, nucleic acid encoding pdx1 and ngn3 genes are introduced into ES cells. In some cases the nucleic acids encoding pdx1 and ngn3 genes are stably introduced into the ES cells. In some cases the nucleic acid encoding pdx1 and ngn3 genes are transiently introduced into the ES cells. In some aspects, the invention provides methods to produce ES cells modified to overexpress Pdx1 and Ngn3 where the pdx1 and ngn3 genes are integrated into the ES genome. In some cases, the pdx1 and ngn3 genes are targeted to specific sites in the ES genome. For example, the pdx1 and ngn3 genes may be targeted to the HPRT locus or to the ROSA26 locus. Targeting can be accomplished using methods known in the art; for example, homologous recombination or through the use of a cre-lox recombination system.

In some aspects, the invention provides methods to produce embryonic stem cells modified to overexpress Pdx1, Ngn3 and MafA. In some aspects of the invention, nucleic acid encoding pdx1, ngn3 and mafA genes are introduced into ES cells. In some cases, the nucleic acids encoding one or more of pdx1, ngn3 and mafA genes are stably introduced into the ES cells. In some cases, the nucleic acids encoding one or more of pdx1, ngn3 and mafA genes are transiently introduced into the ES cells. In some aspects, the invention provides methods to produce ES cells modified to overexpress Pdx1, Ngn3 and MafA where the pdx1, ngn3 and mafA genes are integrated into the ES genome. In some cases, the pdx1, ngn3 and mafA genes are targeted to specific sites in the ES genome. For example, the pdx1, ngn3 and mafA genes may be targeted to the HPRT locus or to the ROSA26 locus. Targeting can be accomplished using methods known in the art; for example, homologous recombination or through the use of a cre-lox recombination system.

The invention provides methods to produce iPS cells modified to overexpress Pdx1 and Ngn3. In some aspects of the invention, nucleic acid encoding pdx1 and ngn3 genes are introduced into iPS cells. In some cases the nucleic acids encoding pdx1 and ngn3 genes are stably introduced into the iPS cells. In some cases, nucleic acids encoding pdx1 and ngn3 genes are introduced to differentiated cells before induction to pluripotent stem cells. In some cases, nucleic acids encoding pdx1 and ngn3 are introduced to iPS cells after reprogramming of differentiated cells. In some cases, nucleic acids encoding pdx1 and ngn3 are introduced to cells during the reprogramming process. In some cases the nucleic acid encoding pdx1 and ngn3 genes are transiently introduced into the iPS cells. In some aspects, the invention provides methods to produce iPS cells modified to overexpress Pdx1 and Ngn3 where the pdx1 and ngn3 genes are integrated into the iPS genome. In some cases, the pdx1 and ngn3 genes are targeted to specific sites in the iPS genome. Targeting can be accomplished using methods known in the art; for example, homologous recombination or through the use of a cre-lox recombination system.

In some aspects, the invention provides methods to produce iPS cells modified to overexpress Pdx1, Ngn3 and MafA. In some aspects of the invention, nucleic acid encoding pdx1, ngn3 and mafA genes are introduced into iPS cells. In some cases, the nucleic acids encoding one or more of pdx1, ngn3 and mafA genes are stably introduced into the iPS cells. In some cases, nucleic acids encoding pdx1, ngn3 and mafA genes are introduced to differentiated cells before induction to pluripotent stem cells. In some cases, nucleic acids encoding pdx1, ngn3 and mafA are introduced to iPS cells after reprogramming of differentiated cells. In some cases, nucleic encoding pdx1 and ngn3 and mafA are introduced to cells during the reprogramming process. In some cases, the nucleic acids encoding one or more of pdx1, ngn3 and mafA genes are transiently introduced into the iPS cells. In some aspects, the invention provides methods to produce iPS cells modified to overexpress Pdx1, Ngn3 and MafA where the pdx1, ngn3 and mafA genes are integrated into the iPS genome. In some cases, the pdx1, ngn3 and mafA genes are targeted to specific sites in the iPS genome. Targeting can be accomplished using methods known in the art; for example, homologous recombination or through the use of a cre-lox recombination system.

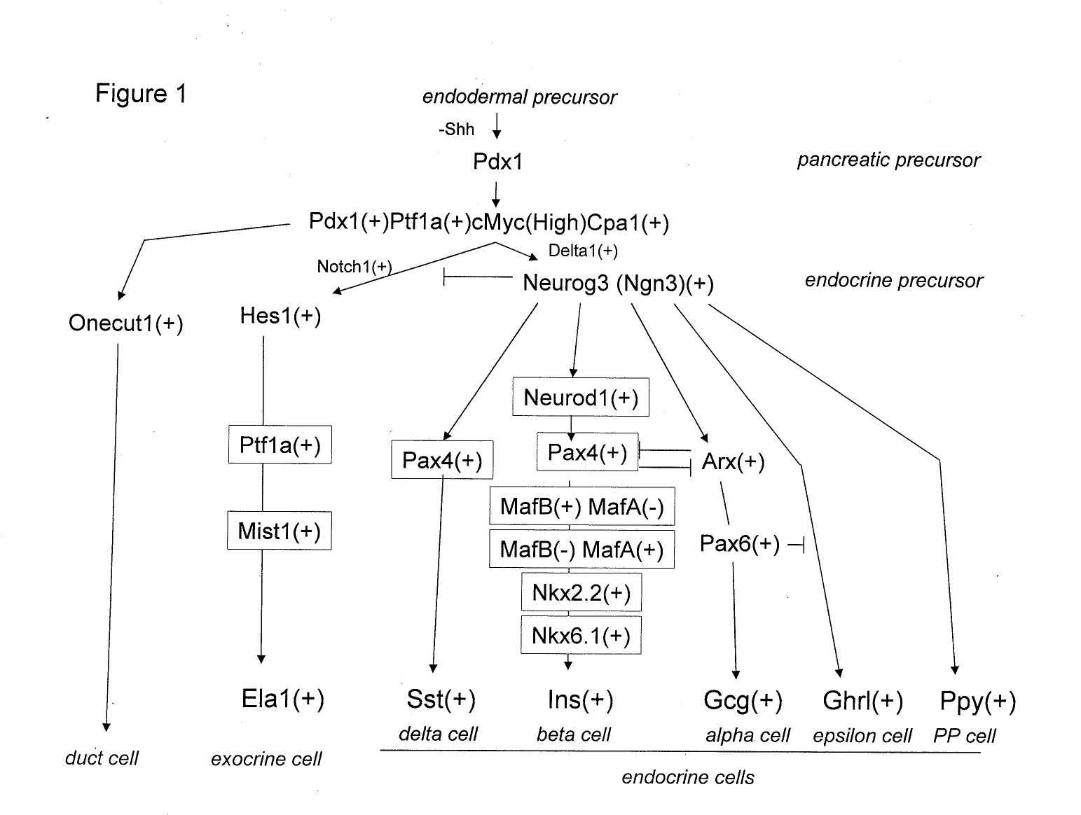

The invention provides methods to generate pancreatic endocrine progenitor cells and derivatives of pancreatic progenitor cells by forced expression of Pdx1 and Ngn3 in endoderm. A generalized scheme of differentiation of an endoderm precursor cells (e.g. definitive endoderm) to a variety of pancreatic cells in provided in FIG. 1. In some aspects of the invention, pluripotent cells such as ES cells or iPS cells are induced to form definitive endoderm. Overexpression of Pdx1 may lead to the formation of pancreatic progenitor cells. Overexpression of Pdx1 and Ngn3 may lead to the formation of pancreatic endocrine progenitor cells. Pancreatic endocrine progenitor cells may differentiate into cells secreting pancreatic endocrine hormones following expression of genes associated with a particular differentiation pathway. For example, overexpression of MafA in pancreatic endocrine progenitor cells may lead to the generation of primitive beta-islet cells.

The invention provides methods of producing pancreatic endocrine progenitor cells from embryonic stem cells. In some aspects, ES cells are first allowed to begin differentiation. Cells are then induced to form definitive endoderm. In some cases, cells are induced to form definitive endoderm by incubating cells in the presence of activin A. Pancreatic endocrine progenitor cells are then induced by overexpression of Pdx1 and Ngn3. In some cases, pancreatic endocrine progenitor cells and/or primitive beta-islet cells are induced by overexpression of Pdx1, Ngn3 and MafA. In some aspects of the invention, Pdx1 and Ngn3 are overexpressed transiently by introducing nucleic acids encoding pdx1 and ngn3 genes to endoderm cells. In some aspects of the invention, pdx1 and ngn3 genes are stably integrated into ES cells under the control of an inducible promoter and overexpression is induced by activation of the inducible promoter. In some aspects of the invention, Pdx1, Ngn3 and MafA are overexpressed transiently by introducing nucleic acids encoding pdx1, ngn3 and mafA genes to endoderm cells. In some aspects of the invention, pdx1, ngn3 and mafA genes are stably integrated into ES cells under the control of an inducible promoter and overexpression is induced by activation of the inducible promoter. In some aspects of the invention, pdx1 and ngn3 are integrated into ES cells under the control of an inducible promoter and mafA is transiently overexpressed. In some aspects of the invention, the ES cells further comprise a reporter molecule operably linked to a promoter active in pancreatic endocrine progenitor cells, primitive beta-islet cells or derivatives thereof but not expressed in primitive endoderm. In some cases, the reporter molecule is BLA and the pancreatic endocrine-specific promoter an ins1 promoter. In some aspects of the invention, the progression of ES cells to pancreatic endocrine progenitor cells can be monitored by expression of a reporter molecule operably linked to a promoter active in pancreatic endocrine progenitor cells or derivatives thereof but not expressed in primitive endoderm.

The invention provides methods of producing pancreatic endocrine progenitor cells from embryonic stem cells. In some aspects, ES cells are first induced to form EBs. EBs are then induced to form definitive endoderm. In some cases, EBs are induced to form definitive endoderm by incubating EB cells in the presence of activin A. Pancreatic endocrine progenitor cells are then induced by overexpression of Pdx1 and Ngn3. In some cases, pancreatic endocrine progenitor cells and/or primitive beta-islet cells are induced by overexpression of Pdx1, Ngn3 and MafA. In some aspects of the invention, Pdx1 and Ngn3 are overexpressed transiently by introducing nucleic acids encoding pdx1 and ngn3 genes to endoderm cells. In some aspects of the invention, pdx1 and ngn3 genes are stably integrated into ES cells under the control of an inducible promoter and overexpression is induced by activation of the inducible promoter. In some aspects of the invention, Pdx1, Ngn3 and MafA are overexpressed transiently by introducing nucleic acids encoding pdx1, ngn3 and mafA genes to endoderm cells. In some aspects of the invention, pdx1, ngn3 and mafA genes are stably integrated into ES cells under the control of an inducible promoter and overexpression is induced by activation of the inducible promoter. In some aspects of the invention, pdx1 and ngn3 are integrated into ES cells under the control of an inducible promoter and mafA is transiently overexpressed. In some aspects of the invention, the ES cells further comprise a reporter molecule operably linked to a promoter active in pancreatic endocrine progenitor cells or derivatives thereof but not expressed in primitive endoderm. In some cases, the reporter molecule is BLA and the pancreatic endocrine-specific promoter is an ins1 promoter. In some aspects of the invention, the progression of ES cells to pancreatic endocrine progenitor cells can be monitored by expression of a reporter molecule operably linked to a promoter active in pancreatic endocrine progenitor cells or derivatives thereof but not expressed in primitive endoderm.

In some aspects, the invention provides methods of producing pancreatic endocrine progenitor cells from embryonic stem cells in monolayer. ES cells are induced to form definitive endoderm. In some cases, ES cells are induced to form definitive endoderm by incubating ES cells in the presence of activin A. Pancreatic endocrine progenitor cells are then induced by overexpression of Pdx1 and Ngn3. In some cases, pancreatic endocrine progenitor cells are induced by overexpression of Pdx1, Ngn3 and MafA. In some aspects of the invention, Pdx1 and Ngn3 are overexpressed transiently by introducing nucleic acids encoding pdx1 and ngn3 genes to endoderm cells. In some aspects of the invention, pdx1 and ngn3 genes are stably integrated into ES cells under the control of an inducible promoter and overexpression is induced by activation of the inducible promoter. In some aspects of the invention, Pdx1, Ngn3 and MafA are overexpressed transiently by introducing nucleic acids encoding pdx1, ngn3 and mafA genes to endoderm cells. In some aspects of the invention, pdx1, ngn3 and mafA genes are stably integrated into ES cells under the control of an inducible promoter and overexpression is induced by activation of the inducible promoter. In some aspects of the invention, pdx1 and ngn3 are integrated into ES cells under the control of an inducible promoter and mafA is transiently overexpressed. In some aspects of the invention, the ES cells further comprise a reporter molecule operably linked to a promoter active in pancreatic endocrine progenitor cells or derivatives thereof but not expressed in primitive endoderm. In some cases, the reporter molecule is BLA and the pancreatic endocrine-specific promoter is an ins1 promoter. In some aspects of the invention, the progression of ES cells to pancreatic endocrine progenitor cells can be monitored by expression of a reporter molecule operably linked to a promoter active in pancreatic endocrine progenitor cells or derivatives thereof but not expressed in primitive endoderm. In some aspects of the invention, the progression of iPS cells to pancreatic endocrine progenitor cells can be monitored by expression of a reporter molecule operably linked to a promoter active in pancreatic endocrine progenitor cells or derivatives thereof but not expressed in primitive endoderm.

The invention provides methods of producing pancreatic endocrine progenitor cells from iPS cells. In some aspects, iPS cells are first allowed to begin differentiation. Cells are then induced to form definitive endoderm. In some cases, cells are induced to form definitive endoderm by incubating cells in the presence of activin A. Pancreatic endocrine progenitor cells are then induced by overexpression of Pdx1 and Ngn3. In some cases, pancreatic endocrine progenitor cells are induced by overexpression of Pdx1, Ngn3 and MafA. In some aspects of the invention, Pdx1 and Ngn3 are overexpressed transiently by introducing nucleic acids encoding pdx1 and ngn3 genes to endoderm cells. In some aspects of the invention, pdx1 and ngn3 genes are stably integrated into iPS cells under the control of an inducible promoter and overexpression is induced by activation of the inducible promoter. In some aspects of the invention, Pdx1, Ngn3 and MafA are overexpressed transiently by introducing nucleic acids encoding pdx1, ngn3 and mafA genes to endoderm cells. In some aspects of the invention, pdx1, ngn3 and mafA genes are stably integrated into iPS cells under the control of an inducible promoter and overexpression is induced by activation of the inducible promoter. In some aspects of the invention, pdx1 and ngn3 are integrated into iPS cells under the control of an inducible promoter and mafA is transiently overexpressed. In some aspects of the invention, the iPS cells further comprise a reporter molecule operably linked to a promoter active in pancreatic endocrine progenitor cells or derivatives thereof but not expressed in primitive endoderm. In some cases, the reporter molecule is BLA and the pancreatic endocrine-specific promoter an ins1 promoter. In some aspects of the invention, the progression of iPS cells to pancreatic endocrine progenitor cells can be monitored by expression of a reporter molecule operably linked to a promoter active in pancreatic endocrine progenitor cells or derivatives thereof but not expressed in primitive endoderm.

The invention provides methods of producing pancreatic endocrine progenitor cells from iPS cells. In some aspects, iPS cells are first induced to form EBs. EBs are then induced to form definitive endoderm. In some cases, EBs are induced to form definitive endoderm by incubating EB cells in the presence of activin A. Pancreatic endocrine progenitor cells are then induced by overexpression of Pdx1 and Ngn3. In some cases, pancreatic endocrine progenitor cells are induced by overexpression of Pdx1, Ngn3 and MafA. In some aspects of the invention, Pdx1 and Ngn3 are overexpressed transiently by introducing nucleic acids encoding pdx1 and ngn3 genes to endoderm cells. In some aspects of the invention, pdx1 and ngn3 genes are stably integrated into iPS cells under the control of an inducible promoter and overexpression is induced by activation of the inducible promoter. In some aspects of the invention, Pdx1, Ngn3 and MafA are overexpressed transiently by introducing nucleic acids encoding pdx1, ngn3 and mafA genes to endoderm cells. In some aspects of the invention, pdx1, ngn3 and mafA genes are stably integrated into iPS cells under the control of an inducible promoter and overexpression is induced by activation of the inducible promoter. In some aspects of the invention, pdx1 and ngn3 are integrated into iPS cells under the control of an inducible promoter and mafA is transiently overexpressed. In some aspects of the invention, the iPS cells further comprise a reporter molecule operably linked to a promoter active in pancreatic endocrine progenitor cells or derivatives thereof but not expressed in primitive endoderm. In some cases, the reporter molecule is BLA and the pancreatic endocrine-specific promoter an ins1 promoter. In some aspects of the invention, the progression of iPS cells to pancreatic endocrine progenitor cells can be monitored by expression of a reporter molecule operably linked to a promoter active in pancreatic endocrine progenitor cells or derivatives thereof but not expressed in primitive endoderm.

In some aspects, the invention provides methods of producing pancreatic endocrine progenitor cells from iPS cells in monolayer. iPS cells are induced to form definitive endoderm. In some cases, iPS cells are induced to form definitive endoderm by incubating iPS cells in the presence of activin A. Pancreatic endocrine progenitor cells are then induced by overexpression of Pdx1 and Ngn3. In some cases, pancreatic endocrine progenitor cells are induced by overexpression of Pdx1, Ngn3 and MafA. In some aspects of the invention, Pdx1 and Ngn3 are overexpressed transiently by introducing nucleic acids encoding pdx1 and ngn3 genes to endoderm cells. In some aspects of the invention, pdx1 and ngn3 genes are stably integrated into iPS cells under the control of an inducible promoter and overexpression is induced by activation of the inducible promoter. In some aspects of the invention, Pdx1, Ngn3 and MafA are overexpressed transiently by introducing nucleic acids encoding pdx1, ngn3 and mafA genes to endoderm cells. In some aspects of the invention, pdx1, ngn3 and mafA genes are stably integrated into iPS cells under the control of an inducible promoter and overexpression is induced by activation of the inducible promoter. In some aspects of the invention, pdx1 and ngn3 are integrated into iPS cells under the control of an inducible promoter and mafA is transiently overexpressed. In some aspects of the invention, the iPS cells further comprise a reporter molecule operably linked to a promoter active in pancreatic endocrine progenitor cells or derivatives thereof but not expressed in primitive endoderm. In some cases, the reporter molecule is BLA and the pancreatic endocrine-specific promoter is an ins1 promoter. In some aspects of the invention, the progression of iPS cells to pancreatic endocrine progenitor cells can be monitored by expression of a reporter molecule operably linked to a promoter active in pancreatic endocrine progenitor cells or derivatives thereof but not expressed in primitive endoderm. In some aspects of the invention, the progression of iPS cells to pancreatic endocrine progenitor cells can be monitored by expression of a reporter molecule operably linked to a promoter active in pancreatic endocrine progenitor cells or derivatives thereof but not expressed in primitive endoderm.

The present invention provides methods of screening compounds for their ability to modulate pancreatic endocrine cell function. Test compounds are contacted with pancreatic endocrine progenitor cells prepared from ES cells or iPS cells by overexpressing Pdx1 and Ngn3 and determining any phenotypic or metabolic changes in the cell that result from being combined with the compound, and correlating the change with an ability of the compound to modulate secretion of pancreatic endocrine hormones; for example, but not limited to, insulin, glucagon, gherlin, or somatostatin. In some cases, pancreatic endocrine progenitor cells and/or primitive beta-islet cells produced from ES cells or iPS cells by overexpression of Pdx1, Ngn3 and MafA are used to screen compounds for their ability to modulate pancreatic endocrine function.

In some aspects, the present invention provides methods of screening genes for their ability to modulate pancreatic endocrine cell function. Candidate genes may be identified by microarray analysis of pancreatic endocrine progenitor cells prepared from ES cells or iPS cells by overexpressing Pdx1 and Ngn3. The genes of interest are introduced into pancreatic endocrine progenitor cells prepared from ES cells or iPS cells by overexpressing Pdx1 and Ngn3 and determining any phenotypic or metabolic changes in the cell that result from overexpression of the candidate gene. Phenotypic or metabolic changes may be correlated the change with an ability of the cell to secrete pancreatic endocrine hormones; for example, but not limited to, insulin, glucagon, gherlin, or somatostatin.

In some aspects, the invention provides methods of screening compounds for their ability to modulate pancreatic endocrine cell function using a reporter cell system. Test compounds are contacted with pancreatic endocrine progenitor cells prepared from ES cells or iPS cells by overexpressing Pdx1 and Ngn3, and comprising a reporter molecule operably linked to a promoter active in pancreatic endocrine progenitor cells or derivatives thereof but not expressed in primitive endoderm. The ability of test compounds to modulate pancreatic endocrine cell function is assessed by determining changes in expression of the reporter molecule. In some cases, pancreatic endocrine progenitor cells and/or primitive beta-islet cells produced from ES cells or iPS cells by overexpression of Pdx1, Ngn3 and MafA are used to screen compounds for their ability to modulate pancreatic endocrine function.

The invention provides methods of pancreatic cell therapy comprising administering to a subject in need of such treatment a composition comprising pancreatic endocrine progenitor cells prepared from ES cells or iPS cells by overexpressing Pdx1 and Ngn3. In some cases, the invention provides methods of pancreatic cell therapy comprising administering to a subject in need of such treatment a composition comprising primitive beta-islet cells prepared from ES cells or iPS cells by overexpressing Pdx1, Ngn3 and MafA.

### II. General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987, and periodic updates); “PCR: The Polymerase Chain Reaction”, (Mullis et al., eds., 1994); “Current Protocols in Immunology” (J. E. Coligan et al., eds., 1991); “Stem Cell Culture” in Methods of Cell Biology, Vol. 86 (J. P. Mather, ed. 2008).

A “regulatory sequence” refers to any or all of the DNA sequences that controls gene expression. Examples of regulatory sequences include promoters, positive regulatory elements such as enhancers or DNA-binding sites for transcriptional activators, negative regulatory elements such as DNA-binding sites for a transcriptional repressors and insulators. Regulatory sequences may be found within, 5′ and/or 3′ to the coding region of the gene.

A “reporter,” “reporter gene,” “reporter molecule,” “reporter sequence,” “marker,” “marker gene” or “marker sequence”, used interchangeably herein, refers to a polynucleotide sequence whose expression product, reporter, or marker, (whether transcription and/or translation) can be detected by methods known in the art and described herein. Detection may be by any means, including but not limited to visible to the naked eye, spectroscopic, photochemical, biochemical, immunochemical, or chemical means.

As used herein, the term “totipotent cell” refers to a cell capable of developing into all lineages of cells. Similarly, the term “population of totipotent cells” refers to a composition of cells capable of developing into all lineages of cells. Also as used herein, the term “pluripotent cell” refers to a cell capable of developing into a variety (albeit not all) lineages. A “population of pluripotent cells” refers to a composition of cells capable of developing into less than all cell lineages. As such, a totipotent cell or composition of cells is less developed than a pluripotent cell or composition of cells. “Multipotent cells” are more differentiated relative to pluripotent cells, but are not terminally differentiated. As used herein, the terms “develop,” “differentiate,” and “mature” all refer to the progression of a cell from the stage of having the potential to differentiate into at least two different cellular lineages to becoming a specialized cell. Such terms can be used interchangeably for the purposes of the present application.

### II. Inducible Promoters

Inducible or regulatable promoters generally exhibit low activity in the absence of the inducer, and are up-regulated in the presence of the inducer. The inducible promoter can be induced by a molecule (e.g. a small molecule or protein) heterologous to the cell in which the expression cassette is to be used. A variety of inducible promoters are well-known to those of ordinary skill in the art. In some aspects of the invention, genes encoding Pdx1 and/or Ngn3 are operably linked to a tetracycline-inducible promoter. In some cases, genes encoding Pdx1 and Ngn3 are linked by an internal ribosome entry site (IRES) and are operably linked to a tetracycline-inducible promoter. Multicistronic and inducible expression systems are known in the art. See, for example, Chappell, S. A. et al. (2004) Proc Natl Acad Sci USA. 101(26):9590-9594; Goverdhana, S et al. (2005) Mol. Ther. 12:189-211; Hasegawa, K. et al. (2007) Stem Cells 25(7):1707-1712; and Vilaboa, N. and Voellmy, R. (2006) Curr. Gene Ther. 6:421-438.

### III. Reporter Molecules

Reporter molecules of the invention are known in the art. Recombinant DNA reporter gene systems were developed to enable quantitative, rapid and inexpensive measurement of the activity of the study of transcriptional promoters and enhancers (transcriptional regulatory elements, or TREs) that regulate the transcription of genes. In these procedures the coding regions of a molecularly cloned gene were replaced using recombinant DNA technology by a heterologous DNA sequence termed a reporter gene encoding a reporter protein. This reporter gene directs synthesis of an easily measurable reporter protein. Many different reporter proteins have successfully been used. Usually the protein is not found in the host cell type and the quantity of protein present can conveniently be measured. Recombinant DNAs encoding enzyme are often used as reporter genes due to the sensitivity of enzyme assays. Examples of enzymes used as reporter genes include chloramphenicol acetyltransferase (CAT; Gorman C M et al., (1982) Mol. Cell. Biol. 2:1044), beta-galactosidase (β-gal), beta-lactamase (BLA) Zlorkanik G, et al., (1998) Science 279:84-88), secreted alkaline phosphatase (SEAP; Berger J et al, (1988) Gene 66:1-10), and beta-glucuronidase (GUS) Jefferson R A, et al., (1987) EMBO J. 6:3901-3907). A number of luciferases (LUC) have been described including those from fireflies (De Wet J R, et al., (1987) Mol. Cell. Biol. 7:725-737), Renilla (Lorenz M M, et al., (1996) J. Biolumin. Chemilumin. 11:31-37) and Gaussia (Verhaegent M and Christopoulos T K (2002) Anal. Chem., 74, 4378-4385). In addition to enzymes, fluorescent proteins have found wide use as reporters for gene expression. The most commonly used fluorescent protein is the green fluorescent protein (GFP) from the jellyfish, Aequorea Victoria (Chalfie M, et al., (1994) Science 263:802-805). The gene for GFP has been mutated for improved stability, spectroscopic properties, and expression in eukaryotes as well as different fluorescent colors. Examples of other fluorescent proteins include yellow fluorescent protein (YFP), blue fluorescent protein (BFP), cyan fluorescent protein (CFP), orange fluorescent protein (OFP) and red fluorescent protein (RFP). In some aspects of the invention, a reporter molecule is used to indicate differentiation of definitive endoderm to pancreatic endocrine progenitor cells. In some aspects, the reporter molecule is β-lactamase. In some aspects, the gene for reporter molecule, bla, is operably linked to a promoter of a gene that is expressed in pancreatic endocrine progenitor cells or derivatives thereof but not expressed in definitive endoderm. Derivatives of pancreatic endocrine progenitor cells include primitive beta-islet cells, beta-islet cells, alpha-islet cells, delta-islet cells, epsilon-islet cells and PP islet cells. Examples of promoters expressed in pancreatic endocrine progenitor cells but not definitive endoderm include but are not limited to an Ins1 promoter, an Ins2 promoter, a Gcg promoter, a Sst promoter, a Ppy promoter and a Ghrl1 promoter. In some aspects of the invention, the reporter molecule is BLA and the bla gene is operably linked to an Ins1 promoter. In some aspects of the invention, the bla gene is targeted to the ins1 gene in the ES genome by homologous recombination.

The preferred detection reagent for detection of the marker will depend on the identity of the marker. When the marker is an enzyme, the preferred detection reagent is a substrate, whether natural or synthetic, that is detectable after processing by the enzyme. Any type of substrate in which the processed product can be assayed should be suitable, although chromogenic and fluorogenic (e.g., substrates which become colored or fluorescent after enzyme processing) are preferred. Examples of enzyme-substrate combinations include beta-galactosidase and O-nitrophenol-b-D-pyranogalactoside (ONPG), beta-galactosidase and fluoroscein din-b-galactopyranoside (FDG) beta-galactosidase and galacton, firefly luciferase and D-luciferin, Renilla luciferase and coelenterazine, Gaussia luciferase and coelenterazine and alkaline phophotase and 5-Bromo-4-chloro-3-indolyl phosphate (BCIP). Another reporter molecule and detection reagent pair is β-lactamase and CCF2. CCF2 fluoresces green in its native state but cleavage of the β-lactam ring of CCF2; for example by β-lactamase, results in blue fluorescence.

When the reporter molecule is a fluorescent reporter, for example; GFP, YFP, RFP, etc., reporter expression can be determined by any method known in the art to detect and/or measure fluorescence. For example, cells expressing GFP may be detected by fluorescence microscopy or by fluorescence activated cell sorting analysis. In other cases, fluorescence may be measured with a fluorometer.

Reporters can be detected in live cells, fixed cells or cell extracts depending on the particular reporter construct chosen. For example, in cases were the EBs encode a fluorescent protein such as GFP, reporter expression can be analyzed from live cells by fluorescence activated cell sorting. After GFP expression has been measured, the cells can be returned to culture for future analysis. In other cases, the cells may be fixed on a tissue culture plate or microscope slide prior to detection of the reporter molecule. In other cases, the reporter protein may be secreted in the cell, for example, using a Gaussia luciferase construct. In these cases, cell supernatants are removed and analyzed for expression of the reporter molecule. In another example, cells are lysed prior to detection of the reporter molecule. This method is often used with enzymatic detection of reporter constructs, for example, chloramphenicol acetyl transferase.

Reporter molecules of the invention are operably linked to a promoter that is active in pancreatic endocrine progenitor cells or pancreatic endocrine cells but not active in primitive endoderm. Examples of pancreatic endocrine-specific promoters include, but are not limited to, an insulin 1 promoter, an insulin 2 promoter, a glucagon promoter, a somatostatin promoter, a pancreatic polypeptide promoter and a ghrelin/obestatin preprohormone promoter.

### IV. Targeting Pdx1 and Ngn3 Genes

### Targeting to the HPRT Gene

In some aspects of the invention, pdx1 and ngn3 genes are integrated into the HPRT locus. For example Ainv18 murine ES cells have been engineered to contain a reverse tet transactivator (rtTA) inserted into the ROSA26 locus and a tet-regulated promoter inserted into the 5′ region of the HPRT locus (Kyba, M. et al. 2002 Cell 109:29-37). Downstream of the tet-regulated promoter is a lox site, followed by a 5′ truncated neomycin-resistance marker. Successful recombination into the lox site of the Ainv18 cells inserts the cDNA(s) of interest downstream of the tet-regulated promoter and reconstitutes the neo^{R} ORF, allowing selection using G418. In some aspects of the invention, pdx1 and ngn3 genes are cloned into a plasmid containing a lox site. The plasmid is electroporated into Ainv18 cells and the pdx1 and ngn3 genes are integrated into the HPRT locus by means of lox-mediated recombination. In some aspects of the invention, the pdx1 and ngn3 genes are (i) under the control of an inducible promoter, (ii) linked by an IRES, and (iii) are integrated into an HPRT locus. In some aspects of the invention, a Tet-pdx1-IRES-ngn3 expression cassette is integrated into the HPRT locus.

### Targeting to the ROSA26 Locus

The design of optimal differentiation systems and appropriate readouts for screening requires genetic engineering of the ES cell, yet gene targeting reduces that gene's dosage by 50% and randomly integrated marker genes are notoriously sensitive to flanking chromatin sequences and tend to be silences during differentiation (Feng et al 2000). There is evidence that including a large (>100 kb) stretch of DNA may minimize these positional effects (Gong, S. et al. 2003 Nature 425:917-925). Many strategies use the ROSA26 locus for transgene expression due to its consistent expression in all stages of differentiation and because it does not affect differentiation or cell processes (Friedrich, G. and Soriano, P. 1991 Genes Dev. 5:1513-1523; Irion, S. et al. 2007 Nat. Biotech. 25:1477-1482; Soriano, P. 1999 Nat. Genet. 21:70-71; Strethdee, D. et al 2006 PLoS ONE 1, e4). In some aspects of the invention, a large “artificial chromosome” (BAC) of human DNA encoding Pdx1 and/or Ngn3 is integrated into the ROSA26 locus using recombination mediated cell engineering (RCME, Baer and Bode, 2001). The ROSA26 locus should not only provide a simple “landing platform” for recombination but also should allow of gene-specific expression that is not subject to positional effects and silencing. In some aspects of the invention, an artificial chromosome containing insulin promoter driving a β-lactamase reporter gene is inserted into the ROSA26 locus of ES cells or iPS cells. The resultant cells may be used to monitor the differentiation of ES cells or IPS cells into pancreas-like cells. In some aspects of the invention, the reporter molecule will be useful for research on the effects of drugs on β-islet cell growth and insulin expression. In some aspects of the invention, a pdx1 gene, an ngn3 gene and a bla gene are integrated into the ROSA26 locus. In some aspects of the invention, the pdx1 and ngn3 genes are under the control of an inducible promoter and linked by an IRES and the bla gene is under the control of a pancreatic endocrine-specific promoter and are all integrated into ROSA26 locus. In some aspects of the invention, a Tet-pdx1-IRES-ngn3 expression cassette and an ins1-bla expression cassette are integrated into the ROSA26 locus.

### V. Differentiation of ES Cells to Pancreatic Endocrine Progenitor Cells

The invention provides methods of differentiating pluripotent cells such as ES cells or iPS cells to pancreatic endocrine progenitor cells. In some aspects of the invention, pluripotent cells are first induced to differentiate into defined endoderm. Defined endoderm may then be differentiated into pancreatic progenitor cells by the overexpression of Pdx1. In some cases, pancreatic endocrine progenitor cells may be generated from defined endoderm by the simultaneous overexpression of Pdx1 and Ngn3. In other cases, pancreatic endocrine progenitor cells are derived by the sequential overexpression of Pdx1, to form pancreatic progenitor cells, followed by overexpression of Ngn3. Pancreatic endocrine progenitor cells can be further differentiated to specific pancreatic endocrine cells. For example, pancreatic endocrine progenitor cells, formed by the forced expression of Pdx1 and Ngn3 may differentiate to primitive beta-islet cells by forced expression of MafA.

Pancreatic endocrine progenitor cells of the invention may be derived from embryonic stem cells. In some aspects of the invention, the ES cells are provided by established ES cell lines. The ES cells can be derived from any species including, but not limited to, mouse, rat, hamster, rabbit, cow, pig, sheep, monkey and human. In some aspects, mouse ES cells are isolated from blastocysts by methods known (Evans et al. (1981) Nature 292:154-156; Martin, GR (1981) Proc. Natl. Acad. Sci. USA 78:7634-7638). In some aspects of the invention, human ES cells are isolated from blastocysts (see for example, U.S. Pat. No. 5,843,780; U.S. Pat. No. 6,200,806; Thomson et al., Proc. Natl. Acad. Sci. USA 92:7844, 1995). In some aspects, in vitro fertilized (IVF) embryos or one-cell human embryos can be expanded to the blastocyst stage (Bongso et al., Hum Reprod 4: 706, 1989).

Assays known in the art may be performed to confirm the undifferentiated state of ES cells. For example, antibodies to OCT3/4, Nanog, SSEA-−4, TRA-1-60 and TRA-1-81 may be used to characterize cells. Cells that stain positive for these ES markers are indicative of an undifferentiated state. ES cell lines can be assessed for pluripotency and their ability to differentiate into all three germ layers using antibodies directed against marker proteins. For example; ectoderm markers include but are not limited to SOX1, Nestin and β-III-Tubulin; mesoderm markers include but are not limited to Brachyury and α-pan-Mysosin; and endoderm markers include but are not limited to FOXA2 and AFP.

In some aspects of the invention, pancreatic endocrine progenitor cells are derived from ES cells that have been differentiated into definitive endoderm. Definitive endoderm can be derived from ES by methods known in the art; for example, U.S. Patent Appl. Pub. Nos. 2006/0276420 and 2006/0003446 and U.S. Pat. Nos. 7,033,831 and 7,326,572. In some aspects of the invention, cell populations enriched for endoderm may be obtained by culturing embryonic stem cells in the absence of serum and in the presence of the growth factor activin and isolating cells that express brachyury. The amount of activin is sufficient to induce differentiation of embryonic stem cells to endoderm. Such differentiation may be measured by assaying for the expression of genes associated with endoderm development, including for example HNF3β, Mixl-1, Sox17, Hex-1 or Pdx1. In some cases, the concentration of activin is at least about 30 ng/ml. In some cases the concentration of activin is about 100 ng/ml. In some cases, cells are cultured in the presence of activin for about two to about ten days.

In some cases, the definitive endoderm is derived from human ES cells. Definitive endoderm may be identified by expression of known markers of definitive endoderm. Markers of human definitive endoderm include, but are not limited to, CXCR4, Sox17, GSC, Fox-A2 and c-Kit. In some cases, the definitive endoderm is derived from mouse ES cells. Markers of mouse definitive endoderm include, but are not limited to Sox17, Fox-A2, GSC, claudin-6 and Hex-1. After definitive endoderm has been derived from ES cells, pancreatic endocrine progenitor cells can be derived from definitive endoderm by forced expression of Pdx1 and Ngn3. In some aspects of the invention, Pdx1 and Ngn3 are expressed following integration of pdx1 and ngn3 genes in the ES genome. In other cases, Pdx1 and Ngn3 are expressed following transient introduction of pdx1 and ngn3 genes. Pancreatic endocrine progenitor cells may be identified; for example, by the detection of expression of insulin mRNA.

In some cases, Ngn3 is expressed at the same time as Pdx1. Differentiation toward pancreatic endocrine progenitor cells may be determined by measuring insulin mRNA expression. Insulin mRNA expression is not detected in definitive endoderm but is expressed in pancreatic endocrine progenitor cells.

In other cases, Pdx1 is expressed first to generate pancreatic progenitor cells. The resultant population of pancreatic progenitor cells is then analyzed for the expression of insulin. If insulin mRNA expression is detected in the population of pancreatic progenitor cells, Ngn3 may then be expressed to generate pancreatic endocrine progenitor cells. An increase in the expression of insulin indicates further differentiation from definitive endoderm toward pancreatic endocrine progenitor cells. In some cases, expression of insulin mRNA in the population of pancreatic endocrine progenitor cells is increased two-fold over the level of insulin mRNA expression in the population of pancreatic progenitor cells generated by forced expression of Pdx1. In other cases expression of insulin mRNA is increased ten-fold over the level of insulin mRNA expression in population of pancreatic progenitor cells. In other cases expression of insulin mRNA is increased 100-fold over the level of insulin mRNA expression in population of pancreatic progenitor cells.

An illustrative but non-limiting example of a method to generate pancreatic endocrine progenitor cell from ES cells by overexpression of Pdx1 and Ngn3 is as follows. Mouse ES cells are maintained on MEF feeder cells. Cells are then passaged onto plates without MEF feeder cells for about one day. On day 0, ES cells are induced to form embryoid bodies (EBs). On about day 2, EBs are incubated in the presence of activin A to form endoderm. In cases where the pdx1 and ngn3 genes are delivered transiently, a vector for the expression of Pdx1 and Ngn3; for example, Tet-pdx1-IRES-ngn3, is introduced into the EBs on about days 4-6. In cases where expression of Pdx1 and Ngn3 is under the control of an inducible promoter, the EBs are incubated with the activator of the promoter, such as doxycycline in the case of Tet-pdx1-IRES-ngn3, on about day 6. In some aspects of the invention, a vector encoding a reporter molecule such as Ins1-BLA is also introduced to the EBs on about day 6. In some cases, on about day 9, cells are harvested for analysis. In some cases, pancreatic endocrine progenitor cells are maintained as a monolayer. Cells can be analyzed for pancreatic endocrine progenitor cell characteristics by a number of methods known in the art including, but not limited to RT-PCR, immunohistochemistry and enzyme assays. In cases where Ins1-BLA is introduced into the EBs, cells can be assayed for development of pancreatic endocrine progenitor characteristics by BLA assay.

Another illustrative, but non-limiting, example of a method to generate pancreatic endocrine progenitor cell from ES cells in which Pdx1 and Ngn3 have been stably introduced; for example, Tet-pdx1-IRES-ngn3 Ainv cells, is as follows. Undifferentiated ES cells are maintained on MEF feeder cells. On about day −4, cells are plated on gelatinized culture dishes in the absence of MEF feeder cells. On about day −2 cells are passaged in a pre-differentiation step. On day 0, EBs are induced by culture in SFD complete medium. On about day 2, EBs are dissociated and replated in the presence of activin A. On about day 4, EBs are reaggregated and Pdx1 and Ngn3 expression is induced; for example, by addition of Dox to the media. On about day 6, cells are expanded on low attachment plates. Induction of expression of Pdx1 and Ngn3 is continued. On about days 9, 11 and 13 cells are fed and induction of expression of Pdx1 and Ngn3 is continued. On about day 16, cells are harvested and analyzed. Cells can be analyzed for pancreatic endocrine progenitor cell characteristics by a number of methods known in the art including, but not limited to RT-PCR, immunohistochemistry and enzyme assays. In some cases, Ins1-BLA is also stably introduced into to the ES cells. In these cases, cells can be assayed for development of pancreatic endocrine progenitor characteristics by BLA assay.

Another illustrative, but non-limiting, example of a method to generate pancreatic endocrine progenitor cell from ES cells in which Pdx1 and Ngn3 have been stably introduced; for example, Tet-pdx1-IRES-ngn3 Ainv cells, is as follows. Undifferentiated ES cells are maintained on MEF feeder cells. On about day −4, cells are plated on gelatinized culture dishes in the absence of MEF feeder cells. On about day −2 cells are passaged in a pre-differentiation step. On day 0, ES cells are plated as a monolayer in SFD complete medium. On about day 2, cells are dissociated and replated in the presence of activin A. On about day 4, cells are dissociated and Pdx1 and Ngn3 expression is induced; for example, by addition of Dox to the media. On about day 6, cells are expanded. Induction of expression of Pdx1 and Ngn3 is continued. On about days 9, 11 and 13 cells are fed and induction of expression of Pdx1 and Ngn3 is continued. In some cases, cells are harvested and analyzed on about day 16. Cells can be analyzed for pancreatic endocrine progenitor cell characteristics by a number of methods known in the art including, but not limited to RT-PCR, immunohistochemistry and enzyme assays. In some cases, Ins1-BLA is also stably introduced into to the ES cells. In these cases, cells can be assayed for development of pancreatic endocrine progenitor characteristics by BLA assay. In other cases, pancreatic endocrine progenitor cells are maintained as a monolayer.

Following the induction of pancreatic endocrine progenitor cells from ES cells by overexpression of Pdx1 and Ngn3, pancreatic endocrine progenitor cells are induced to a monolayer formation. In some cases, this allows cells to make a maturation step to make glucose response adult phenotype.

In some aspects of the invention, ES cells are modified to overexpress their endogenous Pdx1 and Ngn3 genes. In some cases, Pdx1 and Ngn3 expression is induced by one or more agents; for example but not limited to, a small molecule inducer, a regulatory RNA molecule and the like. In some cases, Pdx1 and Ngn3 expression is enhanced in a cell population by inactivating inhibitors of Pdx1 and Ngn3. Agents that induce or enhance expression of Pdx1 and/or Ngn3 can be identified by contacting said agents with ES cells and measuring expression of Pdx1 and/or Ngn3. In some aspects of the invention, the temporal effects of the agent on Pdx1 and Ngn3 expression can be determined by a time-course analysis in which ES cells are contacted with the agent, sampled at varying times and measured for Pdx1 and Ngn3 expression. Agents identified by such a screening process can then be used to induce ES cells to form pancreatic endocrine progenitor cells.

In some aspects of the invention, ES cells that express endogenous Pdx1 and/or Ngn3 are selected from a population of ES cells. Cells that express Pdx1 and/or Ngn3 can be isolated by a number of methods. For example, genes expressing reporter molecules or selectable markers can be linked to expression of Pdx1 and/or Ngn3. In some cases, a reporter protein or selectable marker is included in fusion proteins with Pdx1 and/or Ngn3. In some cases, a reporter molecule or selectable marker operably linked to a pdx1 and/or ngn3 promoter is introduced into the ES cells. Methods of selecting cells based on reporter molecules and/or selectable markers are known in the art and include, but are not limited to FACs and drug resistance. Isolated cells expressing Pdx1 and Ngn3 can be used to generate pancreatic endocrine progenitor cells and their progeny.

The invention provides methods to produce pancreatic endocrine progenitor cells or primitive beta-islet cells from definitive endoderm by forced expression of Pdx1, Ngn3 and MafA. In some aspects of the invention, Pdx1, Ngn3 and MafA are expressed following integration of pdx1, ngn3 and mafA genes in the ES genome. In some aspects of the invention, Pdx1, Ngn3 are expressed following integration of pdx1 and ngn3 genes in the ES genome and MafA is expressed following transient introduction of the mafA gene. In other cases, Pdx1, Ngn3 and MafA are expressed following transient introduction of pdx1, ngn3 and mafA genes.

In some aspects of the invention, definitive endoderm is derived from ES cells as described above. In some cases, definitive endoderm is derived from human ES cells. In some cases, definitive endoderm is derived from mouse ES cells. Definitive endoderm may be identified using known markers of definitive endoderm as described above. Differentiation toward pancreatic endocrine progenitor cells may be induced by the simultaneous or sequential expression of Pdx1 and Ngn3 as discussed above. In some aspects of the invention, expression of MafA is initiated at the same time as expression of Pdx1 and Ngn3. In some cases, pancreatic endocrine progenitor cells are induced by expression of Pdx1 and Ngn3 and cells are analyzed for expression of insulin mRNA. The expression of insulin; for example, insulin mRNA, indicates differentiation from definitive endoderm toward pancreatic endocrine progenitor cells. If insulin expression is detected, expression of MafA may then be induced to differentiate the cells further toward primitive beta-islet cells.

An illustrative but non-limiting example of a method to generate pancreatic endocrine progenitor cells and/or primitive beta-islet cells from ES cells by overexpression of Pdx1, Ngn3 and MafA is as follows. Mouse ES cells are maintained on MEF feeder cells. Cells are then passaged onto plates without MEF feeder cells for about one day. On day 0, ES cells are induced to form embryoid bodies (EBs). On about day 2, EBs are incubated in the presence of activin A to form endoderm. In cases where the pdx1, ngn3 and mafA genes are delivered transiently, a vector for the expression of Pdx1 and Ngn3; for example, Tet-pdx1-IRES-ngn3, and a vector for the expression of MafA; for example, pCMV-mafA, are introduced into the EBs on about days 4-6. In cases where expression of Pdx1, Ngn3 and MafA is under the control of inducible promoters, the EBs are incubated with the activators of the promoters, such as doxycycline in the case of Tet-pdx1-IRES-ngn3, on about day 6. In some aspects of the invention, a vector encoding a reporter molecule such as Ins1-BLA is also introduced to the EBs on about day 6. In some cases, on about day 9, cells are harvested for analysis. In some cases, pancreatic endocrine progenitor cells are maintained as a monolayer. Cells can be analyzed for pancreatic endocrine progenitor cell characteristics by a number of methods known in the art including, but not limited to RT-PCR, immunohistochemistry and enzyme assays. In cases where Ins1-BLA is introduced into the EBs, cells can be assayed for development of pancreatic endocrine progenitor characteristics by BLA assay.

Another illustrative, but non-limiting, example of a method to generate pancreatic endocrine progenitor cell and/or primitive beta-islet cells from ES cells in which Pdx1 and Ngn3 have been stably introduced and MafA is introduced transiently to the cells is as follows. Undifferentiated ES cells, for example, Tet-pdx1-IRES-ngn3 Ainv cells, are maintained on MEF feeder cells. On about day −4, cells are plated on gelatinized culture dishes in the absence of MEF feeder cells. On about day −2 cells are passaged in a pre-differentiation step. On day 0, EBs are induced by culture in SFD complete medium. On about day 2, EBs are dissociated and replated in the presence of activin A. On about day 4, EBs are reaggregated and Pdx1 and Ngn3 expression is induced; for example, by addition of Dox to the media. On about day 6, a vector for the expression of MafA is introduced into the cells and suspension culture is continued in low attachment plates. Induction of expression of Pdx1 and Ngn3 is continued. On about days 9, 11 and 13 cells are fed and induction of expression of Pdx1 and Ngn3 is continued in addition to the constitutive expression of MafA. On about day 16, cells are harvested and analyzed. Cells can be analyzed for pancreatic endocrine progenitor cell characteristics by a number of methods known in the art including, but not limited to RT-PCR, immunohistochemistry and enzyme assays. In some cases, Ins1-BLA is also stably introduced into to the ES cells. In these cases, cells can be assayed for development of pancreatic endocrine progenitor characteristics by BLA assay.

Another illustrative, but non-limiting, example of a method to generate pancreatic endocrine progenitor cell from ES cells in which Pdx1 and Ngn3 have been stably introduced and MafA is introduced transiently to the cells is as follows. Undifferentiated ES cells, for example, Tet-pdx1-IRES-ngn3 Ainv cells, are maintained on MEF feeder cells. On about day −4, cells are plated on gelatinized culture dishes in the absence of MEF feeder cells. On about day-2 cells are passaged in a pre-differentiation step. On day 0, ES cells are plated as a monolayer in SFD complete medium. On about day 2, cells are dissociated and replated in the presence of activin A. On about day 4, cells are dissociated and Pdx1 and Ngn3 expression is induced; for example, by addition of Dox to the media. On about day 6, cells are dissociated and a vector for the expression of MafA is introduced to the cells. Induction of expression of Pdx1 and Ngn3 is continued. On about days 9, 11 and 13 cells are fed and induction of expression of Pdx1 and Ngn3 is continued in addition to the constitutive expression of MafA. In some cases, cells are harvested and analyzed on about day 16. Cells can be analyzed for pancreatic endocrine progenitor cell characteristics by a number of methods known in the art including, but not limited to RT-PCR, immunohistochemistry and enzyme assays. In some cases, Ins1-BLA is also stably introduced into to the ES cells. In these cases, cells can be assayed for development of pancreatic endocrine progenitor characteristics by BLA assay. In other cases, pancreatic endocrine progenitor cells are maintained as a monolayer.

### VI. Differentiation of iPS Cells to Pancreatic Endocrine Progenitor Cells

Pancreatic endocrine progenitor cells of the invention may be derived from iPS cells. In some aspects of the invention, the iPS cells are provided by established iPS cell lines. The iPS cells can be derived from any species including, but not limited to, mouse, rat, hamster, rabbit, cow, pig, sheep, monkey and human. iPS cells may be derived by methods known in the art including the use integrating viral vectors to deliver the genes that promote reprogramming (Takahashi, K. and Yamanaka, S., 2006 Cell 126:663-676; Okita, K. et al., 2007 Nature 448:313-317; Nakagawa, M. et al., 2007 Nat. Biotechnol. 26:101-106; Takahashi, K. et al., 2007 Cell 131:1-12; Meissner A. et al. 2007 Nat. Biotech. 25:1177-1181; Yu, J. et al. 2007 Science 318:1917-1920; Park, I. H. et al. 2008 Nature 451:141-146; Stadtfeld, M. et al. 2008 Sciencexpress, and U.S. Pat. Application Publication No. 2008/0233610. An example of differentiation of iPSC induction using repeated plasmid transfection is provided by Okita, K. et al., (2008) Sciencexpress. An example of differentiation of iPSC into insulin-secreting islet like cells is provided by Tateishi, K. et al., (2008) J. Biol. Chem.

Assays known in the art may be performed to confirm the undifferentiated state of iPS cells. For example, antibodies to OCT3/4, Nanog, SSEA-4, TRA-1-60 and TRA-1-81 may be used to characterize cells. Cells that stain positive for these ES markers are indicative of an undifferentiated state. iPS cell lines can be assessed for pluripotency and their ability to differentiate into all three germ layers using antibodies directed against marker proteins. For example; ectoderm markers include but are not limited to SOX1, Nestin and β-III-Tubulin; mesoderm markers include but are not limited to Brachyury and α-pan-Mysosin; and endoderm markers include but are not limited to FOXA2 and AFP.

Cell populations enriched for endoderm may be obtained by culturing iPSC in the absence of serum and in the presence of the growth factor activin. The amount of activin is sufficient to induce differentiation of iPSC to endoderm. In some cases, cells that express brachyury are isolated following growth in the presence of activin. In some cases, cells are grown in the presence of activin for about two to about ten days. Differentiation of iPS to definitive endoderm may be measured by assaying for the expression of genes associated with endoderm development, including for example HNF3β, mixl-1, sox17 or hex. In some aspects of the invention, the concentration of activin is at least about 30 ng/ml. In another aspect of the invention, the concentration of activin is about 100 ng/ml.

In some cases, the definitive endoderm is derived from human iPS cells. Definitive endoderm may be identified by expression of known markers of definitive endoderm. Markers of human definitive endoderm include, but are not limited to, CXCR4, Sox17, GSC, Fox-A2 and c-Kit. In some cases, the definitive endoderm is derived from mouse iPS cells. Markers of mouse definitive endoderm include, but are not limited to Sox17, Fox-A2, GSC, claudin-6 and Hex-1. After definitive endoderm has been derived from iPS cells, pancreatic endocrine progenitor cells can be derived from definitive endoderm by forced expression of Pdx1 and Ngn3 as described for pancreatic endocrine progenitor cells derived from endoderm derived from ES cells. In some aspects of the invention, Pdx1 and Ngn3 are expressed following integration of pdx1 and ngn3 genes in the iPS genome. In other cases, Pdx1 and Ngn3 are expressed following transient introduction of pdx1 and ngn3 genes. Pancreatic endocrine progenitor cells may be identified; for example, by the detection of expression of insulin mRNA.

In some cases, Ngn3 is expressed at the same time as Pdx1. Differentiation toward pancreatic endocrine progenitor cells may be determined by measuring insulin mRNA expression. Insulin mRNA expression is not detected in definitive endoderm but is expressed in pancreatic endocrine progenitor cells.

In other cases, Pdx1 is expressed first to generate pancreatic progenitor cells. The resultant population of pancreatic progenitor cells is then analyzed for the expression of insulin. If insulin mRNA expression is detected in the population of pancreatic progenitor cells, Ngn3 may then be expressed to generate pancreatic endocrine progenitor cells. An increase in the expression of insulin indicates further differentiation from definitive endoderm toward pancreatic endocrine progenitor cells. In some cases, expression of insulin mRNA in the population of pancreatic endocrine progenitor cells is increased two-fold over the level of insulin mRNA expression in the population of pancreatic progenitor cells generated by forced expression of Pdx1. In other cases expression of insulin mRNA is increased ten-fold over the level of insulin mRNA expression in population of pancreatic progenitor cells. In other cases expression of insulin mRNA is increased 100-fold over the level of insulin mRNA expression in population of pancreatic progenitor cells.

An illustrative but non-limiting example of a method to generate pancreatic endocrine progenitor cell from iPS cells by overexpression of Pdx1 and Ngn3 is as follows. iPS cells are maintained on MEF feeder cells. Cells are then passaged onto plates without MEF feeder cells for about one day. On day 0, iPS cells are induced to form embryoid bodies (EBs). On about day 2, EBs are incubated in the presence of activin A to form endoderm. In cases where the pdx1 and ngn3 genes are delivered transiently, a vector for the expression of Pdx1 and Ngn3; for example, Tet-pdx1-IRES-ngn3, is introduced into the EBs on about days 4-6. In cases where expression of Pdx1 and Ngn3 is under the control of an inducible promoter, the EBs are incubated with the activator of the promoter, such as doxycycline in the case of Tet-pdx1-IRES-ngn3, on about day 6. In some aspects of the invention, a vector encoding a reporter molecule such as Ins1-BLA is also introduced to the EBs on about day 6. In some cases, on about day 9, cells are harvested for analysis. In some cases, pancreatic endocrine progenitor cells are maintained as a monolayer. Cells can be analyzed for pancreatic endocrine progenitor cell characteristics by a number of methods known in the art including, but not limited to RT-PCR, immunohistochemistry and enzyme assays. In cases where Ins1-BLA is introduced into the EBs, cells can be assayed for development of pancreatic endocrine progenitor characteristics by BLA assay. In some cases, a vector encoding a reporter molecule is introduced at any time during the differentiation process; for example but not limited to about days 0, 1, 2, 3, 4, 5, 6, 7, 8, 9, or 10. In some cases, a vector encoding a reporter molecule in introduced into the cells before identification of pancreatic endocrine progenitor cells. In some cases, a vector encoding a reporter molecule in introduced into the cells before identification of pancreatic endocrine progenitor cells for sufficient time to allow expression of the reporter molecule to assist in the identification of pancreatic endocrine progenitor cells or their derivatives; for example, three days before the identification of pancreatic endocrine progenitor cells or their derivatives.

Another illustrative, but non-limiting, example of a method to generate pancreatic endocrine progenitor cell from iPS cells in which Pdx1 and Ngn3 have been stably introduced is as follows. Undifferentiated iPS cells are maintained on MEF feeder cells. On about day −4, cells are plated on gelatinized culture dishes in the absence of MEF feeder cells to remove feeder cells and as a pre-differentiation step. On about day −2 the cells are passaged again. On day 0, cells are induced to form EBs by culturing them on low attachment plates in SFD complete medium. On about day 2, EBs are dissociated and replated in the presence of activin A. On about day 4, EBs are reaggregated and Pdx1 and Ngn3 expression is induced; for example, by addition of Dox to the media. On about day 6, cells are expanded on low attachment plates. Induction of expression of Pdx1 and Ngn3 is continued. On about days 9, 11 and 13 cells are fed and induction of expression of Pdx1 and Ngn3 is continued. On about day 16, cells are harvested and analyzed. Cells can be analyzed for pancreatic endocrine progenitor cell characteristics by a number of methods known in the art including, but not limited to RT-PCR, immunohistochemistry and enzyme assays. In some cases, Ins1-BLA is also stably introduced into to the iPS cells prior to differentiation by targeting BLA to the endogenous insulin gene. In these cases, cells can be assayed for development of pancreatic endocrine progenitor characteristics by BLA assay.

Another illustrative, but non-limiting, example of a method to generate pancreatic endocrine progenitor cell from iPS cells in which Pdx1 and Ngn3 have been stably introduced, is as follows. Undifferentiated iPS cells are maintained on MEF feeder cells. On about day −4, cells are plated on gelatinized culture dishes in the absence of MEF feeder cells to remove the MEF feeders and as a pre-differentiation step. On about day −2 the cells are passaged again. On day 0, iPS cells are plated as a monolayer in SFD complete medium. On about day 2, cells are dissociated and replated in the presence of activin A. On about day 4, cells are dissociated and Pdx1 and Ngn3 expression is induced; for example, by addition of Dox to the media. On about day 6, cells are expanded. Induction of expression of Pdx1 and Ngn3 is continued. On about days 9, 11 and 13 cells are fed and induction of expression of Pdx1 and Ngn3 is continued. In some cases, cells are harvested and analyzed on about day 16. Cells can be analyzed for pancreatic endocrine progenitor cell characteristics by a number of methods known in the art including, but not limited to RT-PCR, immunohistochemistry and enzyme assays. In some cases, Ins 1-BLA is also stably introduced into to the iPS cells. In these cases, cells can be assayed for development of pancreatic endocrine progenitor characteristics by BLA assay. In other cases, pancreatic endocrine progenitor cells are maintained as a monolayer.

Following the induction of pancreatic endocrine progenitor cells from iPS cells by overexpression of Pdx1 and Ngn3, pancreatic endocrine progenitor cells are induced to a monolayer formation. In some cases, this allows cells to make a maturation step to make glucose response adult phenotype.

In some aspects of the invention, iPS cells are modified to overexpress their endogenous Pdx1 and Ngn3 genes. In some cases, Pdx1 and Ngn3 expression is induced by one or more agents; for example but not limited to, a small molecule inducer, a regulatory RNA molecule and the like. In some cases, Pdx1 and Ngn3 expression is enhanced in a cell population by inactivating inhibitors of Pdx1 and Ngn3. Agents that induce or enhance expression of Pdx1 and/or Ngn3 can be identified by contacting said agents with iPS cells and measuring expression of Pdx1 and/or Ngn3. In some aspects of the invention, the temporal effects of the agent on Pdx1 and Ngn3 expression can be determined by a time-course analysis in which iPS cells are contacted with the agent, sampled at varying times and measured for Pdx1 and Ngn3 expression. Agents identified by such a screening process can then be used to induce iPS cells to form pancreatic endocrine progenitor cells.

In some aspects of the invention, iPS cells that express endogenous Pdx1 and/or Ngn3 are selected from a population of iPS cells. Cells that express Pdx1 and/or Ngn3 can be isolated by a number of methods. For example, genes expressing reporter molecules or selectable markers can be linked to expression of Pdx1 and/or Ngn3. In some cases, a reporter protein or selectable marker in included in a fusion proteins with Pdx1 and/or Ngn3. In some cases, a reporter molecule or selectable marker operably linked to a pdx1 and/or ngn3 promoter is introduced into the iPS cells. Methods of selecting cells based on reporter molecules and/or selectable markers are known in the art and include, but are not limited to FACs and drug resistance. Isolated cells expressing Pdx1 and Ngn3 can be used to generate pancreatic endocrine progenitor cells and their progeny.

The invention provides methods to produce pancreatic endocrine progenitor cells and/or primitive beta-islet cells from iPS derived definitive endoderm by forced expression of Pdx1, Ngn3 and MafA. In some aspects of the invention, Pdx1, Ngn3 and MafA are expressed following integration of pdx1, ngn3 and mafA genes in the iPS genome. In some aspects of the invention, Pdx1, Ngn3 are expressed following integration of pdx1 and ngn3 genes in the iPS genome and MafA is expressed following transient introduction of the mafA gene. In other cases, Pdx1, Ngn3 and MafA are expressed following transient introduction of pdx1, ngn3 and mafA genes.

In some aspects of the invention, definitive endoderm is derived from iPS cells as described above. In some cases, definitive endoderm is derived from human iPS cells. In some cases, definitive endoderm is derived from mouse iPS cells. Definitive endoderm may be identified using known markers of definitive endoderm as discussed above. Differentiation toward pancreatic endocrine progenitor cells may be induced by the simultaneous or sequential expression of Pdx1 and Ngn3 as described above. In some aspects of the invention, expression of MafA is initiated at the same time as expression of Pdx1 and Ngn3. In some cases, pancreatic endocrine progenitor cells are induced by expression of Pdx1 and Ngn3 and cells are analyzed for expression of insulin. An increase in the expression of insulin indicates further differentiation from definitive endoderm to pancreatic endocrine progenitor cells. If insulin expression is detected, expression of MafA may then be initiated to differentiate the cells further toward primitive beta.

An illustrative but non-limiting example of a method to generate pancreatic endocrine progenitor cells and/or primitive beta-islet cells from iPS cells by overexpression of Pdx1, Ngn3 and MafA is as follows. iPS cells are maintained on MEF feeder cells. Cells are then passaged onto plates without MEF feeder cells for about one day. On day 0, iPS cells are induced to form embryoid bodies (EBs). On about day 2, EBs are incubated in the presence of activin A to form endoderm. In cases where the pdx1, ngn3 and mafA genes are delivered transiently, a vector for the expression of Pdx1 and Ngn3; for example, Tet-pdx1-IRES-ngn3, and a vector for the expression of MafA; for example, pCMV-mafA, are introduced into the EBs on about days 4-6. In cases where expression of Pdx1, Ngn3 and MafA is under the control of inducible promoters, the EBs are incubated with the activators of the promoters, such as doxycycline in the case of Tet-pdx1-IRES-ngn3, on about day 6. In some aspects of the invention, a vector encoding a reporter molecule such as Ins1-BLA is also introduced to the EBs on about day 6. In some cases, on about day 9, cells are harvested for analysis. In some cases, pancreatic endocrine progenitor cells are maintained as a monolayer. Cells can be analyzed for pancreatic endocrine progenitor cell characteristics by a number of methods known in the art including, but not limited to RT-PCR, immunohistochemistry and enzyme assays. In cases where Ins1-BLA is introduced into the EBs, cells can be assayed for development of pancreatic endocrine progenitor characteristics by BLA assay.

Another illustrative, but non-limiting, example of a method to generate pancreatic endocrine progenitor cell and/or primitive beta-islet cells from iPS cells in which Pdx1 and Ngn3 have been stably introduced and MafA is introduced transiently to the cells is as follows. Undifferentiated iPS cells are maintained on MEF feeder cells. On about day −4, cells are plated on gelatinized culture dishes in the absence of MEF feeder cells to remove feeders and as a predifferentiation step. On about day −2 cells are passaged again. On day 0, cells are induced to form EBs by culturing them on low attachment plates in SFD complete medium. On about day 2, EBs are dissociated and replated in the presence of activin A. On about day 4, EBs are reaggregated and Pdx1 and Ngn3 expression is induced; for example, by addition of Dox to the media. On about day 6, cells are expanded on low attachment plates and a vector for the expression of MafA is introduced into the cells and suspension culture is continued in low attachment plates. Induction of expression of Pdx1 and Ngn3 is continued. On about days 9, 11 and 13 cells are fed and induction of expression of Pdx1 and Ngn3 is continued in addition to the constitutive expression of MafA. On about day 16, cells are harvested and analyzed. Cells can be analyzed for pancreatic endocrine progenitor cell characteristics by a number of methods known in the art including, but not limited to RT-PCR, immunohistochemistry and enzyme assays. In some cases, Ins1-BLA is also stably introduced into to the iPS cells. In these cases, cells can be assayed for development of pancreatic endocrine progenitor characteristics by BLA assay.

Another illustrative, but non-limiting, example of a method to generate pancreatic endocrine progenitor cells and/or primitive beta-islet cells from iPS cells in which Pdx1 and Ngn3 have been stably introduced and MafA is introduced transiently to the cells is as follows. Undifferentiated iPS cells are maintained on MEF feeder cells. On about day −4, cells are plated on gelatinized culture dishes in the absence of MEF feeder cells to remove feeders and as a pre-differentiation step. On about day −2 cells are passaged again. On day 0, iPS cells are plated as a monolayer in SFD complete medium. On about day 2, cells are dissociated and replated in the presence of activin A. On about day 4, cells are dissociated and Pdx1 and Ngn3 expression is induced; for example, by addition of Dox to the media. On about day 6, cells are expanded and a vector for the expression of MafA is introduced into the cells and suspension culture is continued in low attachment plates. Induction of expression of Pdx1 and Ngn3 is continued. On about days 9, 11 and 13 cells are fed and induction of expression of Pdx1 and Ngn3 is continued in addition to the constitutive expression of MafA. In some cases, cells are harvested and analyzed on about day 16. Cells can be analyzed for pancreatic endocrine progenitor cell characteristics by a number of methods known in the art including, but not limited to RT-PCR, immunohistochemistry and enzyme assays. In some cases, Ins1-BLA is also stably introduced into to the iPS cells prior to differentiation by targeting BLA to the endogenous insulin gene. In these cases, cells can be assayed for development of pancreatic endocrine progenitor characteristics by BLA assay. In other cases, pancreatic endocrine progenitor cells are maintained as a monolayer.

### VII. Methods to Produce ES Cells Modified to Overexpress Pdx1 and Ngn3

The invention provides methods to produce ES cells that are modified to overexpress Pdx1 and Ngn3. In some aspects of the invention, ES cells are modified to overexpress Pdx1 and Ngn3 by transiently introducing pdx1 and ngn3 genes. The introduction of the pdx1 and ngn3 genes can be by methods known in the art. In some aspects of the invention, a mafA gene is also introduced to the ES cells. In some aspects of the invention, expression of pdx1, ngn3 and/or mafA is initiated by transiently introducing the genes to the cells.

In some aspects of the invention, ES cells are modified to overexpress Pdx1 and Ngn3 by stably introducing pdx1 and ngn3 genes under the control of an inducible promoter into the ES cells. In some aspects, ES cells are modified to overexpress Pdx1 and Ngn3 by integrating pdx1 and ngn3 genes, under the control of one or more inducible promoters, into the ES genome. In some cases, the pdx1 and ngn3 genes are on separate expression cassettes and in some cases, the pdx1 and ngn3 genes are on the same expression cassette. For example, in some cases the pdx1 and ngn3 genes are under the control of an inducible promoter and are linked by an internal ribosome entry site. In some aspects of the invention, the pdx1 and ngn3 genes are targeted to one or more specific sites in the ES genome; for example, the pdx1 and ngn3 genes can be targeted to the HPRT locus. In some aspects of the invention, targeting the pdx1 and ngn3 genes is achieved using a recombinase system; for example, a cre-lox recombinase system. In some aspects, the invention provides a method of producing ES cells modified to overexpress Pdx1 and Ngn3 by stably integrating an expression cassette encoding the pdx1 and ngn3 genes under the control of an inducible promoter and linked by an IRES. In some cases, the inducible promoter is a tetracycline inducible promoter. In some cases the pdx1 and ngn3 genes are targeted to the HPRT gene of Ainv18 ES cells by cre-lox recombination. In some aspects, the invention provides methods to produce ES cells modified to overexpress MafA in addition to Pdx1 and Ngn3. The mafA gene may be stably integrated in the ES cell genome or may be delivered transiently.

In some aspects of the invention, a reporter molecule is also stably introduced into the ES cells. In some cases, the reporter molecule in under the control of a promoter expressed in pancreatic endocrine progenitor cells or derivatives thereof but not expressed in primitive endoderm. In some cases the promoter is an ins1 promoter and the reporter molecule is a bla gene. In some cases, the reporter expression construct is stably integrated into the ES genome. In some cases, the reporter expression construct is integrated into the ins1 locus. In some cases, the reporter expression construct is targeted by homologous recombination. In some cases the reporter expression construct is targeted by using a recombinase system; for example, a cre-lox recombination system. In some cases, the reporter expression construct is introduced into ES cells before the pdx1 and ngn3 genes are introduced into the ES cells. In some cases reporter expression construct is introduced into ES cells after the pdx1 and ngn3 genes are introduced into the ES cells. In some cases, the reporter expression construct is introduced into ES cells at the same time as the pdx1 and ngn3 genes are introduced into the ES cells.

Once an ES cell is modified to overexpress Pdx1 and Ngn3, the stable integration of the pdx1 and ngn3 genes can be verified by methods known in the art. For example, PCR can be used to check proper integration of the pdx1 and ngn3 genes into a targeted integration site. Expression of the pdx1 and ngn3 genes following induction can be detected by RT-PCR. Immunohistochemistry can also be used to show expression of Pdx1 and Ngn3 in cells following induction. Likewise, stable integration of mafA gene can be verified by methods known in the art.

### VIII. Methods to Produce iPS Cells Modified to Overexpress Pdx1 and Ngn3

The invention provides methods to produce iPS cells that are modified to overexpress Pdx1 and Ngn3 and optionally MafA. In some aspects of the invention, iPS cells are modified to overexpress Pdx1 and Ngn3 by transiently introducing pdx1 and ngn3 genes. In some cases, genes encoding Pdx1 and Ngn3 are introduced to differentiated cells prior to reprogramming to iPS cells. In some cases, genes encoding Pdx1 and Ngn3 are introduced to iPS cells after reprogramming. In some cases, genes encoding Pdx1 and Ngn3 are introduced to cells during the reprogramming process. The introduction of the pdx1 and ngn3 genes can be by methods known in the art. In some aspects of the invention, a mafA gene is also introduced to the iPS cells. In some aspects of the invention, expression of pdx1, ngn3 and/or mafA is initiated by transiently introducing the genes to the cells.

In some aspects of the invention, iPS cells are modified to overexpress Pdx1 and Ngn3 by stably introducing pdx1 and ngn3 genes under the control of an inducible promoter into the iPS cells. In some cases, genes encoding Pdx1 and Ngn3 are introduced to differentiated cells prior to reprogramming to iPS cells. In some cases, genes encoding Pdx1 and Ngn3 are introduced to iPS cells after reprogramming. In some cases, genes encoding Pdx1 and Ngn3 are introduced to cells during the reprogramming process. In some aspects, iPS cells are modified to overexpress Pdx1 and Ngn3 by integrating pdx1 and ngn3 genes, under the control of one or more inducible promoters, into the iPS genome. In some cases, the pdx1 and ngn3 genes are on separate expression cassettes and in some cases, the pdx1 and ngn3 genes are on the same expression cassette. For example, in some cases the pdx1 and ngn3 genes are under the control of an inducible promoter and are linked by an internal ribosome entry site. In some aspects of the invention, the pdx1 and ngn3 genes are targeted to one or more specific sites in the iPS genome; for example, the pdx1 and ngn3 genes can be targeted to the HPRT locus. In some aspects of the invention, targeting the pdx1 and ngn3 genes is achieved using a recombinase system; for example, a cre-lox recombinase system. In some aspects, the invention provides a method of producing iPS cells modified to overexpress Pdx1 and Ngn3 by stably integrating an expression cassette encoding the pdx1 and ngn3 genes under the control of an inducible promoter and linked by an IRES. In some cases, the inducible promoter is a tetracycline inducible promoter. In some aspects, the invention provides methods to produce iPS cells modified to overexpress MafA in addition to Pdx1 and Ngn3. The mafA gene may be stably integrated in the iPS cell genome or may be delivered transiently before, after or during reprogramming.

In some aspects of the invention, a reporter molecule is also stably introduced into the iPS cells. In some cases, the reporter molecule in under the control of a promoter expressed in pancreatic endocrine progenitor cells but or derivatives thereof not expressed in primitive endoderm. In some cases the promoter is an ins1 promoter and the reporter molecule is a bla gene. In some cases, the reporter expression construct is stably integrated into the iPS genome. In some cases, the reporter expression construct is integrated into the ins1 locus. In some cases, the reporter expression construct is targeted by homologous recombination. In some cases the reporter expression construct is targeted by using a recombinase system; for example, a cre-lox recombination system. In some cases, the reporter expression construct is introduced into iPS cells before the pdx1 and ngn3 genes are introduced into the iPS cells. In some cases reporter expression construct is introduced into iPS cells after the pdx1 and ngn3 genes are introduced into the iPS cells. In some cases, the reporter expression construct is introduced into iPS cells at the same time as the pdx1 and ngn3 genes are introduced into the iPS cells. In some cases, reporter expression constructs are introduced to differentiated cells prior to reprogramming to iPS cells. In some cases, reporter expression constructs are introduced to iPS cells after reprogramming. In some cases, reporter expression constructs are introduced to cells during the reprogramming process.

Once an iPS cell is modified to overexpress Pdx1 and Ngn3, the stable integration of the pdx1 and ngn3 genes can be verified by methods known in the art. For example, PCR can be used to check proper integration of the pdx1 and ngn3 genes into a targeted integration site. Expression of the pdx1 and ngn3 genes following induction can be detected by RT-PCR. Immunohistochemistry can also be used to show expression of Pdx1 and Ngn3 in cells following induction. Likewise, stable integration of mafA gene can be verified by methods known in the art.

### IX. Methods of Use

### Screening

Pancreatic endocrine progenitor cells and/or primitive beta-islet cells of this invention can be used to screen for agents that affect the characteristics of pancreatic endocrine progenitor cells and their various progeny. The agent to be tested may be natural or synthetic, one compound or a mixture, a small molecule or polymer including polypeptides, polysaccharides, polynucleotides and the like, an antibody or fragment thereof, a compound from a library of natural or synthetic compounds, a compound obtained from rational drug design, a polynucleotide identified by microarray analysis, or any agent the effect of which on the cell population may be assessed using assays known in the art.

In some aspects of the invention, pancreatic endocrine progenitor cells and/or primitive beta-islet cells are used to screen the effect of agents that have the potential to up- or down-regulate insulin synthesis or secretion. The cells are combined with the test agent, and then monitored for change in expression or secretion rate, for example, by RT-PCR or immunoassay of the culture medium. In some aspects of the invention, the cells are combined with the test agent and then monitored for change in expression of a reporter gene. For example, in a screen of agents that may induce insulin secretion, pancreatic endocrine progenitor cells of the invention, in which a reporter gene operably linked to the ins1 promoter, is treated with the test agent. The potential of the agent to induce insulin secretion is then assessed based on the expression of the reporter gene. In some aspects of the invention, the cells are combined with the test agent and then monitored over time to evaluate the effect of the agent at specific times following introduction. For example, pancreatic endocrine progenitor cells of the invention are contacted with an agent and then monitored over time to determine the effect of the compound on the differentiation of the pancreatic endocrine progenitor cell into mature pancreatic cells; for example, mature β-islet cells.

The invention also provides methods for identifying genes involved in differentiation and development of pancreatic cells. For example, pancreatic endocrine progenitor cells, generated by overexpression of Pdx1 and Ngn3, are cultured and after different periods of time in culture, gene expression profiles of different populations are compared to identify genes that are uniquely expressed in a population. In some cases, additional genes are expressed or overexpressed at various times after induction of Pdx1 and Ngn3. In some aspects of the invention, microarray analysis and subtractive hybridization are used to compare gene expression profiles.

### Cell Therapy

The present invention also provides methods for generating mammalian cells in vitro from pluripotent cells. For example, pancreatic endocrine precursor cells may be generated from ES cells by overexpression of Pdx1 and Ngn3. In some cases, cells may be further differentiated toward pancreatic endocrine cells; for example, insulin-producing pancreatic islet cells. In some cases, the insulin secreting cells may be generated from ES cells by overexpression of Pdx1 and Ngn3 and by overexpression of MafA either simultaneous with Pdx1 and Ngn3 overexpression or following Pdx1 and Ngn3 overexpression.

In some aspects, the cell populations of the present invention are useful for generating differentiated cells and tissues for cell replacement therapies. For example, pancreatic endocrine progenitor cells and/or primitive beta-islet cells that have been induced to secrete insulin may be useful in the treatment of diabetes. In some cases, the diabetes may be Type I diabetes. In some cases, the diabetes may be Type II diabetes. The suitability of the cell populations of the present invention for cell replacement therapy may be assessed by transplanting the cells into animal models of disorders that are associated with the destruction or dysfunction of a limited number of cell types.

In some aspects of the invention, pancreatic endocrine precursor cells may be generated from iPS cells by overexpression of Pdx1 and Ngn3. In some cases, cells may be further differentiated toward pancreatic endocrine cells; for example, insulin-producing pancreatic islet cells. In some cases, the insulin secreting cells may be generated from iPS cells by overexpression of Pdx1 and Ngn3 and by overexpression of MafA either simultaneous with Pdx1 and Ngn3 overexpression or following Pdx1 and Ngn3 overexpression. Autologous or allogeneic populations of iPS cell-derived pancreatic endocrine cells may be used in cell replacement therapies. In some aspects of the invention, differentiated cells from an individual may be cultured and reprogrammed to iPSC by the methods described above. The iPSC may subsequently be differentiated to pancreatic endocrine cells and then implanted back into the individual in order to provide a patient specific therapy. In other aspects, allogeneic iPSCs or iPSC-derived pancreatic endocrine cell lines are established for cell therapies.

### Compositions

The invention provides compositions of pancreatic endocrine progenitor cells and compositions of primitive beta-islet cells and their derivatives. Cells for therapeutic use are typically supplied in the form of a pharmaceutical composition, comprising an isotonic excipient prepared under sufficiently sterile conditions for human administration. Likewise, the invention provides the use of pancreatic endocrine progenitor cells and primitive beta-islet cells and their derivatives in the manufacture of medicaments for the treatment of conditions associated with pancreatic endocrine function.

General principles in medicinal formulation of cell compositions can be found in Cell Therapy Stem Cell Transplantation, Gene Therapy, and Cellular Immunotherapy, by G. Morstyn & W. Sheridan eds, Cambridge University Press, 1996.

### EXAMPLES

The following examples are provided to illustrate, but not to limit, the invention.

### Example 1

### Pdx1 and Ngn3 Induce Insulin mRNA Expression in Activin-Induced Endoderm EBs

### Material and Methods

### Growth and Differentiation of ES Cells

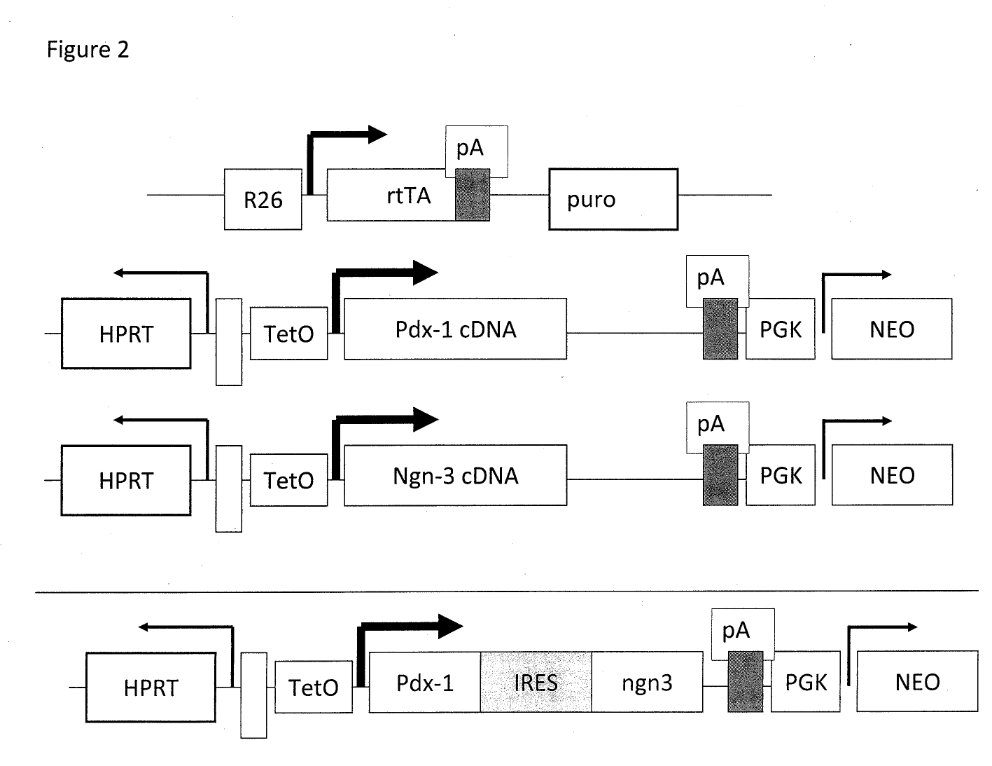

To assess the gene function in developmental progression of pancreas during ES cell differentiation, Ainv 18 ES cells were used. The cells can be used to target gene expression, which can be induced by exposure to doxycycline (Dox) (Sigma, St. Louis) at specific time points (Kyba, M. et al. 2002 Cell 109:29-37). Pdx1 or pdx1-IRES-ngn3 plox vectors (FIG. 2) were electroporated into Ainv 18 ES cells to yield Tet-pdx1 or Tet-pdx1/ngn3 ES cells. These cells can be induced to express Pdx1 or both Pdx1 and Ngn3 by Dox, respectively. ES cells were maintained on irradiated mouse embryo fibroblast feeder cells as previously described (Kubo, A. et al. 2004 Development 131:1651-1662). To generate embryoid bodies (EBs), ES cells were dissociated into a single cell suspension using trypsin and then cultured at various concentrations in 60 mm petri-grade dishes (Valmark) in differentiation media. Cultures were maintained in a humidified chamber under a 5% CO₂-air mixture at 37° C.

For differentiation of endoderm, activin induction was carried out using a two-step protocol (SP condition) (Kubo, A. et al. 2004 Development 131:1651-1662). First, to generate EBs, ES cells (4×10³ cells/ml) were incubated in Stem Pro 34 medium (Gibco) supplemented with 2 mM glutamine, 0.5 mM ascorbic acid, 4.5×10⁻⁴ M monothioglycerol (MTG) and c-kit ligand (1% conditioned medium). Second, the resultant EBs were harvested after 48 h of differentiation, allowed to settle in a 50 ml tube, transferred to new dishes and cultured in IMDM supplemented with 15% Knockout serum replacement (SR) (Gibco) supplemented with 2 mM glutamine, 0.5 mM ascorbic acid, 4.5×10⁻⁴ M MTG and human activin A (100 ng/ml) (R&D Systems). To induce pancreatic differentiation, Dox (1 μg/ml) in IMDM supplemented with 15% SR and 2 mM glutamine was introduced at day 6, for various durations. After a total of 10 days of differentiation, EBs were replated on Matrigel-coated 6-well dishes in IMDM supplemented with 15% fetal calf serum (FCS) (JRH) and 2 mM glutamine with or without Dox (1 μg/ml). Cells from these replated cultures were harvested at the indicated times (total differentiation time) for RNA isolation.

### Gene Expression Analysis

For reverse transcription-polymerase chain reaction (RT-PCR), total RNA was extracted using RNeasy mini-kits and then treated with RNase free DNase (Qiagen). One μg of total RNA was then reverse-transcribed to cDNA using a Superscript RT kit (Invitrogen) with random hexamers. PCR was carried using Taq polymerase (Takara Bio) in PCR buffer containing 2.5 mM MgCl₂ and 0.2 μM dNTPs. The amplification protocol entailed 1 cycle at 94° C. for 5 min followed by 25-40 cycles of 94° C. for 1 min (denaturation), 60° C. for 30 sec. (annealing) and 72° C. for 1 min (elongation), with a final elongation at 72° C. for 7 min. Oligonucleotide primers used for PCR were listed (Table 1).

For a real time PCR, commercially available assay mixes (Applied Biosystems) for Ins1 (Mm01259683_g1), Ins2 (Mm0731595_gH) and 18S (Hs99999901_s1) were used to quantify mRNA levels, and PCR was performed using a Prism 7700 Sequence Detector (Applied Biosystems). Ins1 and Ins2 mRNA levels were normalized to 18S mRNA levels in the same samples.

TABLE 1
Primer list for pancreas related-genes

| | |
|---|---|
| Forward | Reverse |

| | | |
|---|---|---|
| Ins1 | TAGTGACCAGCTATAATCAGAG | ACGCCAAGGTCTGAAGGTCC |
| Ins2 | CCCTGCTGGCCCTGCTCTT | AGGTCTGAAGGTCACCTGCT |
| Gcg | CAGAGGAGAACCCCAGATCA | TCATGACGTTTGGCAATGTT |
| Sst | GAGGCAAGGAAGATGCTGTC | AGTTCTTGCAGCCAGCTTTG |
| Ppy | GGCCCAACACTCACTAGCTC | CCAGGAAGTCCACCTGTGTT |
| Ghrl | GAAGCCACCAGCTAAACTGC | CGGATGTGAGTTCTTGCTCA |
| Gip | GCAAGATCCTGAGAGCCAAC | TTGTTGTCGGATCTTGTCCA |
| Glp1r | TCAGAGACGGTGCAGAAATG | CAAGGCGGAGAAAGAAAGTG |
| amy | CATTGTTGCACCTTGTCACC | TTCTGCTGCTTTCCCTCATT |
| Ela | GGAACCATCCTGGCTAACAA | CTCAGTTGGAGGCAATGACA |
| Alb1 | GCTACGGCACAGTGCTTG | CAGGATTGCAGACAGATAGTC |
| Afp | CCTGTGAACTCTGGTATCAG | GCTCACACCAAAGAGTCAAC |
| Fabp2 | GGAAAGGAGCTGATTGCTGTCC | CTTTGACAAGGCTGGAGACCAG |
| Shh | TTAAATGCCTTGGCCATCTC | CCACGGAGTTCTCTGCTTTC |
| Pcsk1 | TTGGCTGAAAGGGAAAGAGA | GCTTCATGTGCTCTGGTTGA |
| Pcsk2 | CTGTGACGGCTATGCTTCAA | AGCTGCAGATGTCCCAGAGT |
| Chga | GAGGAGGAAGAGGAGGCTGT | TGTCCTCCCATTCTCTGGAC |
| Glut2 | CGGTGGGACTTGTGCTGCTGG | CGCAATGTACTGGAAGCAGA |
| Gck | GCCTGTGTATGCAACCATTG | CATTTGTGGGGTGTGGAGTC |
| Kir6.2 | GGCTCCTAGTGACCTGCACCA | CCACAGCCACACTGCGCTTGCG |
| Foxa2 | TGGTCACTGGGGACAAGGGAA | GCAACAACAGCAATAGAGAAC |
| Ptfa1 | CACGCTACCCTACGAAAAGC | CCTCTGGGGTCCACACTTTA |
| Pax4 | AAATGGCGCAGGCAAGAGAA | ATGAGGAGGAAGCCACAGGA |
| Pax6 | GCTTCATCCGAGTCTTCTCCGTTAG | CCATCTTTGCTTGGGAAATCCG |
| NeuroD | CTTGGCCAAGAACTACATCTGG | GGAGTAGGGATGCACCGGGAA |
| Isl1 | AGATATGGGAGACATGGGCGAT | ACACAGCGGAAACACTCGATG |
| Nkx2.2 | AACCGTGCCACGCGCTCAAA | AGGGCCTAAGGCCTCCAGTCT |
| MafA | ATCATCACTCTGCCCACCAT | AGTCGGATGACCTCCTCCTT |
| Pdx1 | CCACCCCAGTTTACAAGCTC | TGTAGGCAGTACGGGTCCTC |
| Ngn3 | CTGCGCATAGCGGACCACAGCTTC | CTTCACAAGAAGTCTGAGAACACCAG |
| Hex | AAAAGGAAAGGCGGTCAAGT | CTGCTCACAGGAAGTGTCCA |
| β-actin | ATGAAGATCCTGACCGAGCG | TACTTGCGCTCAGGAGGAGC |

### Gene Overexpression Assay by Electroporation

Tet-pdx1 ES cells or Tet-pdx1/ngn3 ES cells were cultured in SP conditions. Day 6 EBs were dissociated with 0.25% trypsin/EDTA. The resulting cells (2×10⁶ cells) were suspended in mouse ES cell nucleofector solution (Amaxa). Pax4, Nkx×6.1 and Ngn3 were cloned into pIRES-EGFP vector (Clontech) and 5 μg of plasmids were electroporated into cells by Nucleofector device (ES solution, program O17) (Amaxa). Cells were washed and reaggregated in 24-well low-cluster dishes (Coaster) in SR media with Dox (1 μg/ml). EBs were harvested at day 8 for FACS and at day 9 for RNA isolation.

### Results

Pdx1 induces insulin mRNA in activin-induced endoderm EBs

To evaluate the role of Hex in hepatic specification in the ES cell/EB model, we used an ES cell line (AINV18) that enables the inducible expression of a given gene under the control of a tet-inducible promoter (Kyba, M. et al. 2002 Cell 109:29-37; Kubo, A. et al. 2005 Blood 105(12):4590-4597). Using a similar system, we evaluated factors that may be critical for pancreatic differentiation from ES cell-derived endoderm. Pdx1 is known to be a master gene for early pancreatic development from gut tube and as a first step in producing inducible endocrine progenitor cells, we introduced a gene encoding Pdx1 under the control of a tetracycline inducible promoter. For this set of experiments, EBs were generated in SP conditions. EBs were cultured for 2 days in the absence of serum (SP34 media) or factors to allow differentiation to the epiblast stage of development (stage 1: days 0-2) (Kubo, 2004 #7). Following this initial culture, EBs were exposed to activin in serum-replacement (SR media) for 4 days to induce definitive endoderm (stage 2: days 2-6). The activin treated EBs were then cultured in SR media for 4 days (stage 3: days 6-10), and then replated onto a matrigel coated wells in 15% serum media for a further 4 days to induce the differentiation and maturation (stage 4: days 10-20). Pdx1 expression was induced in the cells by the addition of Dox (1 μg/ml) to the EB cultures only at days 6-22.

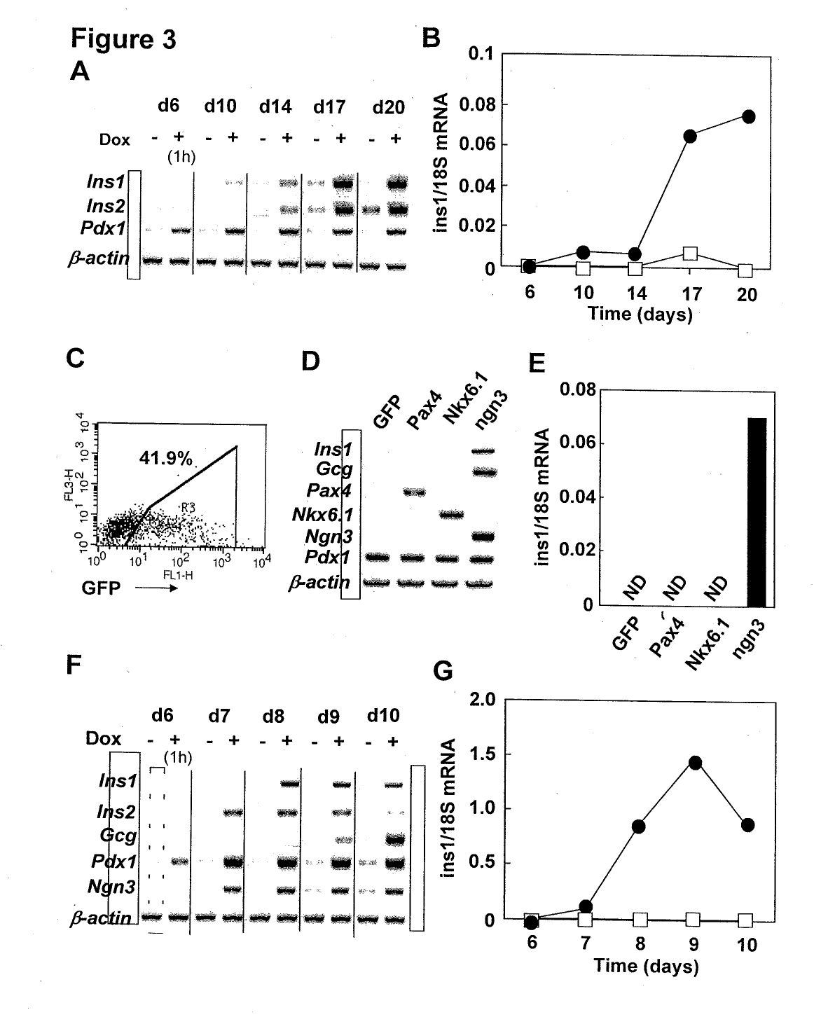

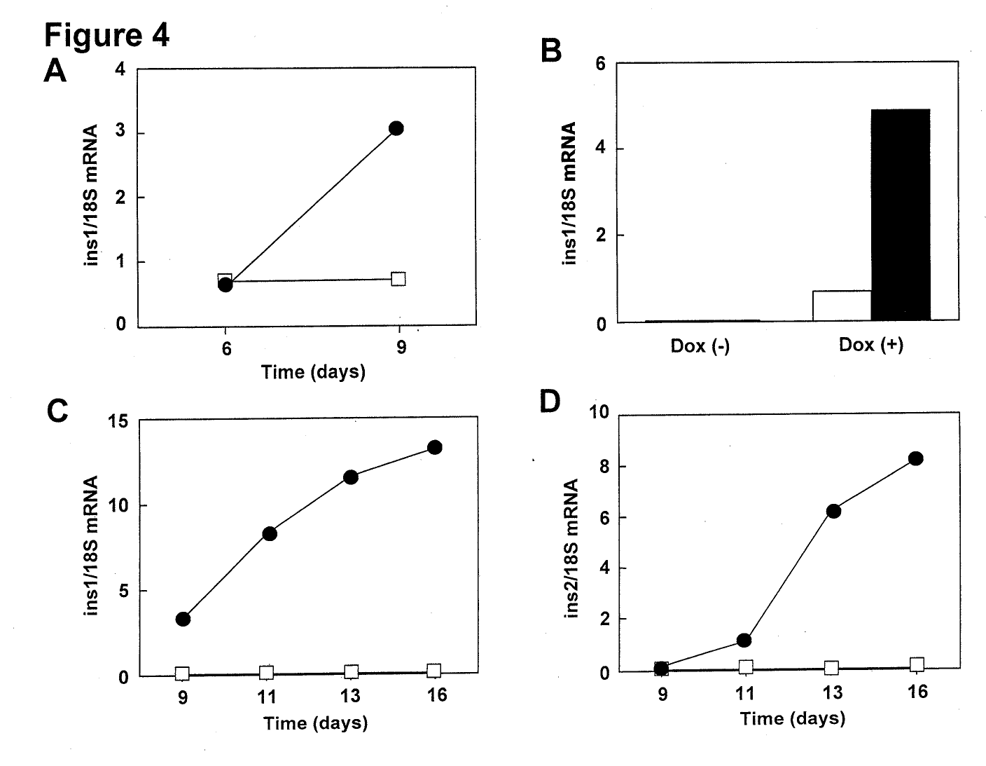

Gene expression of Pdx1 induced by Dox was confirmed by RT-PCR throughout the differentiation process (FIG. 3A). The induction of Pdx1 between days 6 and 22 of culture resulted in a significant upregulation of Ins1 and Ins2 mRNA expression at day 17 (FIG. 3A). Quantitative PCR analysis revealed that these levels of expression represented 0.08% of the expression found in insulinoma cell line, βTC6 (FIG. 3B). We also determined Ins1 mRNA levels at islet isolated from mouse pancreas. Ins1 mRNA levels are around 80-140% to that of βTC6.

Co-expression of Ngn3 with Pdx1 induces higher levels of insulin mRNA in activin-induced endoderm EBs.

Since Ins1 mRNA levels are very low compared with βTC6 or islet cells, we evaluated additional factors to improve β-cell differentiation from ES cells. As a quick screening system, we transiently expressed target genes using a pIRES2-EGFP vector by electroporation. We confirmed that this method could induce GFP expression in around 40% of cells as measured by FACS in EBs after 2 days of electroporation (FIG. 3C). Using this system, we induced gene overexpression of Pax4, Nkx×6.1 and Ngn3, which are all known to be important for β-cell specification. RT-PCR demonstrated that these genes are expressed at 3 days after electroporation (FIG. 3D). Surprisingly, only Ngn3 could induce Ins1 gene expression at significant levels by RT-PCR and by real time PCR at day 9 (FIG. 3D, E). The Ins1 mRNA levels at day 9 were comparable to that of day 17 EBs with Pdx1 expression. In order to create a stable ES cell line that could be induced to differentiate to pancreatic endocrine progenitor cells, we generated Ainv cells (Tet-pdx1/ngn3 ES cells) in which both Pdx1 and Ngn3 could be induced by Dox. When Dox was added at day 6, Ins1 mRNA was increased to 1.5% of βTC6 at day 9. Similar to the temporal gene expression discussed above, gene expression of glucagon was evident by day 10 following induction by Pdx1 and Ngn3 (FIG. 3F). These data indicate that co-expression of Ngn3 with Pdx1 increases Ins1 mRNA levels around 20 times fold higher than that with Pdx1 alone and significantly shortens the timing of the peak of Ins1 mRNA expression from day 20 to day 9 (FIG. 3G).

### Example 2

### BMP4 Improved Gene Expressions of Ins1 Induced by Pdx1 and Ngn3 in Serum-Free Differentiated Media

### Materials and Methods

Differentiation in serum-free differentiation medium (SFD) was carried using SFD condition described by Gouon-Evans, V. et al. 2006 Nat. Biotechnol. 24(11):1402-1411. SFD consisted of 75% IMDM and 25% Ham's F12 medium (Gibco) supplemented with 0.5% N2 and 1% B27 (with RA) supplements (Gibco), 1% penicillin/streptomycin, 0.05% bovine serum albumin, 2 mM glutamine, 0.5 mM ascorbic acid and 4.5×10⁻⁴ M MTG. ES cells (2−4×10⁴ cells/ml) were cultured in SFD in 60 mm Petri-grade dishes. At day 2 of differentiation, EBs were dissociated with trypsin/EDTA and replated at density of 2−6×10⁴ cells/ml in SFD supplemented with activin A (50 ng/ml) in 60 mm petri-grade dishes. The day 4 EBs were dissociated with trypsin/EDTA and were reaggregated by culture at high density (5×10⁵ cells/ml) in 24-well low-cluster dishes (Coaster) in SFD supplemented with BMP-4 (50 ng/ml) (R&D Systems), bFGF (10 ng/ml) (R&D Systems), activin A (50 ng/ml) and with or without Dox (1 μg/ml). At day 6, EBs were replated on gelatin coated dishes for monolayer culture or in 12-well low-cluster dishes (Nunc) for floating EBs in SFD media, with or without Dox (1 μg/ml).

### Results

Tet-pdx1/ngn3 Ainv ES cells were cultured in SFD for 2 days and then activin was added for days 2-4 to induce endoderm differentiation. At day 4, EBs were cultured with BMP4, bFGF and activin. At this time point, EBs were treated with Dox to induce Pdx1 and Ngn3 expression. Without Dox treatment, Ins1 mRNA was not detected at day 6 or day 9. EBs that were treated with Dox at day 4 to induce Pdx1 and Ngn3 gene expression resulted in Ins1 mRNA levels that increased to 0.6% of βTC6 at day 6 (FIG. 4A). EBs that were treated with BMP4 for days 4-6 and with Dox resulted in levels of Ins1 mRNA that further increased to 3.1% of βTC6 at day 9 (FIG. 4A). When day 6 EBs were replated on gelatin, some EBs attached to the plate to make a monolayer while other EBs continued to float and grow as floating EBs. Floating EBs were transferred to low-cluster dish at day 7. At day 9, Ins1 mRNA levels were higher in floating EBs than Ins1 mRNA levels in the monolayer cells, reaching to 4.9% of βTC6 (FIG. 4B).

In separate experiments, EBs were cultured with BMP4, bFGF and activin for days 4-6 and transferred to low-cluster dish at day 6 to maintain floating EBs until day 16. Dox was continuously added after day 4. Gene expression of Ins1 and Ins2 mRNA continued to increase until day 16 and the levels were 13.2% and 8.2% of βTC6, respectively (FIG. 4C,D). These data showed that the SFD condition improved Ins1 mRNA levels around 10 times fold compared to the SP condition.

### Example 3

### Pancreas Related-Genes are Induced by Pdx1 and Ngn3 in SFD Condition

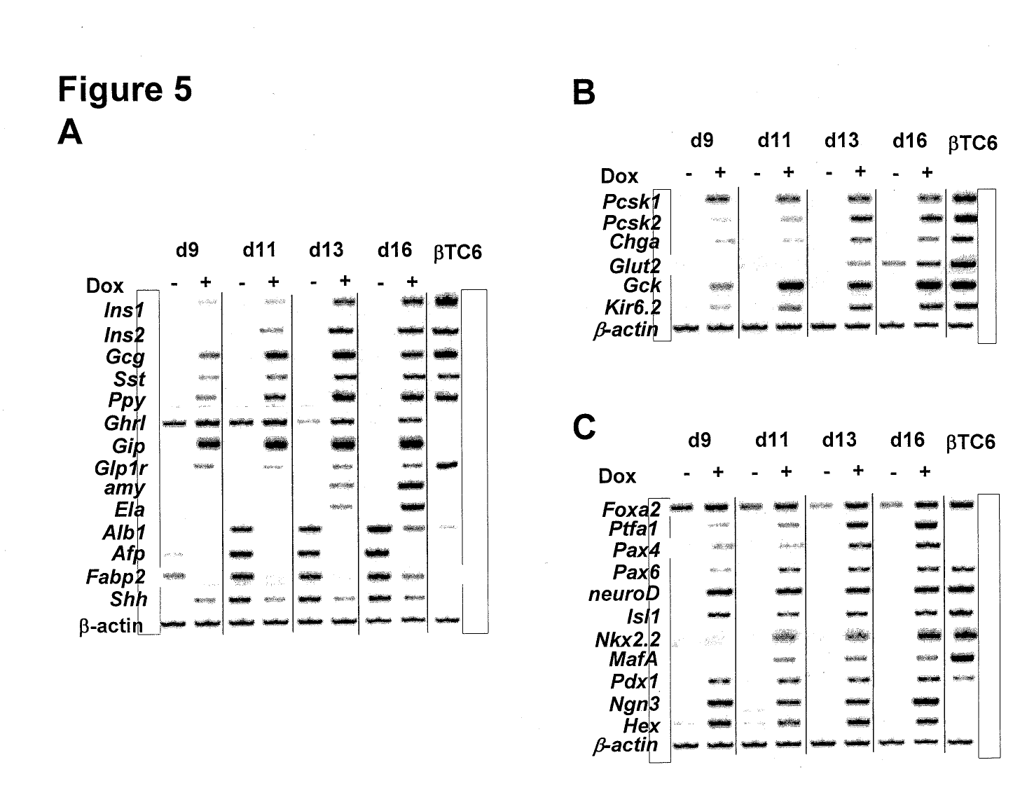

RT-PCR analysis demonstrated that overexpression of Pdx1 and Ngn3 in EBs induced a number of pancreas related-genes in addition to insulin (FIG. 5). Induced genes were categorized as follows; Secretory proteins (FIG. 5A): 1) pancreatic endocrine genes; Ins1, Ins2, Gcg, Sst, Ppy, and Ghrl. 2) Incretine hormone related-genes; Gip and Glp1r. 3) Exocrine genes; Amy and Ela. Liver and intestine related-genes such as Alb, Afp and Fabp2 are suppressed by Dox induction. Shh, which is important to be suppressed in pancreatic endoderm, was also suppressed by Dox induction. Insulin secretion related-genes (FIG. 5B): 1) insulin processing related-genes: Pcsk1, Pcsk2 and Chga. 2) glucose sensing related-genes: Glut2 and Gck. 3) potassium channel related-genes: Kir6.2. Pancreas related-transcriptional factors (FIG. 5C): Ptfa1, Pax4, Pax6, neuroD, Isl1, Nkx×2.2, MafA, and Hex. These results suggest that many important genes for pancreatic development and β-cell function are induced by Pdx1 and Ngn3 in SFD condition.

### Example 4

### Microarray Analysis of Genes Downstream of Pdx1 and Ngn3

For a more in depth analysis of the impact of Pdx1 and Ngn3 expression on lineage development, we carried out a microarray analysis (44) to identify genes activated downstream of these genes. For these studies, Tet-pdx1/ngn3 Ainv cells were differentiated in SFD condition with or without Dox and then day 13 EBs were compared by microarray analysis. In addition, E15.5 embryonic pancreas, adult islet and insulinoma cell line βTC6 were also evaluated by microarray as controls.

### Materials and Methods

For microarray analysis, total RNA was extracted using RNeasy mini kits (Qiagen), after which 10 μg of fragmented target total RNA was used for hybridization of each UniSet Mouse I Expression Bioarray chip (Amersham Life Sciences), which contained 10,012 probes. Once the microarrays were hybridized and washed, biotin-containing transcripts were directly detected using a Streptavidin-Alexa647 conjugate as previously described (Ramakrishnan et al., 2002). GeneSpring 6.2 (Silicon Genetics, Inc., Redwood City, Calif.) was then used to evaluate the data obtained using CodeLink™ Expression Scanning Software.

### Results

In this analysis, we demonstrated that variable pancreas-related factors are up-regulated by Pdx1 and Ngn3 induction (Table 2). These genes were categorized according to Gene Ontology (GO) analysis as follows; 1) extracellular: Genes in this category contain secretory proteins such as five pancreatic endocrine genes (Ins1 and 2, Sst, Gcg, Ppy, Ghrl), pancreatic exocrine gene (Cpa), genes related to insulin secretion (Scg, Chga, Pcsk) and enteroendocrine genes (Gip, Cck, Pyy, Sct). 2) Nuclear; Genes in this category contain transcriptional factors; β cell related transcriptional factors (Pax6, Insm1, Neurod1, Nkx×2.2, Isl1, Hhex, Nkx×6.1, Pax4) and β cell related transcriptional factors (Arx, Irx2). Functions of genes induced by Pdx1 and Ngn3 in another category (Cytoskeletal/membrane and Cytoplasmic/Signal) are currently unclear. Some genes (Dcx, Stmn2, Tubb3) in these categories were consistent with a previous study which evaluated novel effectors by Ngn3 using ES cells (Serafimidis, I. et al. 2008 Stem Cells 26(1):3-16).

TABLE 2
Pancreas-related factors upregulated by Pdx1 and Ngn3 induction.

| | SFD day 13 | | | | | |
|---|---|---|---|---|---|---|
| Gene | | | Dox +/− | | E15.5 | |
| Symbol | Dox (−) | Dox (+) | ratio | βTC6 | pancreas | islet |

Extracellular

| | | | | | | | |
|---|---|---|---|---|---|---|---|
| Sst | NM_009215 | 0.27 | 111.3 | 412.4 | 220.0 | 26.3 | 288.5 |
| Gip | NM_008119 | 0.62 | 251.2 | 402.7 | 0.5 | 3.6 | 0.4 |
| ins1 and 2 | | 0.27 | 73.6 | 272.5 | 375.6 | 227.1 | 281.3 |
| Scg3 | NM_009130 | 0.57 | 140.4 | 245.0 | 249.7 | 8.9 | 263.2 |
| Cck | NM_031161 | 0.74 | 175.9 | 238.1 | 365.8 | 6.8 | 0.3 |
| Pyy | NM_145435 | 1.76 | 221.9 | 126.1 | 6.1 | 128.1 | 288.8 |
| Cart | NM_013732 | 0.27 | 33.8 | 125.3 | 54.1 | 6.7 | 8.3 |
| Gcg | NM_008100 | 0.44 | 41.5 | 93.8 | 136.7 | 95.0 | 310.2 |
| Scg2 | NM_009129 | 0.48 | 43.7 | 91.4 | 241.0 | 7.7 | 309.2 |
| Resp18 | NM_009049 | 0.27 | 23.4 | 86.6 | 234.9 | 1.3 | 174.1 |
| Scg5 | NM_009162 | 0.39 | 32.1 | 81.3 | 74.8 | 4.4 | 97.3 |
| Chga | NM_007693 | 1.93 | 105.4 | 54.6 | 238.7 | 15.2 | 288.2 |
| Sct | NM_011328 | 3.10 | 116.4 | 37.6 | 398.2 | 1.9 | 0.3 |
| Cpa1 | NM_025350 | 0.46 | 15.1 | 32.5 | 0.3 | 216.9 | 262.5 |
| Gdf6 | NM_013526 | 0.97 | 28.1 | 29.1 | 0.3 | 2.6 | 0.3 |
| Ptprn | NM_008985 | 2.20 | 61.8 | 28.1 | 169.1 | 5.1 | 109.1 |
| Pcsk2 | NM_008792 | 2.17 | 60.9 | 28.1 | 195.4 | 12.9 | 180.4 |
| Fgf12 | NM_010199 | 0.33 | 7.6 | 23.1 | 30.2 | 1.6 | 11.5 |
| Chgb | NM_007694 | 0.27 | 6.2 | 22.8 | 35.3 | 1.6 | 9.9 |
| Cpa2 | NM_1024698 | 0.89 | 15.3 | 17.3 | 10.9 | 216.5 | 274.1 |
| Ppy | NM_008918 | 1.08 | 10.3 | 9.6 | 62.5 | 6.4 | 260.8 |
| Ghrl | NM_021488 | 4.08 | 34.9 | 8.5 | 0.4 | 20.1 | 4.4 |
| Pcsk1 | NM_013628 | 0.46 | 3.6 | 7.9 | 19.8 | 2.4 | 45.5 |

Nuclear

| | | | | | | | |
|---|---|---|---|---|---|---|---|
| Pax6 | NM_013627 | 0.28 | 36.2 | 127.8 | 95.2 | 9.59 | 62.5 |
| Arx | NM_007492 | 0.28 | 27.6 | 97.9 | 0.4 | 3.29 | 7.0 |
| Insm1 | NM_016889 | 0.27 | 24.6 | 90.9 | 73.0 | 8.15 | 52.9 |
| Myt1 | NM_008665 | 0.40 | 25.9 | 65.4 | 52.5 | 9.92 | 23.5 |
| St18 | NM_173868 | 0.27 | 15.1 | 55.9 | 21.1 | 2.05 | 23.7 |
| Neurod1 | NM_010894 | 0.62 | 30.6 | 49.4 | 64.6 | 4.07 | 34.8 |
| Nhlh2 | NM_178777 | 0.27 | 12.1 | 44.8 | 0.7 | 0.35 | 0.3 |
| Tnrc4 | NM_172434 | 0.27 | 10.9 | 40.5 | 5.0 | 0.65 | 2.8 |
| Elavl4 | NM_1038698 | 0.27 | 9.1 | 33.7 | 26.6 | 1.19 | 7.5 |
| Nkx2-2 | NM_010919 | 0.27 | 8.9 | 32.9 | 22.1 | 8.96 | 13.8 |
| Ebf3 | NM_010096 | 0.31 | 9.4 | 30.4 | 0.6 | 2.09 | 0.3 |
| Isl1 | NM_021459 | 1.61 | 41.3 | 25.7 | 120.5 | 12.40 | 43.9 |
| Lmo1 | NM_057173 | 0.67 | 12.3 | 18.4 | 40.4 | 1.64 | 1.9 |
| Hhex | NM_008245 | 0.60 | 7.9 | 13.1 | 0.3 | 3.70 | 1.9 |
| Irx2 | NM_010574 | 0.33 | 4.0 | 12.1 | 1.6 | 0.93 | 4.1 |
| Nkx6-1 | NM_144955 | 0.32 | 3.6 | 11.1 | 158.9 | 38.90 | 193.7 |
| Id4 | NM_031166 | 0.27 | 2.7 | 10.1 | 0.8 | 0.88 | 0.3 |
| Pou3f2 | NM_008899 | 0.27 | 2.6 | 9.5 | 0.3 | 0.30 | 0.3 |
| Uncx4.1 | NM_013702 | 0.27 | 2.6 | 9.4 | 0.3 | 0.30 | 0.3 |
| Ebf1 | NM_007897 | 1.20 | 8.0 | 6.6 | 1.1 | 4.98 | 1.8 |
| Bhlhb5 | NM_021560 | 0.27 | 1.6 | 5.8 | 0.3 | 0.30 | 0.3 |
| Pax4 | NM_011038 | 4.13 | 16.9 | 4.1 | 4.2 | 5.94 | 2.9 |

Cytoskeletal/membrane

| | | | | | | | |
|---|---|---|---|---|---|---|---|
| Dcx | NM_010025 | 0.27 | 43.9 | 162.6 | 68.5 | 5.48 | 4.56 |
| Stmn3 | NM_009133 | 0.27 | 38.0 | 140.5 | 73.9 | 1.11 | 8.1 |
| Stmn2 | NM_025285 | 0.29 | 37.9 | 129.0 | 9.1 | 5.60 | 6.05 |
| Stmn4 | NM_019675 | 0.27 | 33.1 | 122.4 | 8.2 | 0.54 | 0.51 |
| Astn1 | NM_007495 | 0.27 | 24.8 | 92.0 | 35.9 | 1.05 | 0.88 |
| Drd1ip | NM_026769 | 0.27 | 22.8 | 84.4 | 19.4 | 0.56 | 3.83 |
| Ecel1 | NM_021306 | 0.27 | 18.5 | 68.3 | 15.3 | 2.69 | 0.30 |
| Chodl | NM_139134 | 0.32 | 21.6 | 68.1 | 0.6 | 7.36 | 0.60 |
| Rimbp2 | XM_132396 | 0.84 | 42.1 | 50.4 | 155.9 | 21.29 | 97.51 |
| Mmd2 | NM_175217 | 0.32 | 16.2 | 50.2 | 31.1 | 7.59 | 0.88 |
| Lin7a | NM_1033223 | 0.30 | 13.3 | 43.7 | 7.3 | 0.82 | 0.73 |
| Tubb3 | NM_023279 | 0.27 | 11.6 | 42.9 | 3.2 | 0.43 | 0.7 |
| Dner | NM_152915 | 0.27 | 10.9 | 40.4 | 6.1 | 0.35 | 4.11 |
| Dpp6 | NM_010075 | 0.35 | 13.3 | 38.4 | 10.2 | 0.77 | 2.13 |
| Mast1 | NM_019945 | 0.31 | 11.4 | 36.3 | 1.7 | 0.43 | 3.44 |
| Glra2 | NM_183427 | 0.27 | 9.5 | 35.3 | 0.3 | 0.30 | 0.30 |
| Pld5 | NM_176916 | 0.34 | 11.7 | 34.2 | 5.2 | 0.63 | 0.52 |
| Sez6l2 | NM_144926 | 1.72 | 58.1 | 33.8 | 153.5 | 11.51 | 106.84 |
| Tmem27 | NM_020626 | 1.61 | 47.8 | 29.6 | 67.8 | 9.95 | 118.91 |
| Gcgr | NM_008101 | 0.76 | 15.5 | 20.3 | 0.3 | 1.19 | 16.69 |
| Dcx | NM_010025 | 0.27 | 43.9 | 162.6 | 68.5 | 5.48 | 4.56 |

Cytoplasmic/Signal

| | | | | | | | |
|---|---|---|---|---|---|---|---|
| Gng3 | NM_010316 | 0.32 | 42.2 | 130.6 | 10.4 | 2.21 | 2.7 |
| Calb1 | NM_009788 | 0.27 | 33.7 | 125.0 | 6.9 | 1.06 | 40.3 |
| Dcamkl1 | NM_019978 | 0.27 | 18.0 | 66.5 | 14.1 | 0.86 | 3.3 |
| Cryba2 | NM_021541 | 0.32 | 18.8 | 58.7 | 91.8 | 19.73 | 30.6 |
| Celsr3 | NM_080437 | 0.27 | 14.9 | 55.3 | 9.1 | 2.08 | 12.9 |
| Lin7a | NM_001033223 | 0.30 | 13.3 | 43.7 | 7.3 | 0.82 | 0.7 |
| Grin3a | XM_205495 | 0.40 | 16.6 | 41.7 | 0.7 | 3.54 | 0.3 |
| Sncg | NM_011430 | 0.27 | 9.1 | 33.5 | 0.3 | 1.63 | 13.8 |
| Plcxd3 | NM_177355 | 0.27 | 8.5 | 31.4 | 17.0 | 1.27 | 7.7 |
| Gck | NM_010292 | 2.42 | 25.4 | 10.5 | 10.9 | 8.51 | 31.7 |

### Example 5

### Pancreatic Population with Insulin Expression was Derived from CXCR4/c-kit^{+/+}

### Materials and Methods

### FACS Analysis and Cell Sorting

EB-derived cells prepared in SFD conditions were stained with a PE-conjugated anti-c-kit antibody (BD Pharmingen) and biotinylated rat anti-mouse CXCR4 antibody (BD Pharmingen) and visualized by streptavidin PE-Cy5 (BD Pharmingen). For insulin cytoplasmic staining, day 18 EBs were dissociated by 0.25% trypsin/EDTA and 0.05% collagenase. Cells were stained with an anti-insulin antibody (Dako, A0564) and visualized using a PE-conjugated anti-guinea pig IgG secondary antibody (Jackson Immunoresearch) using Cytofix/Cytoperm kit (Becton Dickenson) according the manufacturer's instruction. The stained cells were analyzed using a FACSan (Becton Dickenson, San Jose, Calif.) or sorted on a FACS Aria cell sorter (Becton Dickenson).

### Results

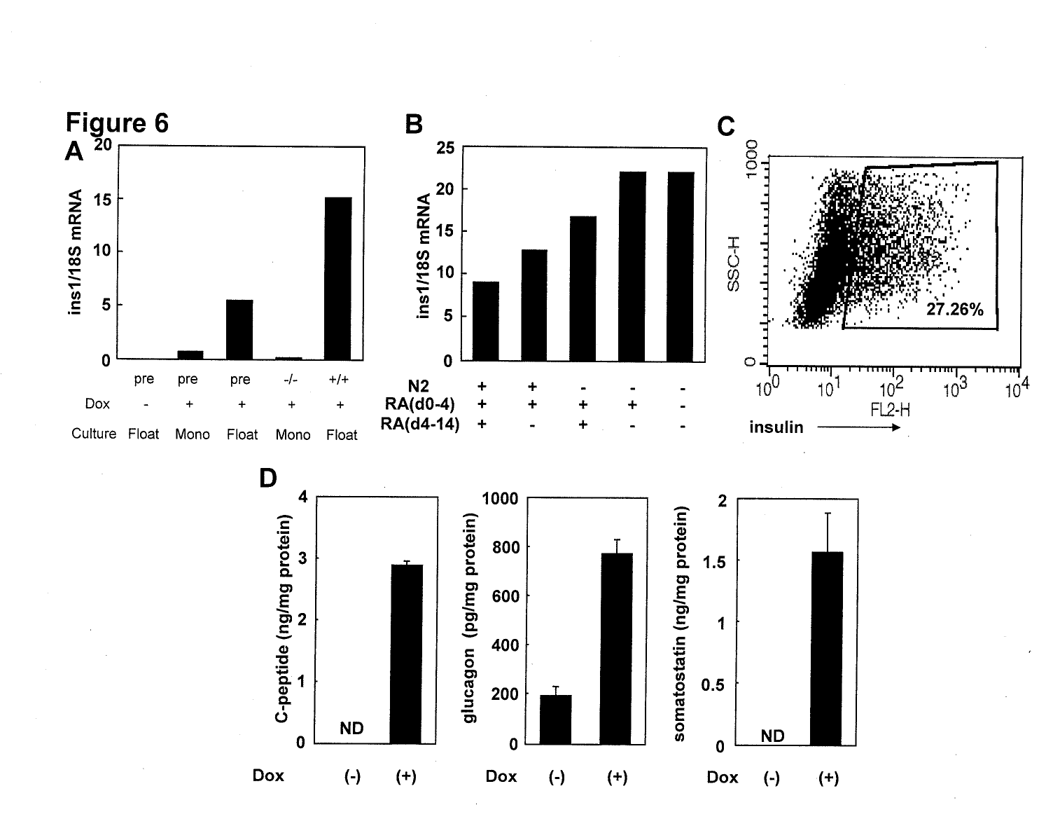

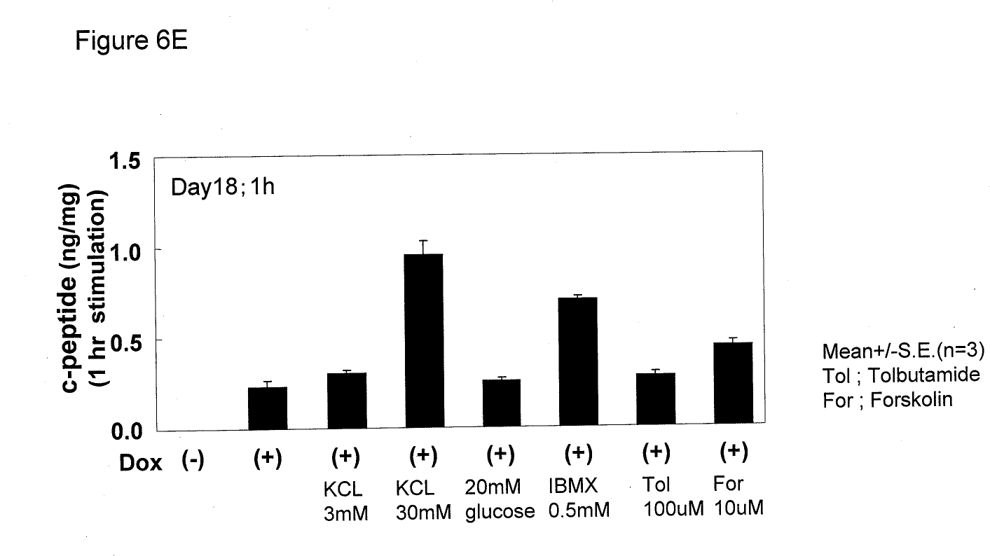

When CXCR4/c-kit^{-/-} cells were sorted by FACS, sorted cells were reaggregated and replated on gelatin coated dishes at day 6. Most cells from CXCR4/c-kit^{-/-} population attached on the gelatin coated dishes, whereas most of CXCR4/c-kit^{+/+} cells did not attach on gelatin coated dishes and keep floating. At day 9, Ins1 mRNA was not detected in monolayer cells from CXCR4/c-kit^{-/-} (FIG. 6A). On the other hand, Ins1 mRNA levels in EBs from CXCR4/c-kit^{+/+} cells was 2-fold higher than those in the floating EBs from pre-sort (FIG. 6A). These results suggest that pancreatic differentiation is also derived from CXCR4/c-kit^{+/+} definitive endoderm population. However, apoptosis-like cells appeared outside the floating EBs from CXCR4/c-kit^{+/+} cells, and EBs were getting small and disrupted after day 9.

### Example 6

### Optimization of SFD Conditions for Pancreatic Differentiation

The SFD condition contains a high concentration of insulin in the N2 supplement and RA in the B27 supplement. A recent study demonstrated that RA was important in the induction of pancreatic progenitor cells with Pdx1 (Micallef, S. J. et al. 2005 Diabetes 54:301-305). To optimize β-cell differentiation by Pdx1 and Ngn3 during ES differentiation, we evaluated if these components affected insulin gene induction during pancreatic EB differentiation. Depletion of N2 supplement and RA increases insulin mRNA to 23% of βTC6 (FIG. 6B). We also confirmed that cytoplasmic insulin staining by FACS was around 27% in EBs cultured in this condition with Dox stimulation (FIG. 6C), whereas only 0.3% cells were positive in EBs without Dox stimulation (data not shown). These data are comparable to that of insulin gene expressions by real time PCR.

### Example 7

### Analysis of Pancreatic Related Proteins by Immunohistochemistry

To evaluate if pancreatic related proteins were expressed in EBs induced by Pdx1 and Ngn3, immunohistochemical analysis was performed.

### Materials and Methods

### Immunostaining

For immunostaining, day 16 EBs, prepared under SFD conditions as described above, were replated on glass bottom dishes (Matek) coated by matrigel. Day 18 EBs were fixed in 4% paraformaldehyde for 20 min, washed two times in PBS, permeabilized in PBS with 0.2% triton-X100, washed in PBS with containing 10% FCS and 0.2% Tween 20, and then blocked for 10 min with PBS containing 10% horse serum. The cells were then incubated for 1 h with primary antibodies for insulin (Dako, A0564), C-peptide (Yanaihara, Y222), Pdx1 (Transgenic, KR059), Ngn3 (Santa Cruz sc-25655), Pcsk2 (Chemicon, AB1262) and Chga (Epitomics, #1782-1) and visualized using a Cy3-conjugated anti-guinea pig IgG secondary antibody or FITC-conjugated anti-rabbit IgG secondary antibody (Jackson Immunoresearch). After the second staining step, EBs were washed and then covered with antifade reagents with DAPI (Molecular Probe). Images were captured using an FLUOVIEW FV1000 confocal microscope (Olympus) with 10×, 40×, and 100× objectives.

### Results

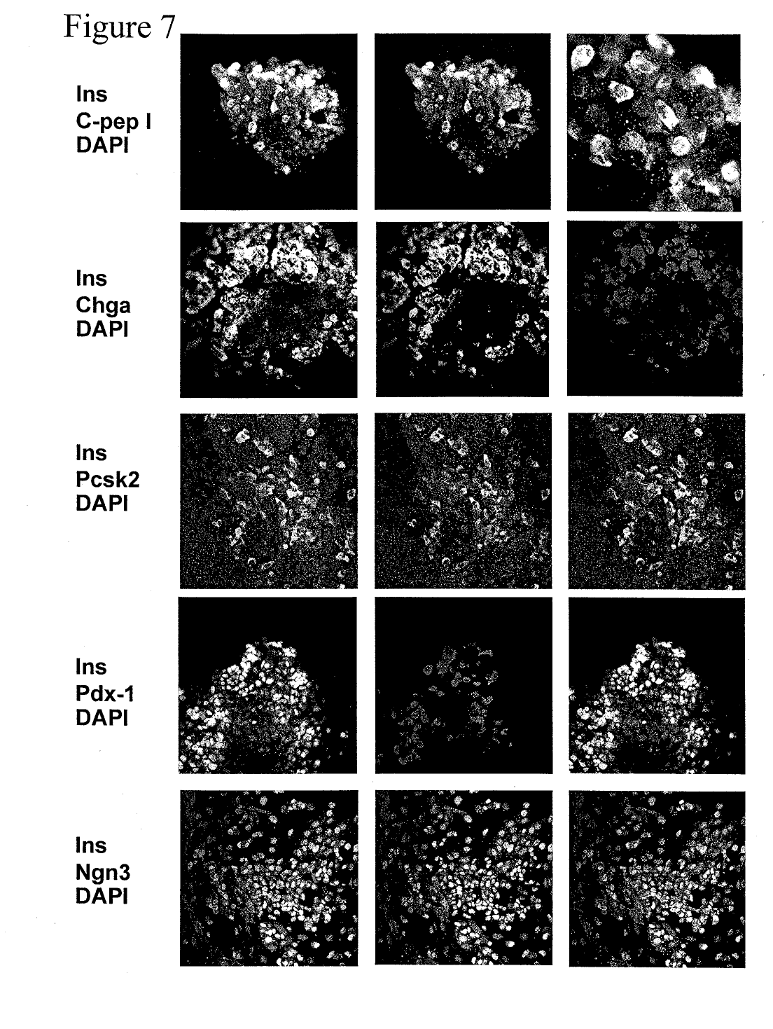

Tet-pdx1/ngn3 ES cells were cultured in SFD without N2 and RA for 16 days, with or without Dox, and replated on glass bottom dishes coated with matrigel. Day 18 EBs were stained by immunohistochemistry and analyzed by a confocal microscopy. Proteins such as insulin, C-peptide, Chga and Pcsk2 were expressed in EBs induced by Pdx1 and Ngn3 (FIG. 7), whereas no staining was detected in EBs without Dox stimulation (data not shown). Most insulin positive cells were co-expressed with C-peptide. We also detected Pdx1 and Ngn3 staining by Dox stimulation as the positive control. These results suggest that overexpression of Pdx1 and Ngn3 induces endocrine pancreas with β-cell related-proteins.

### Example 8

### C-peptide is Secreted in EBs Induced by Pdx1 and Ngn3 in SFD Condition

To evaluate if pancreatic related proteins were secreted in EBs induced by Pdx1 and Ngn3, immunoassay analysis of cell culture supernatants was performed.

### Materials and Methods

Measurement of C-Peptide, Glucagon and Somatostatin Secretion from EBs

After culturing EBs for 17-18 days in SFD conditions without N2 and RA with or without Dox (1 μg/ml) as described above, the medium was changed to fresh SFD media containing 2 mM glutamine. The EBs were then incubated for 24 hours as indicated, and the conditioned medium was collected for assay. Concentrations of glucagon and somatostatin in the conditioned medium were measured using enzyme immunoassays (EIAs) specific to glucagon (Yanaihara) or somatostatin (Phoenix Pharmaceuticals) according the manufacturer's instructions. C-peptide was measured by radioimmunoassay (RIA) specific to C-peptide (Linco). For C-peptide secretion assay, day 18 EBs were washed with media were incubated in HEPES-balanced Krebs-Ringer bicarbonate (HKRB) buffer (20 mM HEPES, 103 mM NaCl, 4.8 mM KCl, 0.5 mM CaCl₂, 1.2 mM MgSO₄, 1.2 mM KH₂PO₄, 25 mM NaHCO₃, 2 mM glucose, pH 7.4) with or without stimulations for 1 hour. C-peptide in the supernatant was measured by a specific RIA. Total protein amounts of EBs in each sample were evaluated by BCA assay and secretion levels for C-peptide, glucagons and somatostatin were adjusted by protein amount.

### Results

To evaluate pancreatic hormone secretion, pancreatic EBs were cultured in SFD without N2 and RA for 16-18 days and then EBs were incubated in fresh SFD media for 24 hours. The secretion of pancreatic hormones such as C-peptide, glucagon and somatostatin in the supernatant was evaluated by RIA or EIA. C-peptide, somatostatin and glucagons were not detected in EBs without Dox stimulation. These levels were significantly increased, however, in EBs with Dox stimulation (FIG. 6D). Stimulation of C-peptide secretion by treating endocrine progenitor cells with different agents for one hour was also evaluated (FIG. 6E). C-peptide secretion increased around five fold by the addition of 30 mM potassium chloride (KCl). Forskolin and IBMX, which increase intracellular cAMP, also stimulated C-peptide secretion around 2 fold and 3 fold, respectively. No response to glucose or the inhibitors of K_{ATP} channel, glibenclamide and tolbutamide, was detected. These results suggest that pancreatic EBs induced by Pdx1 and Ngn3 respond to direct stimulation such as a depolarization of cells by KCl or increase of intracellular cAMP. These EBs, however, did not have the machinery for the response to glucose or K_{ATP} channel inhibitor.

### Example 9

### Microarray Analysis of Insulin Expression

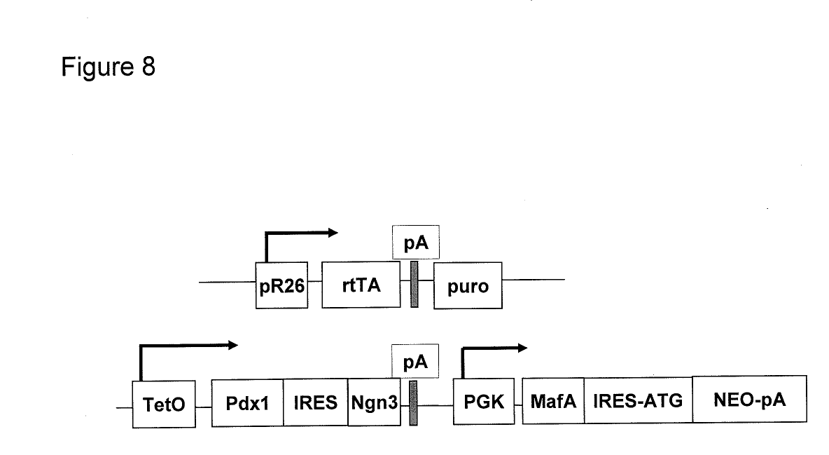

Parental Ainv cells were engineered, by means lox-mediated recombination, to conditionally express murine Pdx1, murine Ngn3, or the open reading frame of both cDNAs linked together by an EMCV IRES element (Pdx1/Ngn3) (FIG. 2). Parental Ainv cells contain the reverse tet transactivator (rtTA) inserted into the ROSA26 locus and a tet-regulated promoter inserted into the 5′ region of the HPRT locus. Downstream of the tet-regulated promoter is a lox site, followed by a 5′ truncated neomycin-resistance marker. Successful recombination into the lox site of the Ainv cells inserts the cDNA(s) of interest downstream of the tet-regulated promoter and reconstitutes the neo^{R} ORF, allowing selection using G418. For each cDNA construct tested, G418-resistant cells were isolated and used in subsequent pancreatic differentiation protocols. Triple-overexpression of Pdx1, Ngn3 and MafA was achieved using a strategy in which Pdx1 and Ngn3 were expressed from the tet-regulated promoter, while the MafA cDNA was constitutively expressed from the PGK promoter (FIG. 8).

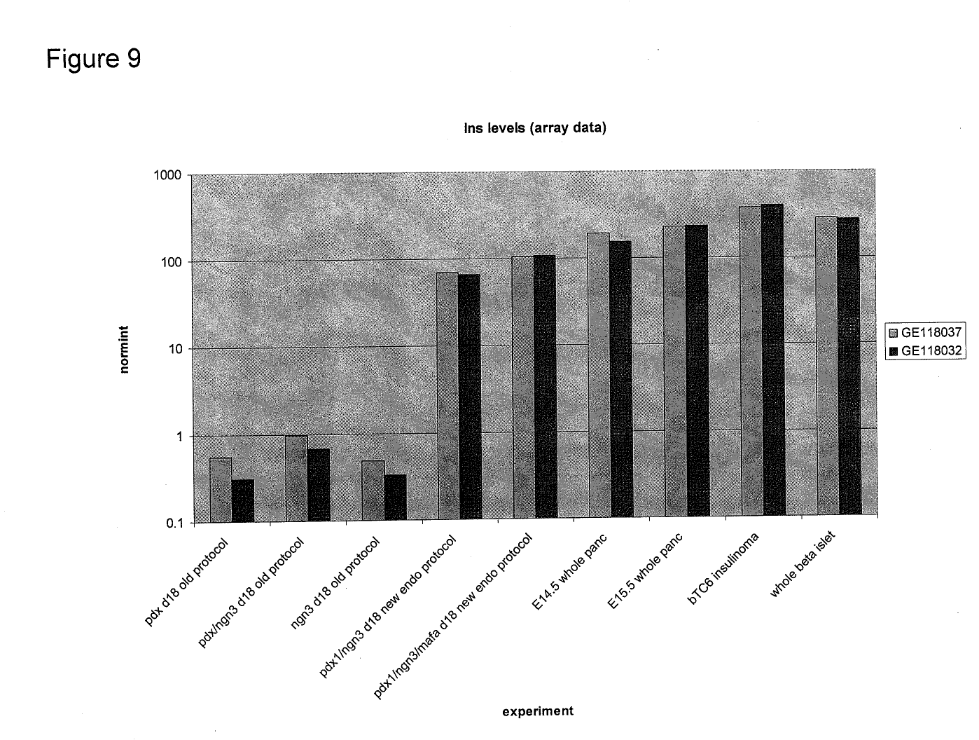

In some cases (labeled old protocol in FIG. 9), ES cells were differentiated using the following protocol. ES cells were maintained on MEF feeder cells for two days and then transferred to gelatin coated culture flasks for one to two days. The mES cells were partially differentiated at this point. To induce ES cells to form EBs, ES cells were removed from flasks with trypsin, counted, centrifuged, resuspended in SP-34 medium and plated on 60 mm plates. Cells were then incubated at 37° C. in 5% CO₂. On day 2, the media was removed from the plates and replace with SR medium containing activin A at a final concentration of 100 ng/ml. Cells were then incubated at 37° C. in 5% CO₂. On day 6, EBs were allowed to settle and the medium was replaced with Day 6 medium (85% IMDM, 15% Knockout serum replacement (SR) (Gibco) supplemented with 2 mM glutamine, 0.5 mM ascorbic acid, 4.5×10⁻⁴ M MTG) with or without Dox, final concentration 1 μg/ml). Cells were then incubated at 37° C. in 5% CO₂ for 12 days.

In some cases (labeled new endo protocol in FIG. 9), ES cells were differentiated using the following protocol. ES cells were maintained on MEF feeder cells. Four days before induction of differentiation, cells were removed from culture by trypsin and resuspended in SFES Maintenance Medium (50% Neurobasal medium (Invitrogen/Gibco), 50% DMEM/F12 (Invitrogen/Gibco), 0.5× B27 without RA (Stem Cells Tech), 10% BSA (Invitrogen/Gibco), 1 mM L-glutamine, 5% LIF, 1.46×10⁻⁴ M MTG and 10 ng/ml BMP) and plated onto gelatinized T785 flasks. Cells were then incubated at 37° C. in 5% CO₂ for 2 days. Two days before differentiation, cells were passaged to yield a good density (˜1:2-1:5). On day 0, ES cells were induced to make EBs. Cells were removed from flasks by trypsinization, counted and centrifuged. Cell pellets were washed twice with IMDM and resuspended to a concentration of 1×10⁵ cells/ml in SFD Complete Medium (75% IMDM, 25% Ham's F12, 0.5× B27 without RA, 10% BSA (Albumax I, Invitrogen/Gibco), 4.5×10⁻⁴ M MTG, 1×L-glutamine, 50 μg/ml ascorbic acid) into 60 mM dishes. On day 2, cells from three dishes were pooled and disaggregated by treatment with trypsin. Cells were then passed twice through a 20½ gauge needle attached to a 5 ml syringe. Disaggregated cells were then counted, centrifuged and resuspended to a concentration of 2×10⁵ cells/ml in SFD Complete Medium supplemented with 50 ng/ml activin A and plated in 60 mM dishes. Cells were then incubated at 37° C. in 5% CO₂ for two days. On day 4, cells were removed from dishes by trypsinization and disaggregated by passing the cells through a 20½ gauge needle attached to a 5 ml syringe two times. Cells were then counted, centrifuged and resuspended in Reaggregation Medium (75% IMDM, 25% Ham's F12, 0.5× B27 without RA, 10% BSA (Albumax I, Invitrogen/Gibco), 4.5×10⁻⁴ M MTG, 1× L-glutamine, 50 μg/ml ascorbic acid, 10 ng/ml bFGF (R&D Systems), 50 ng/ml BMP-4 (R&D Systems) and 50 ng/ml activin A (R&D Systems)) without or with 1 μg/ml Dox. Cells were plated onto 24 well low attachment plates. Cells were then incubated at 37° C. in 5% CO₂ for two days. Cells from each treatment group (+ or − Dox) were pooled carefully so as not to disturb EBs. EBs were centrifuged at 1000 rpm for 3 min, washed with IMDM and resuspended in Day 6-16 Medium (75% IMDM, 25% Ham's F12, 0.5× B27 without RA, 10% BSA (Albumax I, Invitrogen/Gibco) and 1× L-glutamine) without or with 1 μg/ml Dox. Cells were then plated 1:1 in low attachment 12 well plates based on the number of wells that were pooled from the 24 well plates. Cells were then incubated at 37° C. in 5% CO₂ for three days. Cells were fed on days 9, 11 and 13 by pooling cells from same treatment groups, centrifuging at 1000 rpm for 3 min, removing the media by aspiration and resuspending in 2 ml/well Day 6-16 Medium with or without Dox. On day 16 cells were analyzed.

For reference samples, total RNA was obtained (1) from whole pancreas harvested from d14.5 or d15.5 embryonic mice using standard Trizol-based methods, (2) from βTC6 insulinoma cells lines using RNeasy kits from Qiagen, or (3) from intact β-islets harvested from adult mice.

Microarray target preparation for CodeLink Arrays was performed per manufacturer's instructions (CodeLink Express Assay Reagent Kit; GE Healthcare). Briefly, one microgram of total RNA from each sample was reverse-transcribed into cDNA using T7-(dT)24 primers, and biotinylated cRNA prepared from this cDNA template by in vitro transcription. Ten micrograms of fragmented, biotinylated cRNA was hybridized to each CodeLink Mouse Whole Genome Array for 18 hours at 37° C. Afterwards, arrays were washed in 75 mM Tris-HCL, pH 7.6, 113 mM NaCl, 0.0375% Tween-20 for 1 hour at 46°, then stained with a 1:500 dilution of streptavidin-Alexa 647 (Molecular Probes) for 30 min at room temperature. Following the staining, arrays were washed three times, 5 min each, at room temperature with 0.1M Tris-HCL, pH 7.6, 0.15 M NaCl, 0.05% Tween-20, then once with 0.1×SSC/0.05% Tween for 30 sec, then dried in a centrifuge. Processed arrays were scanned using a GenePix 4000B Scanner and GenePixPro v4 software (Axon Instruments). Images were analyzed using CodeLink Expression Analysis Software, and the raw intensity data exported into GeneSpring GX (Agilent Life Sciences), within which raw intensity signals for each probe were median normalized. Because some CodeLink probes were improperly annotated as to their intended target, refinement of gene-to-probe associations was accomplished by analysis using VistaGen's Fred™ knowledgebase which maps the genomic coordinates of probes with that of the exons of genes and provides various bioinformatics analytical and functional genomics tools. All genomic coordinates on the mouse genome build 36 were determined using BLAST. Invalid probes, such as the ones that target multiple regions or intergenic regions on the genome, were removed from subsequent analyses. Data shown in the FIG. 9 and Table 3 reflect the average normalized intensity for a given Ins probe from biological replicates (n=2) of the indicated samples.

TABLE 3
Microarray analysis of insulin expression

| | | pdx/ngn3 | | pdx1/ngn3 | pdx1/ngn3/mafa | | | | |
|---|---|---|---|---|---|---|---|---|---|
| | pdx d18 | d18 | ngn3 d18 | d18 | d18 | E14.5 | E15.5 | | |
| | old | old | old | new endo | new endo | whole | whole | bTC6 | whole |
| probe | protocol | protocol | protocol | protocol | protocol | panc | panc | insulinoma | beta islet |
| GE118037 | 0.55802 | 0.970094 | 0.486271 | 69.60451 | 105.47138 | 190.7303 | 227.1426 | 375.58075 | 281.2518 |
| GE118032 | 0.311016 | 0.682766 | 0.330206 | 65.890076 | 107.61138 | 153.9106 | 232.4269 | 396.40414 | 275.3854 |

### Example 10

### Development of a Mouse Embryonic Stem Cell-Based Screening Assay for Diabetes Drug Discovery

In order to develop of screening assay for diabetes drug discovery, engineered mouse embryonic stem cell lines were generated that incorporate two key elements: 1) β-lactamase as an insulin reporter that allows quantitative measurement of Ins1 message, and 2) tetracycline-regulatable overexpression of Pdx1 and Ngn3.

### Construction of an Ins1-BLA Vector

Genomic DNA (gDNA) was isolated from Ainv15-MK cells (on gelatin) using the Qiagen DNA Blood & Cell Culture Midi kit. The ins1 3′ targeting arm was isolated by PCR amplification of 820 ng of Ainv15-MK gDNA, using the Roche Extend Long Template System as follows: 5 μl buffer #1, 1.78 μl 10 mM dNTPs, 0.75 μl enzyme mix, 0.6 μl 25 μM forward primer 3-Ins1-Xba1-F (GACTGCTCTAGAcaaccgtgtaaatgccactg), and 0.6 μl 25 μM reverse primer 4-Ins1HindIII-R (GACTGCAAGCTTtgagcatccacctctgtgtt). The mixture was cycled in a BioRad iCycler PCR machine using the following program: 94° C. for 2 min; 10 cycles of 94° C. for 10 sec, 60° C. for 30 sec, 68° C. for 2 min; 25 cycles of 94° C. for 15 sec, 60° C. for 30 sec, 68° C. for 2 min and increasing by 5 sec each cycle; 68° C. for 7 min, and 4° C. dwell. A 2 kb

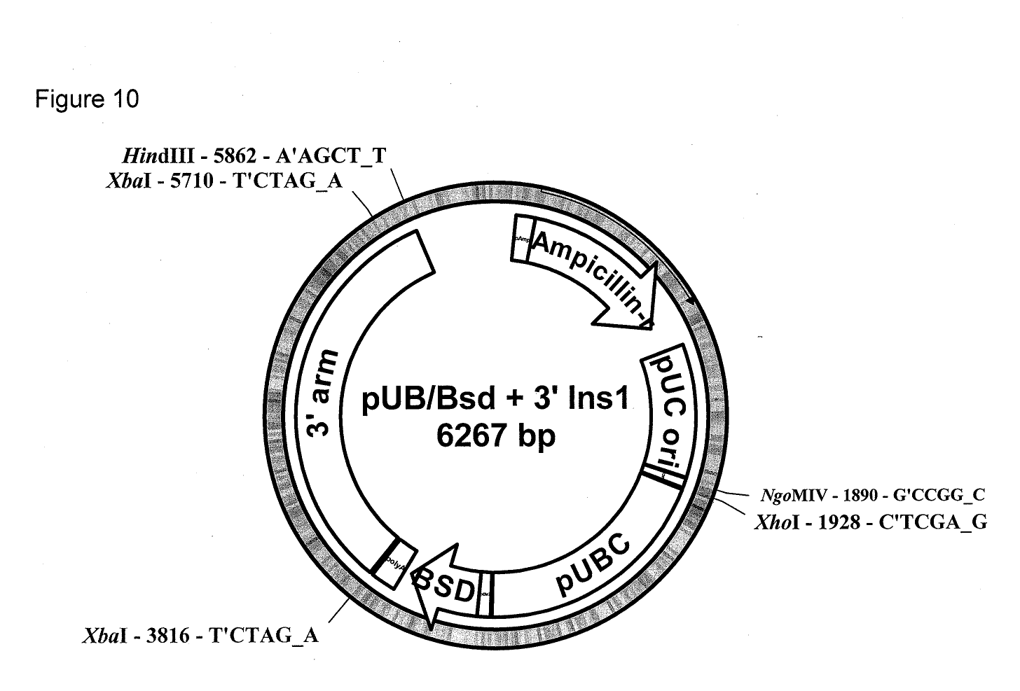

PCR product band was cut from the gel and DNA was isolated using BioRad Spin Columns. The 3′ targeting arm DNA was then digested with XbaI (partial) and HinDIII, gel purified, and isolated with the Zymo Gel DNA Recovery kit. It was then ligated into a BioRad spin column-purified pUB/Bsd backbone from which a 24 bp HinDIII-XbaI fragment had been excised. Clone #6 was confirmed by restriction digest and was the clone used for subsequent cloning steps. The resultant vector was designated Bsd+3′ Ins1 (FIG. 10).

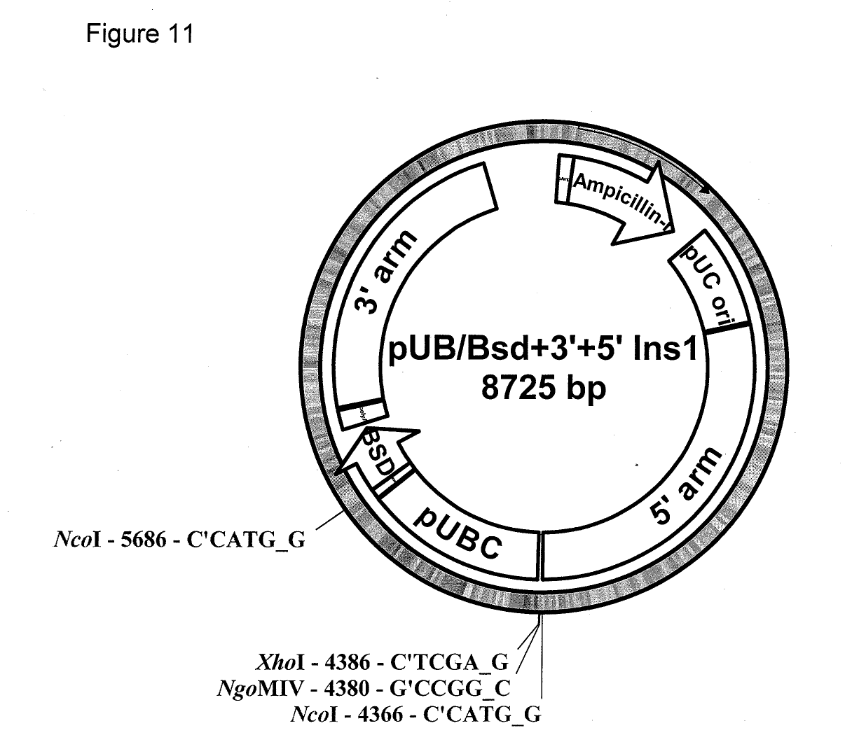

The Ins1 5′ targeting arm was isolated from Ainv15-MK gDNA by PCR amplification in the same manner as the 3′ arm, although Roche Expand High Fidelity Taq was substituted for Roche Expand Long Template Taq (the buffer remained the same). The forward primer was 1-Ins1-Xma1-F (GACATTCCCGGGacactggagaagggggttct), and the reverse primer was 2-Ins1-NNNX-Rshort (GACTGTCTCGAGGCCGGCGCGGCCGCCCATGGgcttgctgatggtctctg). A 2.5 kb PCR product band was gel purified using the Zymo Gel Recovery Kit. The 5′ targeting arm was digested with XmaI and XhoI and then cleaned with the Zymo Clean & Concentrator kit. This fragment was ligated to a Bsd+3′ Ins1 backbone that had been digested with XhoI and NgoMIV and gel purified with the Zymo Gel Recovery kit. DH5a cells were transformed with 5 μl of this ligation. Clone #6 was confirmed by restriction digest and was the clone used for subsequent cloning steps. The resultant vector was designated Bsd+3′+5′ (FIG. 11).

Bsd+3′+5′ was digested with NcoI (partial) and NgoMIV, and the linearized 8.7 kb band was gel purified using the Zymo Gel Recovery kit. BLA and its associated polyA were isolated from the pGeneBLAzer™ vector by NcoI/NgoMIV digestion. pGeneBLAzer encodes a mutated version of the bla designated bla(M). A 1.2 kb band was gel isolated and purified with the Zymo Gel Recovery kit. These two fragments were ligated and transformed into DH5a cells. Clone #11 was confirmed by restriction digest and was partially sequenced in the forward direction with the following primers:

Ins1bla1757: tgaccactgtgcttctgagg
Ins1bla2200: ggggaatgatgtggaaaatg
Inslbla5393: aggtgcttctcgatctgcat

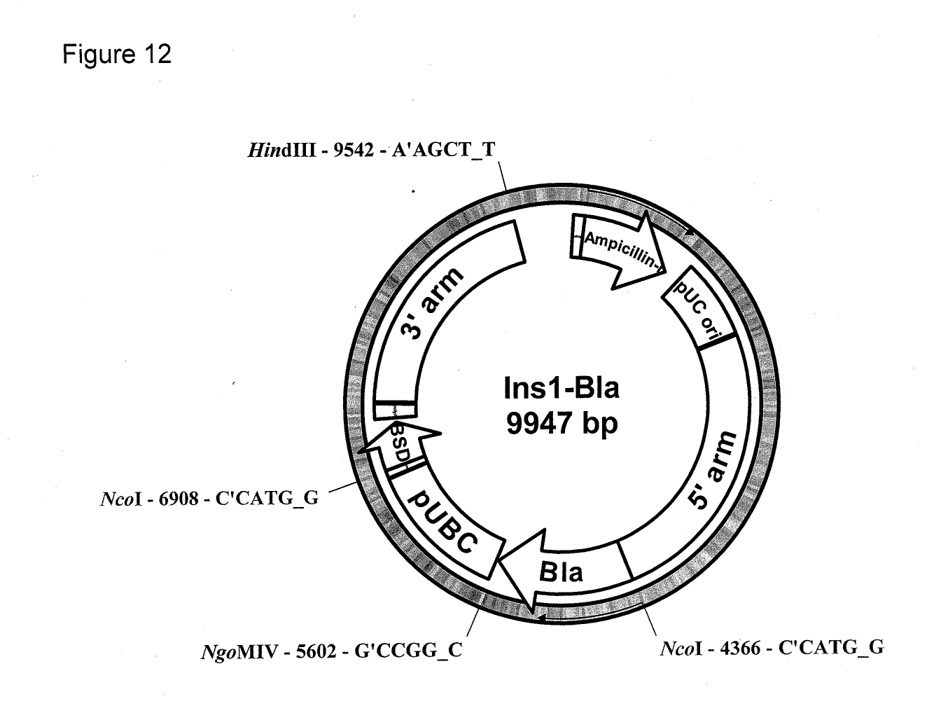

There were two point mutations (or polymorphisms) at 2184 bp (in 5′ arm) and 5829 bp (in 3′ arm); however, they don't appear to be in any known regulatory/promoter regions. Clone #11 was used for electroporation into Ainv15-MK mES cells. The resultant vector was designated Ins1-Bla (FIG. 12).

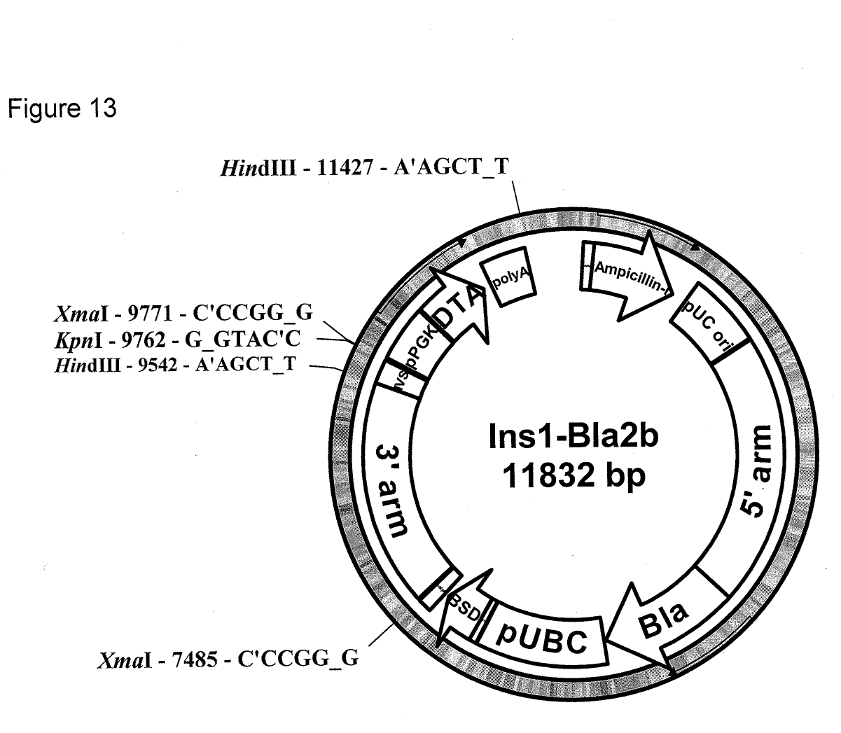

A diptheria toxin A (DTA) negative selection cassette was added to the Ins1-Bla vector as follows: The Ins1-Bla vector was digested with HinDIII and then treated with Antarctic Phosphatase. A 1.9 kb HinDIII fragment was excised from the TV.uni.puro.str vector, gel purified using the Zymo Gel Recovery kit, and then ligated to the HinDIII-digested Ins1-Bla backbone. DH5a cells were transformed with 5 ul of the ligation mix. Clones #3, #9, and #10 were confirmed by restriction digest. The resultant vector was designated Ins1-Bla2b (FIG. 13).

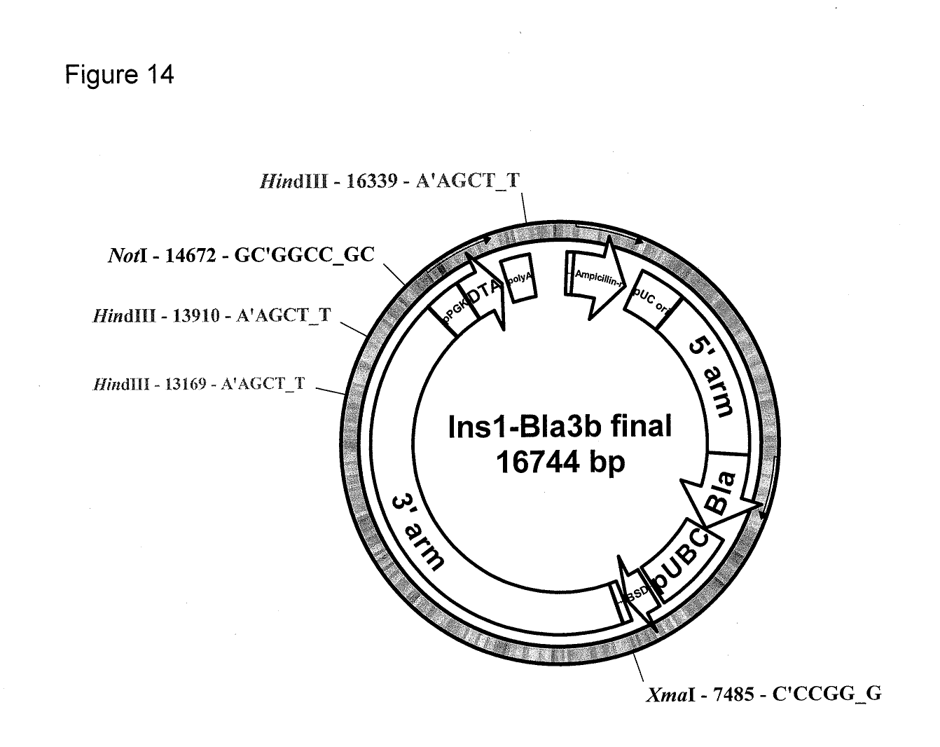

The 3′ targeting arm (2 kb) of the Ins1-Bla2b vector was replaced with a longer 3′ targeting arm (7.2 kb) as follows: The longer 3′ targeting arm was amplified from 500 ng Ainv15-MK gDNA in the same manner as the shorter 3′ arm had been isolated, although the base extension times were increased to 4.5 minutes and the dNTPs were decreased to 1.75 ul. The forward primer used was 3-Ins1-XmaI-Fb (gactgccccgggcaaccgtgtaaatgccactg), and the reverse primer used was 4-Ins1-XmaINot1 (GACTGCCCCGGGtcagctGCGGCCGCctgctgccatgactacctga). The PCR product was cleaned up with a Qiaquick PCR Purification kit, then digested with XmaI, and then cleaned up a second time. Ins1-Bla2b Clone #9 was digested with XmaI and then treated with Antarctic Phosphatase. A 9.5 kb backbone band was gel purified with the Zymo Gel Recovery kit and then ligated to the newly amplified longer 3′ targeting arm. 5 ul ligation mix was used to transform DH5a cells. Clone #2 was confirmed by restriction digest, except for the absence of a second XmaI site, and then sequenced with the following primers: Ins1bla3b_{—}4961(cagccaccattacaatgcac), Ins1bla3b_{—}5651 (tcaggtagtcatggcagcag), and Ins1bla5393 (aggtgcttctcgatctgcat). Sequencing confirmed that the XmaI site at the 3′ end of the 3′ targeting arm did not reconstitute during ligation. There is one basepair ‘missing’ from the beginning of the pPGK sequence, however, upon BLAST search it was determined that new sequences do not contain this basepair. Finally, there are two point mutations (or polymorphisms) and some extra repetitive CA's at the 3′ end of the 3′ targeting arm, however, this is not in a critical region and potentially may be a sequencing artifact. Ins1-Bla3b clone #2 (FIG. 14) was used for electroporation into Ainv15-MK mES cells after linearization with Not1 and ethanol precipitation.

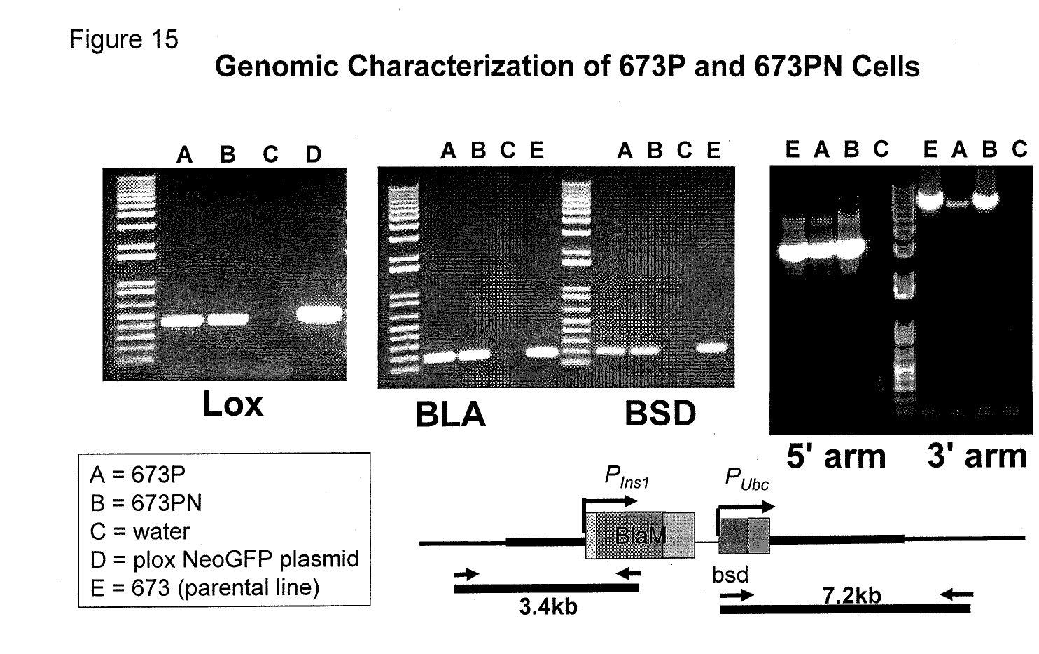

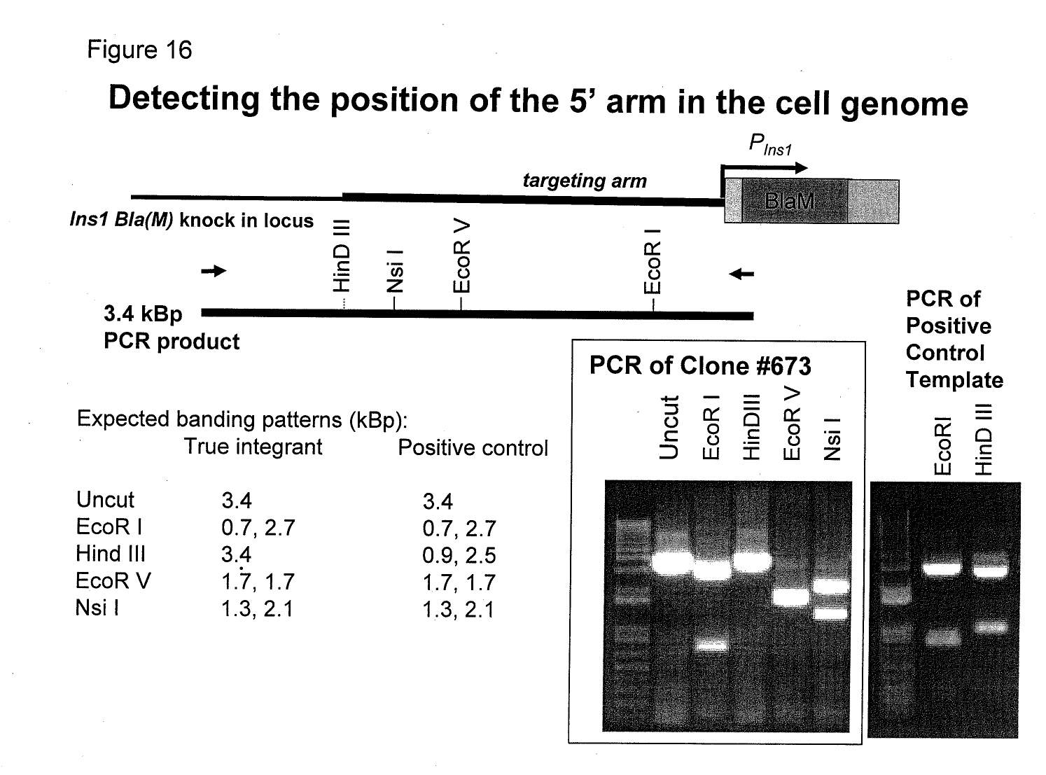

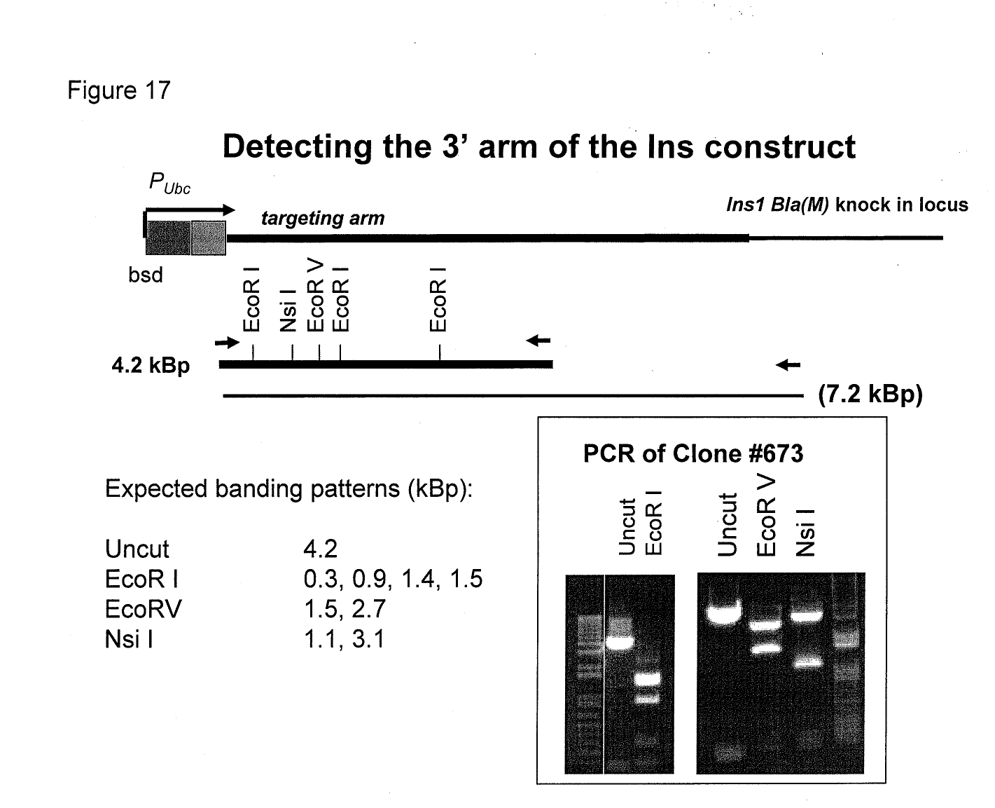

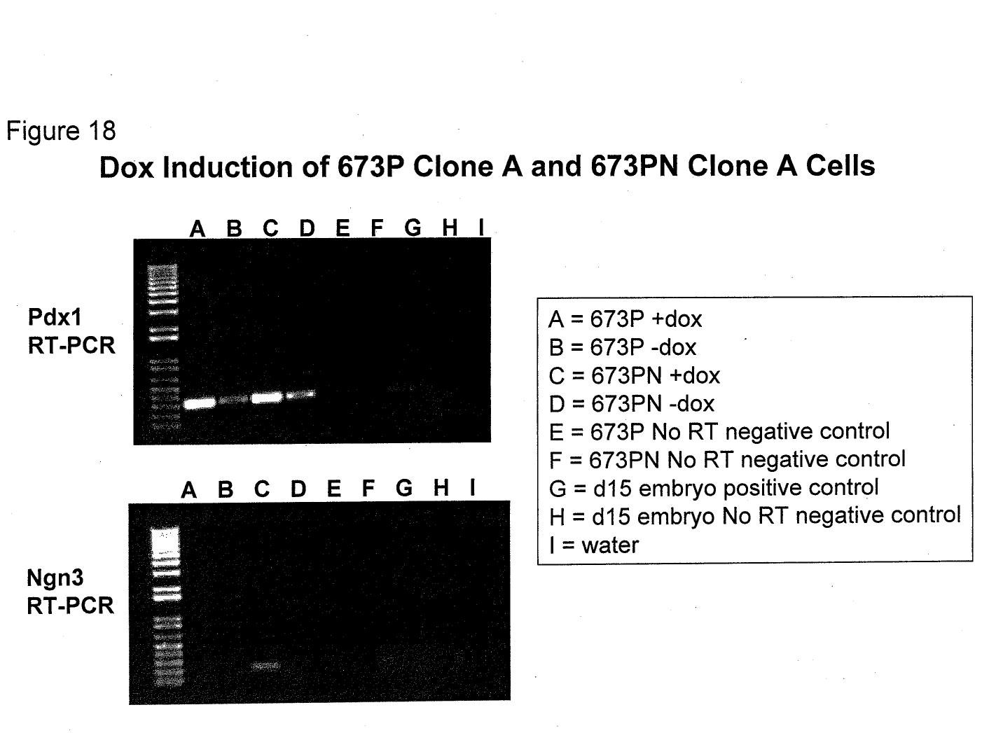

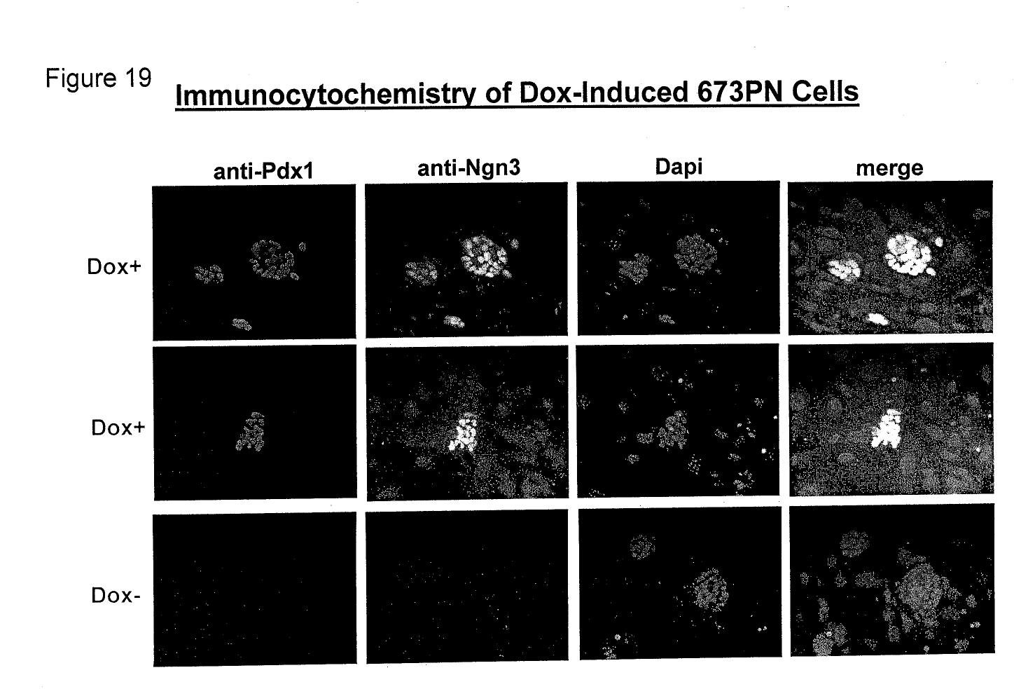

The bla gene was integrated into the genome of Ainv18 cells by homologous recombination. The target construct, Ins1-BLA3b, was electroporated into the cells followed by selection with blasticidin. Resulting clones were analyzed for BLA expression and a positive clone, designated 673 was isolated. The 673 clone, encoding the Ins1-Bla construct was then used for the introduction of Tet-pdx1 and Tet-pdx1-IRES-ngn3, via cre-lox recombination to generate cell lines 673P and 673PN, respectively. The bla and bsd genes were successfully targeted to the ins1 gene of the host cells as demonstrated by PCR (FIG. 15). PCR was used to demonstrate correct integration of the blaM gene on the 5′ (FIG. 16) and 3′ sides (FIG. 17). Dox-induced upregulation of Pdx1 in cell line 673P and Dox-induced upregulation of Pdx1 and Ngn3 in cell line 673PN cells was demonstrated by RT-PCR (FIG. 18). In addition, immunohistochemistry analysis was used to demonstrate Dox-induced expression of Pdx1 and Ngn3 in 673PN cells (FIG. 19).

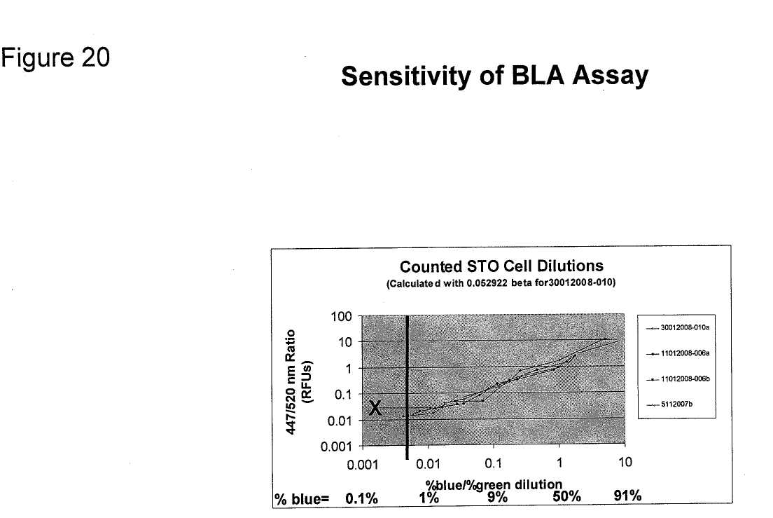

In an effort to demonstrate the sensitivity of the BLA assay, a cell line was generated in which plasmid pGeneBLAzer™ UBC (Invitrogen) was introduced into STO cells. The resulting cell line, pBLA-STO, fluoresces blue in the presence of CCF2 due to the expression of β-lactamase. The parent cell line, STO, fluoresces green in the presence of CCF2 due to the lack of β-lactamase. To demonstrate the sensitivity of the BLA assay, pBLA-STO cells mixed with wild type STO cells at various ratios. Duplicate dilution sets of three biological replicates were made and assayed with the BLA assay (Gene BLAzer™ Detection Kits, Invitrogen). Blue/green ratios were plotted against % blue/% green dilutions either based on 1) serial dilution estimates, or 2) cell counts from photos of each dilution. Based on serial dilutions, the threshold of sensitivity of the BLA assay is approximately 1% blue cells in a population of green cells. Based on cell counts, the threshold of sensitivity of the BLA assay is approximately 0.4% blue cells in a population of green cells FIG. 20 and Table 4).

TABLE 4
Sensitivity of BLA assay

| % blue | % green | % blue/% green |
|---|---|---|
| 0.00195 | 0.99805 | 0.00196 |
| 0.00391 | 0.99609 | 0.00392 |
| 0.00781 | 0.99219 | 0.00787 |
| 0.01563 | 0.98438 | 0.01587 |
| 0.03125 | 0.96875 | 0.03226 |
| 0.06250 | 0.93750 | 0.06667 |
| 0.12500 | 0.87500 | 0.14286 |
| 0.25000 | 0.75000 | 0.33000 |
| 0.50000 | 0.50000 | 1.00000 |
| 0.75000 | 0.25000 | 3.00000 |

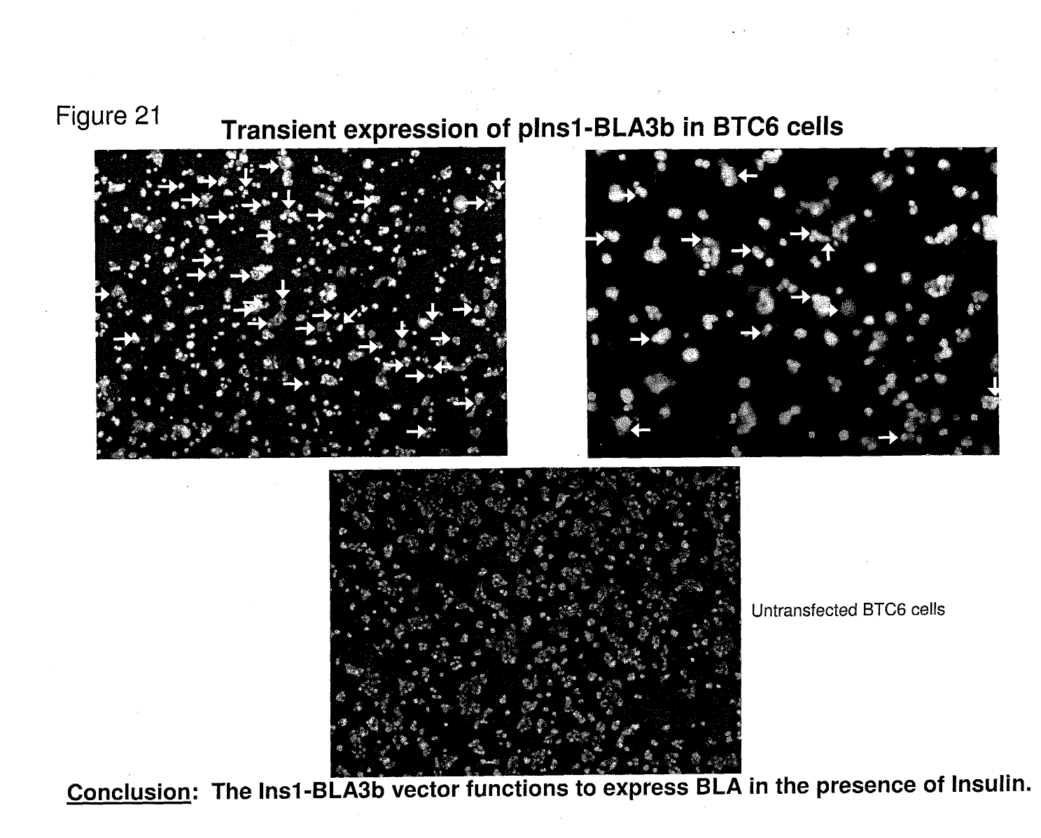

In order to test the inducibility of the Ins1-BLA expression cassette, the Ins1-BLA targeting vector was electroporated into βTC6 cells, an insulinoma cell line that expresses insulin. Cells were cultured for up to three days after electroporation and the expression of the Ins1-BLA expression cassette was determined by BLA assay. As shown in FIG. 21, the BLA reporter construct was expressed in the presence of insulin by 24 hours post-transfection.

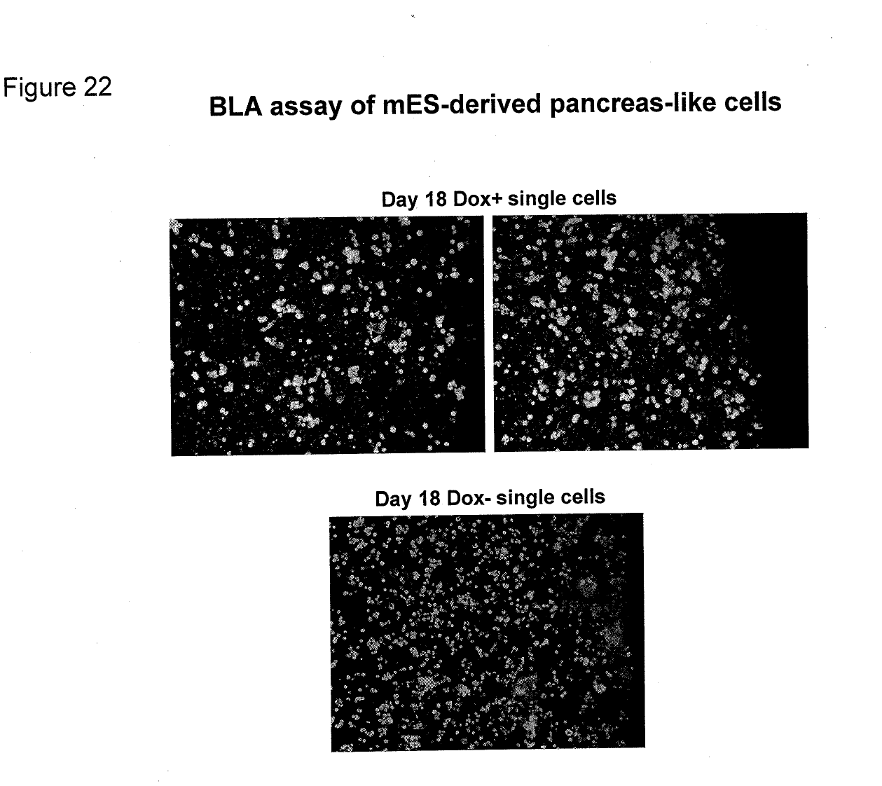

The induction the ins1 promoter during the progression of ES cells to pancreatic endocrine progenitor cells by timed overexpression of Pdx1 and Ngn3 was demonstrated using 673PN cells in which BLA expression is controlled by the Ins1 promoter and Pdx1 and Ngn3 expression is controlled by a tetracycline inducible promoter. EBs were derived from ES cells using the SFD protocol. EBs were treated with Dox starting on day 4 or maintained without Dox. At the end of the protocol, cells were dissociated, plated onto Poly-L-lysine and subjected to the BLA assay. As shown in FIG. 22, EBs that were induced to overexpress Pdx1 and Ngn3 also displayed BLA expression (blue cells) by day 18. EBs that did not overexpress Pdx1 and Ngn3 did not express BLA (green cells).

### Example 11

### Timecourse of Ins1-BLA Expression During Pancreatic Differentiation

A timecourse of Ins1-BLA expression during pancreatic differentiation is used to determine that BLA expression tracks insulin expression. 673PN cells are induced to differentiate as described in either Example 1 or Example 2. At various times after induction of Pdx1 and Ngn3 expression, cells are analyzed by RT-PCR for expression of BLA and Ins1. In addition, a sample of cells is assayed for BLA expression by a BLA assay. Results are then plotted to show tracking of BLA with insulin expression.

### Example 12

### Targeting an Insulin Reporter System to the ROSA26 Locus

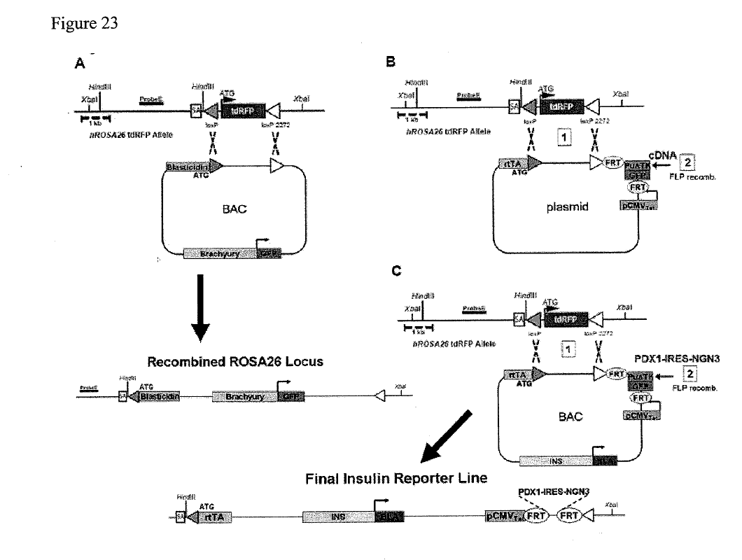

In order to generate an insulin reporter human embryonic stem cell line, the bla gene under the control of the Ins1 promoter is targeted to the ROSA26 locus in the cells. The human ROSA26 ortholog has been identified and mutated without impairing cell function (Irion, et al. 2007). Cell line Hes2.R26 tdRFP is used (ESI, Singapore; Irion et al. 2007). This cell line contains directional lox sites which may be used to test the recombinational strategy. This cell line has also been demonstrated to differentiate into all three germ layers. A bacterial artificial chromosome (BAC) containing the human brachyury locus and 160 kb of flanking DNA (CTD-2379F21) is modified using lambda-red based recombineering (Sawitzke, J. A. et al 2007 Meth. Enzymol. 421:171-199) to express GFP from the endogenous brachyury start codon (FIG. 23A). Heterologous LoxP recombination sites (LoxP and LoxP2272) are included in the BAC. A gene conferring resistance to blasticidin is located downstream of the ROSA26 splice acceptor (SA) sequence. The BAC and a Cre-recombinase expressing plasmid are electroporated into Hes2.R26 cells and recombinants are selected for resistance to blasticidin and loss of red fluorescence (tdRFP). PCR is carried out to verify correct integration in the ROSA26 locus. The resultant cell line is designated Hes2.R26T-GFP.

A tetracycline inducible system (Gossen, M. et al. 1994 Curr. Opin. Biotechnol. 5:516-520) is introduced into the ROSA26 locus (FIG. 23B). The reverse tetracycline transactivator, rtTA, is expressed from a ROSA26 promoter following an SA sequence. A destabilized GFP-IRES-PuromycinΔThymidine Kinase (PuΔTK), allowing for positive/negative selection with puromycin/ganciclovir (Chen, Y. T. and Bradley, A. 2000 Genesis 28:31-35) is included as a reporter flanked by FRT sites and is tested for inducibility. FRT site functionality is tested by replacement of GFP-IRES-PuΔTK with a cassette patterning cDNA and transient FLP recombinase expression. Clones are selected with ganciclovir followed by EB differentiation and designated Hes2.R26 TetGFP-IRES-PuΔTK.

The tetracycline system controlling Pdx1 and Ngn3 is combined with a reliable insulin reporter, Ins-BLA, at the ROSA26 locus in order to make a novel hES cell line for differentiation into pancreas-like cells and to test drugs/biologics that promote insulin expression. GFP-IRES-PuΔTK is replaced by pdx1-IRES-ngn3. The resulting cells are validated by several methods including PCR to verify targeting to the ROSA26 locus, RT-PCT and immunohistochemistry of tetracycline (or Dox) induced undifferentiated cells to demonstrate upregulation of Pdx1 and Ngn3, and reassessment of cell karyotype, cell phenotype and pluripotency. The tetracycline cassette may be separated from the BAC ends if needed for consistent expression (Kyba, M. et al. 2002 Cell 109:29-37). The resultant cell line is designated INS-BLA1 TetPDX1-NGN3.

An activin-bases pancreatic differentiation protocol is used to yield cells that co-express Bla and insulin as well as other β-islet cell markers. Growth factor additions, timing and concentrations are altered in order to optimize the number and functioning of insulin (BLA) expressing cells. Marker profiles of developing and mature human pancreas, including GCG, SST, PPY, GHRL, PTF1A, ELA1, as well as β-cell markers NEUROD1, PAX4, MAFA, NKX2, GLUT2, GCK, ABCC8, KCNJ11, PCSK1, PCSK2 (Murtaugh, 2007), are analyzed using microarrays, RT-PCR, flow cytometry, microplate reading and immunocytochemistry and are compared to Bla kinetic responses to various secretagogues. Candidate cDNAs, identified by β-islet microarray data are recombined into FRT sites to validate function and further improve pancreas characteristics and quality of insulin expressing cells.

### Example 13

### The BLA Assay Detects mIns1 Promoter Driven BLA in d22 673PN-Derived Pancreas-Like Cells

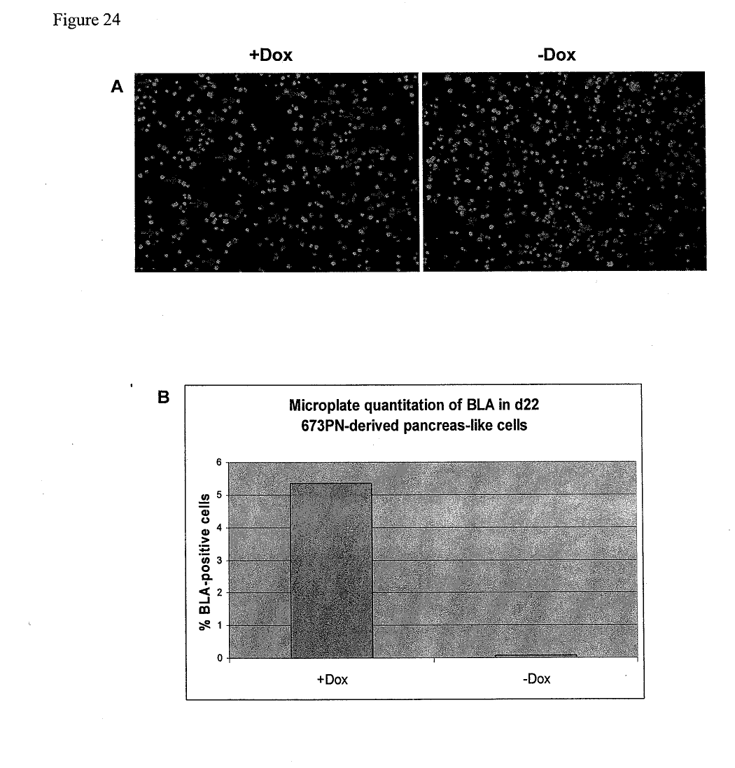

673PN cells were differentiated for 22 days using the SFD protocol as described for Example 2. Expression of Pdx1 and Ngn3 was induced by Dox between days 4-22. The cells were then dissociated into single cells, plated on Poly-L-lysine, and assayed with the BLA assay. Fluorescent microscopy revealed blue, BLA-positive cells in Dox-induced samples, indicating mIns1 promoter activity (FIG. 24A). Approximately 6% of the Dox-induced cells were blue, as determined by cell counts of blue and green cells in random photographs. No blue cells were evident in −Dox samples. BLA was quantitated in the same d22 cells with a microplate reader (FIG. 24B). Calculations of the background-corrected blue/green ratio indicated that 5.3% of the cells expressed BLA, which correlates well with the fluorescent microscopy cell counts. This cell line will serve as a powerful tool, for example, in the optimization of ES-derived pancreatic differentiation and as a high throughput screen for identifying small molecules and/or biologics that either upregulate the expression of insulin or increase the production of beta islet cells, thus improving the efficiency of identification of drug candidates for the treatment of diabetes.

### Example 14

### Ins1 and BLA are Induced in 673PN Cells in Response to Introduction of MafA

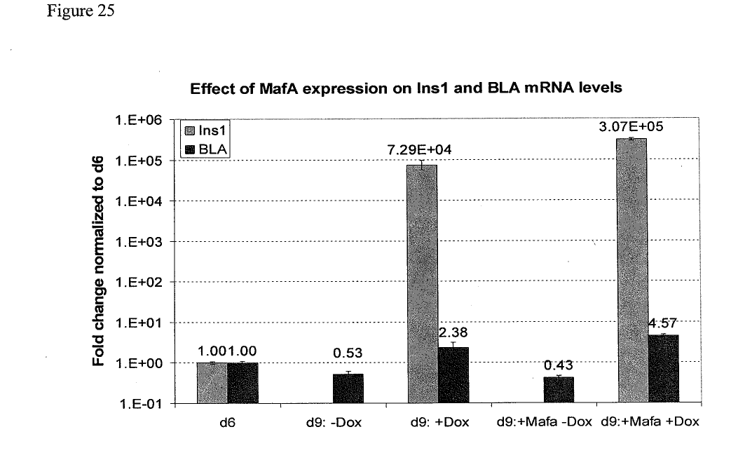

673PN cells were differentiated for 9 days using the SP protocol as described in Example 1. A vector encoding MafA under the control of the CMV promoter (vector derived from pCMV-Sport6, Invitrogen) or an empty vector was introduced to the cells at day 6 by electroporation. Pdx1 and Ngn3 were induced in half the samples with Dox between days 6-9. Ins1 and BLA gene expression was measured on day 9 by quantitative RT-PCR (FIG. 25). Introduction of MafA induces Ins1 expression over the baseline pancreatic differentiation protocol. Importantly, expression of BLA also demonstrates a concomitant induction indicating tracking of Ins1 expression with BLA.

### Example 15

### Pancreatic Endocrine Progenitors from iPS Cells

Pancreatic endocrine progenitor cells are derived from iPS cells by differentiation of iPS cells into endoderm by treatment with activin followed by expression of Pdx1 and Ngn3 and in some samples, MafA, in the endoderm cells. In some samples, polynucleotides expressing Pdx1, Ngn3 and MafA are stably introduced to iPS cells prior to differentiation. In some samples, polynucleotides expressing Pdx1, Ngn3 and MafA are introduced to endoderm cells derived from iPS cells. In some samples, polynucleotides expressing Pdx1, Ngn3 and MafA are under the control of an inducible promoter. To differentiate iPS cells to pancreatic endocrine progenitor cells, a population of undifferentiated iPS cells maintained on MEF feeder cells is used. On about day −4, cells are plated on gelatinized culture dishes in the absence of MEF feeder cells. On about day −2 cells are passaged in a pre-differentiation step. On day 0, EBs are induced by culture in SFD complete medium. On about day 2, EBs are dissociated and replated in the presence of activin A. On about day 4, EBs are reaggregated and Pdx1, Ngn3 and MafA expression is induced; for example, by addition of Dox to the media. On about day 6, cells are expanded on low attachment plates. Induction of expression of Pdx1, Ngn3 and MafA is continued. On about days 9, 11 and 13 cells are fed and induction of expression of Pdx1, Ngn3 and MafA is continued. On about day 16, cells are harvested and analyzed. Cells are analyzed for pancreatic endocrine progenitor cell characteristics by a number of methods known in the art including, but not limited to RT-PCR, immunohistochemistry and enzyme assays. In some samples, a polynucleotide encoding a reporter gene such as beta-lactamase or GFP under the control of insulin-1 regulatory elements is also stably introduced into to the iPS cells. In these samples, cells can be assayed for development of pancreatic endocrine progenitor characteristics by BLA assay or FACS.

### Example 16

### Induction of Pancreatic Endocrine Progenitors from iPSC

Another example of a method to generate pancreatic endocrine progenitor cell from iPS cells in which Pdx1, Ngn3 and in some samples MafA are stably introduced is provided as follows. Undifferentiated iPS cells are maintained on MEF feeder cells. On about day −4, cells are plated on gelatinized culture dishes in the absence of MEF feeder cells. On about day −2 cells are passaged in a pre-differentiation step. On day 0, iPS cells are plated as a monolayer in SFD complete medium. On about day 2, cells are dissociated and replated in the presence of activin A. On about day 4, cells are dissociated and Pdx1, Ngn3 and MafA expression is induced; for example, by addition of Dox to the media. On about day 6, cells are expanded. Induction of expression of Pdx1, Ngn3 and MafA is continued. On about days 9, 11 and 13 cells are fed and induction of expression of Pdx1, Ngn3 and MafA is continued. In some samples, cells are harvested and analyzed on about day 16. Cells are analyzed for pancreatic endocrine progenitor cell characteristics by a number of methods known in the art including, but not limited to RT-PCR, immunohistochemistry and enzyme assays. In some samples, a polynucleotide encoding a reporter gene, such as beta-lactamase or GFP, under the control of insulin-1 regulatory elements is also stably introduced into to the iPS cells. In these cases, cells are assayed for development of pancreatic endocrine progenitor characteristics by BLA assay or FACS. The resulting pancreatic endocrine progenitor cells are maintained as a monolayer.

All publications, patents, patent applications, internet sites, and accession numbers/database sequences (including both polynucleotide and polypeptide sequences) cited herein are hereby incorporated by reference herein in their entirety for all purposes to the same extent as if each individual publication, patent, patent application, internet site, or accession number/database sequence were specifically and individually indicated to be so incorporated by reference.

We claim:

1. A pluripotent stem cell modified to overexpress Pdx1 and Ngn3.

2. A pluripotent stem cell of claim 1, wherein expression of Pdx1 and Ngn3 are under the control of one or more inducible promoters.

3. The pluripotent stem cell of claim 1, wherein the cell is an embryonic stem cell or an induced pluripotent stem (iPS) cell.

4. The cell of claim 1, wherein the overexpression of Pdx1 and Ngn3 is simultaneous.

5. The cell of claim 1, wherein the overexpression of Pdx1 and Ngn3 is sequential.

6. The cell of claim 1 further comprising a reporter molecule.

7. The cell of claim 6, wherein the reporter molecule is operably linked to a promoter expressed in pancreatic endocrine progenitor cells or derivatives thereof but not expressed in primitive endoderm.

8. The cell of claim 2 further comprising a reporter molecule.

9. The cell of claim 8, wherein the reporter molecule is operably linked to a promoter expressed in pancreatic endocrine progenitor cells or derivatives thereof but not expressed in primitive endoderm.

10. The cell of claim 1 further modified to overexpress MafA.

11. The cell of claim 2 further modified to overexpress MafA under the control of an inducible promoter.

12. The cell of claim 11 further comprising a reporter molecule.

13. The cell of claim 12, wherein the reporter molecule is operably linked to a promoter expressed in pancreatic endocrine progenitor cells or derivatives thereof but not expressed in primitive endoderm.

14. A method of producing a pluripotent stem cell to overexpress Pdx1 and Ngn3, the method comprising the step of introducing nucleic acid encoding Pdx1 and Ngn3 into the cell.

15. The method of claim 14, wherein the pluripotent stem cell is an embryonic stem cell or an iPS cell.

16. The method of claim 14, wherein the nucleic acid encoding Pdx1 and the nucleic acid encoding Ngn3 are operably linked to one or more inducible promoters.

17. The method of claim 14, wherein the method further comprises the step of introducing a reporter molecule to the cell.

18. The method of claim 17, wherein the reporter molecule is operably linked to a promoter expressed in pancreatic endocrine progenitor cells or derivatives thereof but not expressed in primitive endoderm.

19. A method of producing a pluripotent stem cell to overexpress Pdx1, Ngn3 and MafA; the method comprising the steps of:
a) introducing nucleic acid encoding Pdx1 and Ngn3 into the cells, and
b) introducing nucleic acid encoding MafA into the cells.

20. The method of claim 19, wherein the pluripotent stem cell is an embryonic stem cell or an iPS cell.

21. The method of claim 19, wherein the nucleic acid encoding Pdx1 and the nucleic acid encoding Ngn3 are operably linked to one or more inducible promoters.

22. The method of claim 19, wherein the nucleic acid encoding MafA is operably linked to an inducible promoter.

23. The method of claim 19, wherein the method further comprises the step of introducing a reporter molecule to the cell.

24. The method of claim 23, wherein the reporter molecule is operably linked to a promoter expressed in pancreatic endocrine progenitor cells or derivatives thereof but not expressed in primitive endoderm.

25. A method of producing pancreatic endocrine progenitor cells from pluripotent stem cells, the method comprising the steps of
a) producing definitive endoderm cells from the pluripotent stem cells,
b) expressing Pdx1 and Ngn3 in the definitive endoderm cells, and
c) culturing the cells for sufficient time to identify pancreatic endocrine progenitor cells.

26. The method of claim 25, wherein the pluripotent stem cells are embryonic stem cells or iPS cells.

27. The method of claim 25, wherein the pancreatic endocrine progenitor cells are identified by expression of insulin.

28. The method of claim 25, wherein the method includes an additional step of culturing the pancreatic endocrine progenitor cells in a monolayer.

29. A method of producing pancreatic endocrine progenitor cells from pluripotent stem cells, the method comprising the steps of
a) producing definitive endoderm cells from the pluripotent stem cells,
b) initiating expression of Pdx1 in the definitive endoderm cells,
c) analyzing the Pdx1-expressing cells for the expression of insulin mRNA,
d) initiating expression of Ngn3 in the Pdx1-expressing cells, and
e) culturing the Pdx1/Ngn3-expressing cells for sufficient time to identify pancreatic endocrine progenitor cells.

30. The method of claim 29, wherein the pluripotent stem cells are embryonic stem cells or iPS cells.

31. The method of claim 29, wherein the pancreatic endocrine progenitor cells are identified by expression of insulin.

32. The method of claim 29, wherein the method includes an additional step of culturing the pancreatic endocrine progenitor cells in a monolayer.

33. A method of producing primitive beta-islet cells from pluripotent stem cells, the method comprising the steps of
a) producing definitive endoderm cells from the pluripotent stem cells,
b) expressing Pdx1 and Ngn3 in the definitive endoderm cells,
c) culturing the Pdx1/Ngn3-expressing cells for sufficient time to identify pancreatic endocrine progenitor cells by measuring expression of insulin,
d) expressing MafA in the pancreatic endocrine progenitor cells, and
e) culturing the cells for sufficient time to identify primitive beta-islet cells by measuring secretion of insulin.

34. The method of claim 33, wherein the pluripotent stem cells are embryonic stem cells or iPS cells.

35. The method of claim 33, wherein the method includes an additional step of culturing the pancreatic endocrine progenitor cells in a monolayer.

36. A method of producing pancreatic endocrine progenitor cells from pluripotent stem cells, the method comprising the steps of:
a) preparing embryonic bodies (EB) from the pluripotent stem cells of claim 2,
b) dissociating the cells and incubating the cells in the presence of activin A on about day 2,
c) dissociating the cells and inducing expression of Pdx1 and Ngn3 starting about day 4-about day 6,
d) plating the cells on low attachment plates about day 6-about day 9, and
e) culturing the cells for sufficient time to identify pancreatic endocrine progenitor cells.

37. A method of producing pancreatic endocrine progenitor cells from pluripotent stem cells, the method comprising the steps of:
a) culturing pluripotent stem cells of claim 2 as a monolayer,
b) dissociating the cells and incubating the cells in the presence of activin A on about day 2,
c) dissociating the cells and inducing expression of Pdx1 and Ngn3 starting about day 4-about day 6,
d) plating the cells on about day 6-about day 9, and
e) culturing the cells for sufficient time to identify pancreatic endocrine progenitor cells.

38. The method of claim 36 or 37, wherein the pluripotent stem cells are embryonic stem cells or iPS cells.

39. The method of claim 36 or 37, wherein the pancreatic endocrine progenitor cells are identified by expression of insulin.

40. The method of claim 36 or 37 wherein a nucleic acid encoding a reporter molecule is introduced to the cells prior to identifying pancreatic endocrine progenitor cells.

41. The method of claim 40, wherein the nucleic acid encoding a reporter molecule is operably linked to a promoter expressed in pancreatic endocrine progenitor cells or derivatives thereof but not expressed in primitive endoderm.

42. A method of producing pancreatic endocrine progenitor cells from pluripotent stem cells, the method comprising the steps of:
a) preparing embryonic bodies (EB) from the pluripotent stem cell of claim 9,
b) dissociating the cells and incubating the cells in the presence of activin A on about day 2,
c) dissociating the cells and inducing expression of Pdx1 and Ngn3 starting about day 4-about day 6,
d) plating the cells on low attachment plates about day 6-about day 9, and
e) culturing the cells for sufficient time to identify pancreatic endocrine progenitor cells by identifying cells expressing the reporter molecule.

43. A method of producing pancreatic endocrine progenitor cells from pluripotent stem cells, the method comprising the steps of:
a) incubating a population of cells of claim 9 to initiate differentiation,
b) dissociating the cells and incubating the cells in the presence of activin A on about day 2,
c) dissociating the cells and inducing expression of Pdx1 and Ngn3 starting about day 4-about day 6,
d) plating the cells on about day 6-about day 9,
e) culturing the cells for sufficient time to identify pancreatic endocrine progenitor cells by identifying cells expressing the reporter molecule.

44. The method of claim 42 or 43, wherein the pluripotent stem cells are embryonic stem cells or iPS cells.

45. A method of producing primitive beta-islet cells from pluripotent stem cells, the method comprising the steps of:
a) preparing embryonic bodies (EB) from the pluripotent stem cell of claim 11,
b) dissociating the cells and incubating the cells in the presence of activin A on about day 2,
c) dissociating the cells and inducing expression of Pdx1 and Ngn3 starting about day 4-about day 6,
d) inducing expression of MafA,
e) plating the cells on low attachment plates about day 6-about day 9, and
f) culturing the cells for sufficient time to identify primitive beta-islet cells.

46. A method of producing primitive beta-islet cells from pluripotent stem cells, the method comprising the steps of:
a) incubating a population of cells of claim 11 to initiate differentiation,
b) dissociating the cells and incubating the cells in the presence of activin A on about day 2,
c) dissociating the cells and inducing expression of Pdx1 and Ngn3 starting about day 4-about day 6,
d) inducing expression of MafA,
e) plating the cells on about day 6-about day 9, and
f) culturing the cells for sufficient time to identify pancreatic endocrine progenitor cells.

47. The method of claim 45 or 46, wherein the pluripotent stem cells are embryonic stem cells or iPS cells.

48. A method of producing primitive beta-islet cells from pluripotent stem cells, the method comprising the steps of:
a) preparing embryonic bodies (EB) from the pluripotent stem cell of claim 13,
b) dissociating the cells and incubating the cells in the presence of activin A on about day 2,
c) dissociating the cells and inducing expression of Pdx1 and Ngn3 starting about day 4-about day 6,
d) inducing expression of MafA,
e) plating the cells on low attachment plates about day 6-about day 9, and
f) culturing the cells for sufficient time to identify primitive beta-islet cells by identifying cells expressing the reporter molecule.

49. A method of producing primitive beta-islet cells from pluripotent stem cells, the method comprising the steps of:
a) incubating a population of cells of claim 13 to initiate differentiation,
b) dissociating the cells and incubating the cells in the presence of activin A on about day 2,
c) dissociating the cells and inducing expression of Pdx1 and Ngn3 starting about day 4-about day 6,
d) inducing expression of MafA,
e) plating the cells on about day 6-about day 9, and
f) culturing the cells for sufficient time to identify pancreatic endocrine progenitor cells by identifying cells expressing the reporter molecule.

50. The method of claim 48 or 49, wherein the pluripotent stem cells are embryonic stem cells or iPS cells.

51. A method of producing pancreatic endocrine progenitor cells from pluripotent stem cells, the method comprising the steps of:
a) culturing a population of cells of claim 2 to initiate differentiation on about day −4,
b) passaging the cells on about day −2,
c) preparing EBs from the pluripotent cells on about day 0,
d) dissociating the cells and incubating the cells in the presence of activin A on about day 2,
e) dissociating the cells, inducing expression of Pdx1 and Ngn3 starting about day 4-about day 6
f) plating the cells on about day 6-about day 9,
g) culturing the cells for sufficient time to identify pancreatic endocrine progenitor cells.

52. A method of producing pancreatic endocrine progenitor cells from embryonic stem cells, the method comprising the steps of:
a) culturing a population of cells of claim 2 to initiate differentiation on about day −4,
b) passaging the cells on about day −2,
c) passaging the cells maintained as monolayer on about day 0,
d) dissociating the cells and incubating the cells in the presence of activin A on about day 2,
e) dissociating the cells, inducing expression of Pdx1 and Ngn3 starting about day 4-about day 6
f) plating the cells on about day 6-about day 9,
g) culturing the cells for sufficient time to identify pancreatic endocrine progenitor cells.

53. The method of claim 51 or 52, wherein the pluripotent stem cells are embryonic stem cells or iPS cells.

54. A method of producing pancreatic endocrine progenitor cells from pluripotent stem cells, the method comprising the steps of:
a) culturing a population of cells of claim 9 to initiate differentiation on about day −4,
b) passaging the cells on about day −2,
c) preparing EBs from the pluripotent stem cells on about day 0,
d) dissociating the cells and incubating the cells in the presence of activin A on about day 2,
e) dissociating the cells, inducing expression of Pdx1 and Ngn3 in the cells starting about day 4-about day 6
f) plating the cells on about day 6-about day 9,
g) culturing the cells for sufficient time to identify pancreatic endocrine progenitor cells by identifying cells expressing the reporter molecule.

55. A method of producing pancreatic endocrine progenitor cells from pluripotent stem cells, the method comprising the steps of:
a) culturing a population of cells of claim 9 to initiate differentiation on about day −4,
b) passaging the cells on about day −2,
c) passaging the cells maintained as monolayer on about day 0,
d) dissociating the cells and incubating the cells in the presence of activin A on about day 2,
e) dissociating the cells, inducing expression of Pdx1 and Ngn3 in the cells starting about day 4-about day 6
f) plating the cells on about day 6-about day 9,
g) culturing the cells for sufficient time to identify pancreatic endocrine progenitor cells by identifying cells expressing the reporter molecule.

56. The method of claim 54 or 55, wherein the pluripotent stem cells are embryonic stem cells or iPS cells.

57. A method of producing primitive beta-islet cells from embryonic stem cells, the method comprising the steps of:
a) culturing a population of cells of claim 11 to initiate differentiation on about day −4,
b) passaging the cells on about day −2,
c) preparing EBs from pluripotent stem cells on about day 0,
d) dissociating the cells and incubating the cells in the presence of activin A on about day 2,
e) dissociating the cells and inducing expression of Pdx1, Ngn3 and MafA in the cells starting about day 4-about day 6,
f) plating the cells on about day 6-about day 9,
g) culturing the cells for sufficient time to identify pancreatic endocrine progenitor cells.

58. A method of producing primitive beta-islet cells from pluripotent stem cells, the method comprising the steps of:
a) culturing a population of cells of claim 11 to initiate differentiation on about day −4,
b) passaging the cells on about day −2,
c) passaging the cells maintained as monolayer on about day 0,
d) dissociating the cells and incubating the cells in the presence of activin A on about day 2,
e) dissociating the cells, inducing expression of Pdx1, Ngn3 and MafA in the cells starting about day 4-about day 6
f) plating the cells on about day 6-about day 9,
g) culturing the cells for sufficient time to identify pancreatic endocrine progenitor cells.

59. The method of claim 57 or 58 wherein the pluripotent stem cells are embryonic stem cells or iPS cells.

60. A method of producing primitive beta-islet cells from embryonic stem cells, the method comprising the steps of:
a) culturing a population of cells of claim 13 to initiate differentiation on about day −4,
b) passaging the cells on about day −2,
c) preparing EBs from pluripotent stem cells on about day 0,
d) dissociating the cells and incubating the cells in the presence of activin A on about day 2,
e) dissociating the cells and inducing expression of Pdx1, Ngn3 and MafA in the cells starting about day 4-about day 6,
f) plating the cells on about day 6-about day 9,
g) culturing the cells for sufficient time to identify pancreatic endocrine progenitor cells by identifying cells expressing the reporter molecule.

61. A method of producing primitive beta-islet cells from pluripotent stem cells, the method comprising the steps of:
a) culturing a population of cells of claim 11 to initiate differentiation on about day −4,
b) passaging the cells on about day −2,
c) passaging the cells maintained as monolayer on about day 0,
d) dissociating the cells and incubating the cells in the presence of activin A on about day 2,
e) dissociating the cells, inducing expression of Pdx1, Ngn3 and MafA in the cells starting about day 4-about day 6
f) plating the cells on about day 6-about day 9,
g) culturing the cells for sufficient time to identify pancreatic endocrine progenitor cells by identifying cells expressing the reporter molecule.

62. The method of claim 60 or 61 wherein the pluripotent stem cells are embryonic stem cells or iPS cells.

63. A method of screening a compound for its ability to modulate pancreatic endocrine cell function, comprising combining the compound with an pancreatic endocrine progenitor cell according to claim 25, determining any phenotypic or metabolic changes in the cell that result from being combined with the compound, and correlating the change with an ability of the compound to modulate secretion of insulin, glucagon, gherlin, or somatostatin or proliferation of insulin secreting cells.

64. A method of screening a compound for its ability to modulate beta-islet cell function, comprising combining the compound with an pancreatic endocrine progenitor cell according to claim 33, determining any phenotypic or metabolic changes in the cell that result from being combined with the compound, and correlating the change with an ability of the compound to modulate secretion of insulin or proliferation of insulin secreting cells.

65. A method of screening a compound for its ability to modulate pancreatic endocrine cell function, comprising combining the compound with a pancreatic endocrine progenitor cell according to claim 25, culturing the cells for varying amounts of time, determining any phenotypic or metabolic changes in the cell that result from being combined with the compound, and correlating the phenotypic or metabolic change with the time of culturing the cells.

66. A method of screening a compound for its ability to modulate pancreatic endocrine cell function, comprising isolating pancreatic endocrine progenitor cells that express Pdx1 and Ngn3 according to claim 25 at fixed time points following induction of differentiation, combining the compound and the isolated cells, and determining any phenotypic or metabolic changes in the cell that result from being combined with the compound.

67. A method of screening a compound for its ability to modulate pancreatic endocrine cell function, comprising combining the compound with an pancreatic endocrine progenitor cell according to claim 25, determining any phenotypic or metabolic changes in the cell that result from being combined with the compound, and correlating the change with an ability of the compound to modulate secretion of insulin.

68. A method of screening a compound for its ability to modulate primitive beta-islet cell function, comprising combining the compound with a primitive beta-islet cell according to claim 33, determining any phenotypic or metabolic changes in the cell that result from being combined with the compound, and correlating the change with an ability of the compound to modulate secretion of insulin.

69. A method of screening a compound for its ability to modulate pancreatic endocrine cell function, comprising combining the compound with a pancreatic endocrine progenitor cell according to claim 25; wherein the pancreatic endocrine progenitor cell further comprises a reporter molecule operably linked to a promoter expressed in pancreatic endocrine progenitor cells or derivatives thereof but not expressed in primitive endoderm; and determining changes in expression of the reporter molecule.

70. A method of pancreatic cell therapy comprising administering to a subject in need of such treatment a composition comprising pancreatic endocrine progenitor cells produced by the method of claim 25.

71. A method of pancreatic cell therapy comprising administering to a subject in need of such treatment a composition comprising primitive beta-islet cells produced by the method of claim 33.

72. A method of pancreatic cell therapy comprising administering to a subject in need of such treatment a composition comprising pancreatic endocrine progenitor cells produced by the method of claim 25; wherein the cells are autologous to the subject.

73. A method of pancreatic cell therapy comprising administering to a subject in need of such treatment a composition comprising primitive beta-islet cells produced by the method of claim 33; wherein the cells are autologous to the subject.

74. A method of pancreatic cell therapy comprising administering to a subject in need of such treatment a composition comprising pancreatic endocrine progenitor cells produced by the method of claim 25; wherein the cells are allogeneic to the subject.

75. A method of pancreatic cell therapy comprising administering to a subject in need of such treatment a composition comprising primitive beta-islet cells produced by the method of claim 33; wherein the cells are allogeneic to the subject.

76. A composition comprising pancreatic endocrine progenitor cells produced by the method of claim 25.

77. A composition comprising primitive beta-islet cells produced by the method of claim 33.

78. Use of pancreatic endocrine progenitor cells produced by the method of claim 25 in the manufacture of a medicament for treatment of an individual in need of pancreatic cell therapy.

79. Use of pancreatic endocrine progenitor cells produced by the method of claim 25 in the manufacture of a medicament for the treatment of a condition associated with deficiency of a pancreatic endocrine hormone.

80. The use of claim 79, wherein the pancreatic endocrine hormone is selected from the group consisting of insulin, glucagon, somatostatin, gherlin and pancreatic polypeptide.

81. The use of claim 80, wherein the pancreatic endocrine hormone is insulin.

82. The use of claim 81, wherein the condition associated with deficiency of a pancreatic endocrine hormone is diabetes.

83. Use of primitive beta-islet cells produced by the method of claim 33 in the manufacture of a medicament for treatment of an individual in need of pancreatic cell therapy.

84. Use of primitive beta-islet cells produced by the method of claim 33 in the manufacture of a medicament for the treatment of a condition associated with a deficiency of beta-islet cell function.

85. The use of claim 84, wherein the condition is diabetes.
